SEQUENCE LISTING (154 sequences)

SEQ ID NO: 1 - protein, 117 aa, Mus musculus
  source 1..117: note = G2D11 VH domain
QVQMQQSDAELVKPGASVKISCKASGYIFADHAIHWVKRKPEQGLEWIGYISPGNDDIKYNEKFKGKATLTADKSSSTAYMQLNSLTSEDSAVYFCKRSLPGTFDYWGQGTTLTVSS

SEQ ID NO: 2 - protein, 108 aa, Mus musculus
  source 1..108: note = G2D11 VL domain
DVVMTQSHKFMSTSVGDRVSITCKASQDVGTAVAWYQQKPGQSPKLLIYWASTRHTGVPDRFTGSGSGTDFTLTISNVQSEDLADYFCQQYSSYPLTFGGGTKLEMKR

SEQ ID NO: 3 - protein, 111 aa, Mus musculus
  source 1..111: note = 5E5 VH domain
QSDAELVKPGSSVKISCKASGYTFTDHAIHWVKQKPEQGLEWIGHFSPGNTDIKYNDKFKGKATLTVDRSSSTAYMQLNSLTSEDSAVYFCKTSTFFFDYWGQGTTLTVSS

SEQ ID NO: 4 - protein, 112 aa, Mus musculus
  source 1..112: note = 5E5 VL domain
ELVMTQSPSSLTVTAGEKVTMICKSSQSLLNSGDQKNYLTWYQQKPGQPPKLLIFWASTRESGVPDRFTGSGSGDFTLTISSVQAEDLAVYYCQNDYSYPLTFGAGTKLELK

SEQ ID NO: 5 - protein, 115 aa, Mus musculus
  source 1..115: note = 5F7 VH domain
QVQLQQSDAELVKPGASVKISCKASGYTFTDHAIHWVKQRPEQGLEWIGYFSPGNDDIHYNEKFEGKATLTADKSSSTAYMQLNSLTSEDSAVYFCKRSYDKDCWGQGTTLTVSS

SEQ ID NO: 6 - protein, 126 aa, Mus musculus
  source 1..126: note = 5F7 VL domain
KDIVMSQSPSSLGVSVGEKVTMSCKSSQSLLYSTNQKNYQSLLYSTNQKNYLAWYQQKPGQSPKLLIYWVSNRKSGVPDRFTGSGSGTDFTLTISSVKAEDLAVYYCQQYYRYPLTFGAGTKLELK

SEQ ID NO: 7 - protein, 119 aa, Mus musculus
EQVQLQQSDAELVKPGASVKISCKASGYTFTDHAIHWVKQKPEQGLEWIGYFSPGNGDFKYNEKFKGKATLTADKSSSTAFMQLNRLTSEDSAVYFCKTSLPGNHFDYWGQGTTLTVSS

SEQ ID NO: 8 - protein, 116 aa, Mus musculus
  source 1..116: note = 2D9 VL domain
DIVMSQSPSSLAVSVGEKVTMSCKSSQSLLYSSDQKNYLAWYQQKPGQSPKLLIYWASTRESGVPDRFTGSGSGTDFTLTISSVKAEDLAVYYCQQCYSYPFTFGSGTKLERKRGG

SEQ ID NO: 9 - protein, 117 aa, Mus musculus
  source 1..117: note = D3-TnMUC1 VL domain
DYKDIQMTQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYPLTFGAGTKLEMKR

SEQ ID NO: 10 - protein, 111 aa, Mus musculus
  source 1..111: note = A3-TnMUC1 VL domain
DYKDIVMTQSQKFMSTSVGDRVSITCKASQNVGTAVAWYQQKPGQSPKLLIYSASNRYTGVPDRFTGSGSGTDFTLTISNVQSEDLADYFCLQHWNYPLTFGGGTKLEIKR

SEQ ID NO: 11 - protein, 111 aa, Mus musculus
  source 1..111: note = D2-TnMUC1 VL domain
DYKDIQMTQSHKFMSTSVGDRVSITCKASQDVGTAVAWYQQKPGQSPKLLIYWASTRHTGVPDRFTGSGSGTDFTLTISNVQSEDLADYFCLQHWNYPLTFGGGTKLEIKR

SEQ ID NO: 12 - protein, 111 aa, Mus musculus
  source 1..111: note = Ori-TnCD43 VL domain
DYKDIQMTQSPASLSASVGETVTITCRASENIYSYLAWYQQKQGKSPQLLVYNAKTLAEGVPSRFSGSGSGTQFSLKINSLQPEDFGSYYCQHHYGTPYTFGGGTKLEIKR

SEQ ID NO: 13 - protein, 117 aa, Mus musculus
DYKDIVMTQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYPWTFGGGTKLEIKR

SEQ ID NO: 14 - protein, 111 aa, Mus musculus
  source 1..111: note = A1-TnCD43 VL domain
DYKDIVMTQSPASLSASVGETVTITCRASENIYSYLAWYQQKQGKSPQLLVYNAKTLAEGVPSRFSGSGSGTQFSLKINSLQSEDFGSYYCQHHYGTPYTFGGGTKLEIKR

SEQ ID NO: 15 - protein, 111 aa, Mus musculus
  source 1..111: note = F4-TnCD43 VL domain
DYKDIQMTQSPASLSASVGETVTITCRASENIYSYLAWYQQKQGKSPQLLVYNAKTLAEGVPSRFSGSGSGTQYSLKINSLQPEDFGSYYCQHFWSTPYTFGGGTKLEMKR

SEQ ID NO: 16 - protein, 111 aa, Mus musculus
  source 1..111: note = C5-TnCD43 VL domain
DYKDVQMTQSHKFMSTSVGDRVSITCKASQDVSTAVAWYQQKPGQSPKLLIYWASTRHTGVPDRFTGSGSGTDYTLTISSVQAEDLALYYCQQHYSTPYTFGGGTKLEIKR

SEQ ID NO: 17 - protein, 111 aa, Mus musculus
  source 1..111: note = A7-TnCD43 VL domain
DYKDIVMTQSHKFMSTSVGDRVSITCKASQDVGTAVAWYQQKPGQSPKLLIYWASTRHTGVPDRFTGSGSGTDFTLTISNVQSEDLADYFCQQYSSYPYTFGGGTKLEMKR

SEQ ID NO: 18 - protein, 117 aa, Mus musculus
  source 1..117: note = D3-TnCD43 VL domain
DYKDIVMTQSPSSLAVSAGEKVTMSCKSSQSLLNSRTRKNYLAWYQQKPGQSPKLLIYWASTRESGVPDRFTGSGSGTDFTLTISNVQSEDLAEYFCQQYNSYPLTFGAGTKLEIKR

SEQ ID NO: 19 - protein, 111 aa, Mus musculus
  source 1..111: note = G3-TnCD43 VL domain
DYKDVVMTQSQKFMSTSVRDRVSITCKASQNVGTAVAWYQQKPGQSPKLLIYSASYRYSGVPDHFTGSGSGTDFTLTISNVQSEDLAEYFCQQYYSYPYTFGGGTKLEIKR

SEQ ID NO: 20 - protein, 117 aa, Mus musculus
  source 1..117: note = D7-TnCD43 VL domain
DYKDLVLTQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYPWTFGGGTKLEMKR

SEQ ID NO: 21 - protein, 117 aa, Mus musculus
  source 1..117: note = H2-TnCD43 VL domain
DYKDIVMTQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYPYTFGGGTKLEIKR

SEQ ID NO: 22 - protein, 113 aa, Mus musculus
  source 1..113: note = C4-STnMUC1 VL domain
IVMTQSPSSLAVSAGEKVTMSCKSSQSLLNSRTRKNYLAWYQQKPGQSPKLLIYWASTRHTGVPDRFTGSGSGTDFTLTISNVQSEDLAEYFCQQYNSYPYTFGGGTKLEIKR

SEQ ID NO: 23 - protein, 113 aa, Mus musculus; identical to SEQ ID NO: 22
  source 1..113: note = D3-STnMUC1 VL domain

SEQ ID NO: 24 - protein, 112 aa, Mus musculus
  source 1..112: note = C7-STnMUC1 VL domain
VVVTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPRTFGGGTKLEIKR

SEQ ID NO: 25 - protein, 116 aa, Mus musculus
  source 1..116: note = 3F1 VH domain
QVQLKQSDAELVKPGASVKISCKASGYTFTDHAIHWVKQKPEQGLDWIGYISPGNGDIKYNEKFKDKVTLTADKSSSTASMHLNSLTSEDSAVYFCKRSLLALDYWGQGTTLTVSS

SEQ ID NO: 26 - protein, 108 aa, Mus musculus
  source 1..108: note = 3F1 VL domain
DILMTQSHKFMSTSVGDRVSITCKASQDVGTNIAWYQQKPGRSPKVLIYSASTRHTGVPDRFTGSGSGTDFTLTISNVQSEDLTDYFCQQYSSFPLTFGVGTKLELKR

SEQ ID NO: 27 - protein, 116 aa, synthetic construct
  source 1..116: note = VH mutant M1
QVQMQQSDAELVKPGASVKISCKASGYIFADHAIHWVKRKPEQGLEWIGYISPGNDDIKYNEKFKGKATLTADKSSSTAYMQLNSLTSEDSAVYFCKRSLLALDYWGQGTTLTVSS

SEQ ID NO: 28 - protein, 116 aa, synthetic construct
  source 1..116: note = VH mutant M2
QVQMQQSDAELVKPGASVKISCKASGYTFTDHAIHWVKRKPEQGLEWIGYISPGNDDIKYNEKFKGKATLTADKSSSTAYMQLNSLTSEDSAVYFCKRSLLALDYWGQGTTLTVSS

SEQ ID NO: 29 - protein, 116 aa, synthetic construct
  source 1..116: note = VH mutant M3
QVQLQQSDAELVKPGASVKISCKASGYTFTDHAIHWVKRKPEQGLEWIGYISPGNGDIKYNEKFKGKATLTADKSSSTAYMQLNSLTSEDSAVYFCKRSLLALDYWGQGTTLTVSS

SEQ ID NO: 30 - protein, 117 aa, synthetic construct
  source 1..117: note = VH mutant M4
QVQLQQSDAELVKPGASVKISCKASGYTFTDHAIHWVKRKPEQGLEWIGYISPGNGDIKYNEKFKGKATLTADKSSSTAYMQLNSLTSEDSAVYFCKRSLPGTFDYWGQGTTLTVSS

SEQ ID NO: 31 - DNA, 55 bp, synthetic construct
  source 1..55: mol_type = other DNA
ggcggcggcggctccggtggtggtggatcccaagtacagttgcaacagtcagacg

SEQ ID NO: 32 - DNA, 43 bp, synthetic construct
  source 1..43: mol_type = other DNA
cggagtcaggcccccgaggcctgaactgacggtcaaggtcgta

SEQ ID NO: 33 - DNA, 36 bp, synthetic construct
  source 1..36: mol_type = other DNA
catggcggactacaaagacawtgttctcacccagtc

SEQ ID NO: 34 - DNA, 36 bp, synthetic construct
  source 1..36: mol_type = other DNA
catggcggactacaaagacatccagatgacacagwc

SEQ ID NO: 35 - DNA, 36 bp, synthetic construct
  source 1..36: mol_type = other DNA
catggcggactacaaagatrttgtgatgacccagwc

SEQ ID NO: 36 - DNA, 36 bp, synthetic construct
  source 1..36: mol_type = other DNA
catggcggactacaaagacattstgmtgacccagtc

SEQ ID NO: 37 - DNA, 36 bp, synthetic construct
  source 1..36: mol_type = other DNA
catggcggactacaaagatgttgtgvtgacccaaac

SEQ ID NO: 38 - DNA, 36 bp, synthetic construct
  source 1..36: mol_type = other DNA
catggcggactacaaagacacaactgtgacccagtc

SEQ ID NO: 39 - DNA, 36 bp, synthetic construct
  source 1..36: mol_type = other DNA
catggcggactacaaagayattktgctcactcagtc

SEQ ID NO: 40 - DNA, 36 bp, synthetic construct
  source 1..36: mol_type = other DNA
catggcggactacaaagatattgtgatracccaggm

SEQ ID NO: 41 - DNA, 36 bp, synthetic construct
  source 1..36: mol_type = other DNA
catggcggactacaaagacattgtaatgacccaatc

SEQ ID NO: 42 - DNA, 36 bp, synthetic construct
  source 1..36: mol_type = other DNA
catggcggactacaaagacattgtgatgwcacagtc

SEQ ID NO: 43 - DNA, 36 bp, synthetic construct
  source 1..36: mol_type = other DNA
catggcggactacaaagatrtccagatgamccagtc

SEQ ID NO: 44 - DNA, 36 bp, synthetic construct
  source 1..36: mol_type = other DNA
catggcggactacaaagatggagaaacaacacaggc

SEQ ID NO: 45 - DNA, 36 bp, synthetic construct
  source 1..36: mol_type = other DNA
catggcggactacaaagacgctgttgtgactcagga

SEQ ID NO: 46 - DNA, 36 bp, synthetic construct
  source 1..36: mol_type = other DNA
catggcggactacaaagaccytgtgctcactcagtc

SEQ ID NO: 47 - DNA, 65 bp, synthetic construct
  source 1..65: mol_type = other DNA
ggagccgccgccgccagaaccaccaccaccagaaccaccaccaccgcgtttbatttccagcttgg

SEQ ID NO: 48 - DNA, 65 bp, synthetic construct
  source 1..65: mol_type = other DNA
ggagccgccgccgccagaaccaccaccaccagaaccaccaccaccgcgttttatttccaattttg

SEQ ID NO: 49 - DNA, 65 bp, synthetic construct
  source 1..65: mol_type = other DNA
ggagccgccgccgccagaaccaccaccaccagaaccaccaccaccgcgtaggacagtcamcytgg

SEQ ID NO: 50 - DNA, 72 bp, synthetic construct
  source 1..72: mol_type = other DNA
gtatcgtgtagagactgcgtaggtttvvvvttvvvvttvvvvttvvvvtttagccgctggattgttattact

SEQ ID NO: 51 - DNA, 75 bp, synthetic construct
  source 1..75: mol_type = other DNA
agtgatcgagtcagtgcgagtgtttvvvvttvvvvttvvvvttvvvvtttaatcaccggaaccagagccaccacc

SEQ ID NO: 52 - DNA, 60 bp, synthetic construct
  source 1..60: mol_type = other DNA
ggtgctgaagaaagttgtcggtgtctttgtgttaaccgtatcgtgtagagactgcgtagg

SEQ ID NO: 53 - DNA, 59 bp, synthetic construct
  source 1..59: mol_type = other DNA
ggtgctgaagaaagttgtcggtgtctttgtgttaaccagtgatcgagtcagtgcgagtg

SEQ ID NO: 54 - protein, 20 aa, synthetic construct
GGGGSGGGGSGGGGSGGGGS

SEQ ID NO: 55 - protein, 7 aa, synthetic construct
  CARBOHYD 4: note = O-linked (GalNAc)
  CARBOHYD 5: note = O-linked (GalNAc)
APGSTAP

SEQ ID NO: 56 - protein, 7 aa, synthetic construct
  CARBOHYD 3: note = O-linked (GalNac)
  CARBOHYD 4: note = O-linked (GalNac)
GASTGSP

SEQ ID NO: 57 - protein, 24 aa, synthetic construct
  CARBOHYD 11: note = O-linked (GalNAc)
  CARBOHYD 12: note = O-linked (GalNAc)
SAPDTRPAPGSTAPPAHGVTSAPD

SEQ ID NO: 58 - protein, 24 aa, synthetic construct
  CARBOHYD 11: note = O-linked (GalNAc)
SAPDTRPAPGSTAPPAHGVTSAPD

SEQ ID NO: 59 - protein, 24 aa, synthetic construct
  CARBOHYD 12: note = O-linked (GalNAc)
SAPDTRPAPGSTAPPAHGVTSAPD

SEQ ID NO: 60 - protein, 24 aa, synthetic construct
SAPDTRPAPGSTAPPAHGVTSAPD

SEQ ID NO: 61 - protein, 30 aa, synthetic construct
  CARBOHYD 13: note = O-linked (GalNAc)
APPAHGVTSAPDTRPAPGSTAPPAHGVTSA

SEQ ID NO: 62 - protein, 5 aa, synthetic construct
  CARBOHYD 2: note = O-linked (GalNAc)
  CARBOHYD 3: note = O-linked (GalNAc)
GSTAP

SEQ ID NO: 63 - protein, 5 aa, synthetic construct
  CARBOHYD 3: note = O-linked (GalNAc)
GSTAP

SEQ ID NO: 64 - protein, 20 aa, synthetic construct
  CARBOHYD 11: note = O-linked (GalNAc)
  CARBOHYD 12: note = O-linked (GalNAc)
VPSTPPTPSPSTPPTPSPSA

SEQ ID NO: 65 - protein, 21 aa, synthetic construct
  CARBOHYD 9: note = O-linked (GalNAc)
  CARBOHYD 10: note = O-linked (GalNAc)
PLWTSIGASTGSPLPPEPTTY

SEQ ID NO: 66 - protein, 21 aa, synthetic construct
PLWTSIGASTGSPLPPEPTTY

SEQ ID NO: 67 - protein, 5 aa, synthetic construct
  CARBOHYD 2: note = O-linked (Neu5Ac(a2-6)GalNAc)
  CARBOHYD 3: note = O-linked (Neu5Ac(a2-6)GalNAc)
GSTAP

SEQ ID NO: 68 - protein, 5 aa, synthetic construct
  CARBOHYD 3: note = O-linked (Neu5Ac(a2-6)GalNAc)
GSTAP

SEQ ID NO: 69 - protein, 20 aa, synthetic construct
  CARBOHYD 13: note = O-linked (GALNAc)
  CARBOHYD 14: note = O-linked (GalNAc)
VTSAPDTRAAPGSTAPPAHG

SEQ ID NO: 70 - protein, 20 aa, synthetic construct
  CARBOHYD 13: note = O-linked (GalNAc)
  CARBOHYD 14: note = O-linked (GalNAc)
VTSAPDTRPAAGSTAPPAHG

SEQ ID NO: 71 - protein, 20 aa, synthetic construct
  CARBOHYD 13: note = O-linked (GalNAc)
  CARBOHYD 14: note = O-linked (GalNAc)
VTSAPDTRPAPASTAPPAHG

SEQ ID NO: 72 - protein, 20 aa, synthetic construct
  CARBOHYD 14: note = O-linked (GalNAc)
VTSAPDTRPAPGATAPPAHG

SEQ ID NO: 73 - protein, 20 aa, synthetic construct
  CARBOHYD 13: note = O-linked (GalNAc)
VTSAPDTRPAPGSAAPPAHG

SEQ ID NO: 74 - protein, 20 aa, synthetic construct
  CARBOHYD 13: note = O-linked (GalNAc)
  CARBOHYD 14: note = O-linked (GalNAc)
VTSAPDTRPAPGSTAAPAHG

SEQ ID NO: 75 - protein, 20 aa, synthetic construct
  CARBOHYD 13: note = O-linked (GalNAc)
  CARBOHYD 14: note = O-linked (GalNAc)
VTSAPDTRPAPGSTAPAAHG

SEQ ID NO: 76 - protein, 20 aa, synthetic construct
  CARBOHYD 13: note = O-linked (GalNAc)
  CARBOHYD 14: note = O-linked (GalNAc)
VTSAPDTRPAPGSTAPPAAG

SEQ ID NO: 77 - protein, 20 aa, synthetic construct
  CARBOHYD 13: note = O-linked (GalNAc)
  CARBOHYD 14: note = O-linked (GalNAc)
APGSTAPPAHGVTSAPDTRP

SEQ ID NO: 78 - protein, 20 aa, synthetic construct
  CARBOHYD 14: note = O-linked (GalNAc)
VTSAPDTRPAPGSTAPPAHG

SEQ ID NO: 79 - protein, 9 aa, Mus musculus
QQYNSYPLT

SEQ ID NO: 80 - protein, 8 aa, Mus musculus
QQYYSYPT

SEQ ID NO: 81 - protein, 9 aa, Mus musculus
QQYSSYPLT

SEQ ID NO: 82 - protein, 9 aa, Mus musculus
QQYYSYPLT

SEQ ID NO: 83 - protein, 9 aa, Mus musculus
QQYNSYPLA

SEQ ID NO: 84 - protein, 9 aa, Mus musculus
HQHYSTPPT

SEQ ID NO: 85 - protein, 9 aa, Mus musculus
KQYNSYPLT

SEQ ID NO: 86 - protein, 9 aa, Mus musculus
LQHWNYPLT

SEQ ID NO: 87 - protein, 9 aa, Mus musculus
QQDYSSPLT

SEQ ID NO: 88 - protein, 9 aa, Mus musculus
QQYNSFPLA

SEQ ID NO: 89 - protein, 10 aa, Mus musculus
QNDYSYPLTF

SEQ ID NO: 90 - protein, 10 aa, Mus musculus
QQYYSYPLTF

SEQ ID NO: 91 - protein, 10 aa, Mus musculus
QQYYSYPLTF

SEQ ID NO: 92 - protein, 10 aa, Mus musculus
QQYYSYPLTF

SEQ ID NO: 93 - protein, 10 aa, Mus musculus
LQHWNYPLTF

SEQ ID NO: 94 - protein, 10 aa, Mus musculus
LQHWNYPLTF

SEQ ID NO: 95 - protein, 10 aa, Mus musculus
QQYSSYPLTF

SEQ ID NO: 96 - protein, 10 aa, Mus musculus
QQYSSYPLTF

SEQ ID NO: 97 - protein, 11 aa, Mus musculus
KASQDVSTAVA

SEQ ID NO: 98 - protein, 7 aa, Mus musculus
SASYRYT

SEQ ID NO: 99 - protein, 9 aa, Mus musculus
QQYNSYPYT

SEQ ID NO: 100 - protein, 11 aa, Mus musculus
KASQDVSTAVA

SEQ ID NO: 101 - protein, 7 aa, Mus musculus
SASYRYS

SEQ ID NO: 102 - protein, 9 aa, Mus musculus
QQYNSYPLT

SEQ ID NO: 103 - protein, 11 aa, Mus musculus
KASQDVSTAVA

SEQ ID NO: 104 - protein, 7 aa, Mus musculus
SASYRYT

SEQ ID NO: 105 - protein, 9 aa, Mus musculus
QQHYSTPYT

SEQ ID NO: 106 - protein, 11 aa, Mus musculus
KASQDVGTAVA

SEQ ID NO: 107 - protein, 7 aa, Mus musculus
WASTRHT

SEQ ID NO: 108 - protein, 9 aa, Mus musculus
QQYSSYPYT

SEQ ID NO: 109 - protein, 11 aa, Mus musculus
KASQDVGTAVA

SEQ ID NO: 110 - protein, 7 aa, Mus musculus
WASTRHT

SEQ ID NO: 111 - protein, 9 aa, Mus musculus
QQYNSYPYT

SEQ ID NO: 112 - protein, 11 aa, Mus musculus
KASQDVSTAVA

SEQ ID NO: 113 - protein, 7 aa, Mus musculus
SASYRYS

SEQ ID NO: 114 - protein, 9 aa, Mus musculus
QQYNSYPYT

SEQ ID NO: 115 - protein, 11 aa, Mus musculus
KASQNVGTAVA

SEQ ID NO: 116 - protein, 7 aa, Mus musculus
SASNRYT

SEQ ID NO: 117 - protein, 9 aa, Mus musculus
QQYSSYPYT

SEQ ID NO: 118 - protein, 11 aa, Mus musculus
KASQDVSTAVA

SEQ ID NO: 119 - protein, 7 aa, Mus musculus
SASYRYT

SEQ ID NO: 120 - protein, 9 aa, Mus musculus
QQYNSYPLT

SEQ ID NO: 121 - protein, 11 aa, Mus musculus
KASQNVGTNVA

SEQ ID NO: 122 - protein, 7 aa, Mus musculus
SASYRYS

SEQ ID NO: 123 - protein, 9 aa, Mus musculus
QQYNSYPYT

SEQ ID NO: 124 - protein, 11 aa, Mus musculus
KASQDVGTAVA

SEQ ID NO: 125 - protein, 7 aa, Mus musculus
WASTRHT

SEQ ID NO: 126 - protein, 9 aa, Mus musculus
QQYSSYPLT

SEQ ID NO: 127 - protein, 10 aa, Mus musculus
QHHYGTPYTF

SEQ ID NO: 128 - protein, 10 aa, Mus musculus
QQHYSTPYTF

SEQ ID NO: 129 - protein, 10 aa, Mus musculus
QQYNSYPLTF

SEQ ID NO: 130 - protein, 10 aa, Mus musculus
QQYYSYPWTF

SEQ ID NO: 131 - protein, 10 aa, Mus musculus
QHFWSTPYTF

SEQ ID NO: 132 - protein, 10 aa, Mus musculus
QQYYSYPYTF

SEQ ID NO: 133 - protein, 10 aa, Mus musculus
QQYYSYPWTF

SEQ ID NO: 134 - protein, 10 aa, Mus musculus
QQYYSYPYTF

SEQ ID NO: 135 - protein, 9 aa, Mus musculus
QQYNSYPYT

SEQ ID NO: 136 - protein, 9 aa, Mus musculus
QQYYSYPRT

SEQ ID NO: 137 - protein, 9 aa, Mus musculus
QQHYSTPRT

SEQ ID NO: 138 - protein, 9 aa, Mus musculus
SQSTHVPRT

SEQ ID NO: 139 - protein, 9 aa, Mus musculus
QQYYSYPWT

SEQ ID NO: 140 - protein, 9 aa, Mus musculus
QQHYSTPWT

SEQ ID NO: 141 - protein, 9 aa, Mus musculus
HQYHRSPPT

SEQ ID NO: 142 - protein, 9 aa, Mus musculus
HQYHRSPRT

SEQ ID NO: 143 - protein, 9 aa, Mus musculus
QQRSSYPYT

SEQ ID NO: 144 - protein, 9 aa, Mus musculus
QQHYSTPYT

SEQ ID NO: 145 - protein, 9 aa, Mus musculus
QQHYSTPRT

SEQ ID NO: 146 - protein, 9 aa, Mus musculus
QQYNSYPYT

SEQ ID NO: 147 - protein, 9 aa, Mus musculus
QQYYSYPRT

SEQ ID NO: 148 - protein, 9 aa, Mus musculus
KQYHRSPPT

SEQ ID NO: 149 - protein, 9 aa, Mus musculus
HQYHRSPPT

SEQ ID NO: 150 - protein, 9 aa, Mus musculus
QQYYSYPPT

SEQ ID NO: 151 - protein, 9 aa, Mus musculus
QQWSSNPYT

SEQ ID NO: 152 - protein, 9 aa, Mus musculus
QQHYSTPWT

SEQ ID NO: 153 - protein, 9 aa, Mus musculus
SQSTHVPRT

SEQ ID NO: 154 - protein, 9 aa, Mus musculus
HQYHRSPRT

(19) United States
(12) Patent Application Publication
(10) Pub. No.: US 2024/0309110 A1
(43) Pub. Date: Sep. 19, 2024

(54) Combotope Antibody Libraries

(71) Applicants: DANMARKS TEKNISKE UNIVERSITET, Kongens Lyngby (DK); UNIVERSIDAD DE ZARAGOZA, Zaragoza (ES); Fundación Agencia Aragonesa para la Investigación y el Desarrollo (ARAID), Zaragoza (ES)

(72) Inventors: Ola Blixt, Kongens Lyngby (DK); Ramón Hurtado Guerrero, Zaragoza (ES); Spyridon Gatos, Kongens Lyngby (DK)

(21) Appl. No.: 18/526,205

(22) Filed: Dec. 1, 2023

Related U.S. Application Data
(63) Continuation-in-part of application No. PCT/EP23/56922, filed on Mar. 17, 2023.

(51) Int. Cl. C07K 16/30; A61K 49/00; C07K 16/28; C12N 15/10

(52) U.S. Cl. CPC C07K 16/3092; A61K 49/0002; C07K 16/2896; C12N 15/1037; A61K 39/00; C07K 2317/24; C07K 2317/622

(57) ABSTRACT

The invention provides specific Tn- and/or STn antibody libraries and methods for identifying specific antibodies, which target Tn- and/or STn-glycosylation sites of any glycoprotein of choice, especially glycoproteins of cancer cell targets. The invention further provides antibodies identified by the new concept proposed herein, which have combined specificity towards both the sugar epitope as well as the peptide epitope of the glycoprotein.

### CROSS-REFERENCE TO RELATED APPLICATIONS

This application is a continuation-in-part of International Application No. PCT/EP2023/056922, filed Mar. 17, 2023, the content of which is herein incorporated by reference in its entirety.

### SEQUENCE LISTING STATEMENT

The contents of the electronic sequence listing titled P3311PC00_ST26.xml (Size: 156,091 bytes; and Date of Creation: Dec. 1, 2023) is herein incorporated by reference in its entirety.

### FIELD OF THE INVENTION

The invention provides specific Tn- and/or STn antibody libraries and methods for identifying specific antibodies, which target Tn- and/or STn-glycosylation sites of any glycoprotein of choice, especially relevant for binding to cancer cell targets. The invention further provides antibodies identified by the new concept proposed herein, which have combined specificity towards both the sugar epitope as well as the peptide epitope of the glycoprotein.

### BACKGROUND

A dense layer of complex carbohydrate structures covers almost all eukaryotic cells. Tumor cells, contrary to their healthy counterparts, exhibit altered glycosylation patterns on the cell surface. Such altered cancer glycosylation includes increased sialylation, fucosylation, short truncated O-glycans and increased N-branching.

One of the key features of glycosylation changes in cancer is the short truncated O-glycans, the so-called tumor-associated carbohydrate antigens (TACAs): Tn and T antigens and the sialylated forms of them, STn and ST, respectively.

The Tn and STn are not commonly observed in any normal human or rodent tissues, but are highly expressed on many solid tumors/carcinomas. Thus, Tn and STn represent major targets of potential immunotherapy as well as being useful in diagnostic of cancereous states.

There are a number of cell surface glycoproteins with altered glycosylation that play key roles in cancer including MUC1, MUC4 and MUC6 from the mucin family along with CD43 and CD44 as examples.

MUC1 is the most well-studied mucin from the mucin family and is present in many adenocarcinomas displaying short truncated O-glycans. Under healthy conditions, MUC1 peptide core is heavily glycosylated and therefore masked by the O-glycan moieties that protect MUC1 from proteolytic cleavage enzymes. In adenocarcinomas, MUC1 proteins have shorter and less dense O-glycan side chains, resulting in exposure of the core domains of the protein on the cell surface. This altered glycosylation on MUC1 results in exposure of the epitopes MUC1-Tn and MUC1-STn to the immune system.

CD43 (leukosialin) is a type I transmembrane sialoglycoprotein that is abundant in hematopoietic cells, including lymphocytes, monocytes, granulocytes, natural killer cells, platelets except resting mature B cells, and erythrocytes. The human CD43 protein has a mucin-type extracellular domain rich in serine and threonine residues enabling extensive O-GalNAc glycosylation with significant molecular weight heterogeneity. CD43 glycoforms have been reported in several hematological and non-hematopoietic cancers, including the lung, breast, colon, cervix, and prostate, which express CD43 mostly in the early stages of tumor progression.

Known antibodies related to Tn/STn include 5E5 (Macias-Leon et al 2020; Tarp et al 2007; Blixt et al 2010), anti-CD43 (Blixt et al 2012), 2D9 (Sorensen et al 2006; Tarp et al 2007; Blixt et al 2010), G2D11 (Persson et al 2017), 3F1 (Kjeldsen et al 1988), 83D4 (Oppezzo et al 2004), 15G9 (Mazal et al 2013), 1E3 (Li et al 2009), and MLS128 (Yuasa et al 2012); 16E12.1D9.1B11 (WO 2023/034569 A1) and 1 A5-2C9 (US 2022/057402 A1). These antibodes are Tn-hapten binders with no or unknown binding contribution to the peptide/protein carrier, as will be further discussed herein.

Monoclonal antibodies to the Tn and STn antigens are notably difficult to generate and are expensive to produce, and their specificities are often not well characterized, especially in regard to whether these antibodies simultaneously recognize the Tn/STn and the protein backbone/carrier, a requirement for superior specificity and therapeutic use.

The development of therapeutic antibodies can be achieved via several strategies. Current technologies, such as the well-established method of hybridoma technology, rely on animal immunization, where the animals are challenged with the antigen of interest to elicit an immune response. Then, B cells from the spleen of immunized animals (mice, rat and rabbit) are isolated and the subsequent fusion with myeloma cells to produce hybridoma clones for testing. It is a very tedious, laborious, time-consuming method and many times the fusion efficiency is low. Also, the effectiveness of the method relies largely on the immunogenicity of the antigen, which some antigen shown to be poorly immunogenic.

Phage display facilitates expression of proteins on the surface of phages. It is a molecular technique using the filamentous phage, where foreign DNA, encoding a peptide, is inserted in nonlytic philamentous phage genome and expressed as a fusion protein together with the phage coat protein without affecting the phage infectivity. Phage display allows a large repertoire of antibodies or parts thereof to be displayed on phages for selection of high affinity binders against a target of interest.

### SUMMARY OF THE INVENTION

The present invention provides a new antibody concept technology for rapid development of combotope antibodies (Abs) targeting Tn- and STn-glycosylation site of any glycoprotein site of choice, paving the way for a new generation of therapeutic opportunities in cancer treatment and diagnostics.

A novel structural and biochemical explanation, provided herein for the first time, of Tn- and STn- recognition specifically by the VH hypervariable region of the antibody, is combined with phage display screening for specific peptide recognition by the VL domains, thereby providing combotope antibodies with high specificity and affinity to desired biological glycoprotein targets due to the combined recognition of the Tn and/or STn carbohydrate epitope and the protein backbone/carrier associated with the Tn and/or STn carbohydrate epitope.

Phage display is a fast method for antibody development. The constructed Tn and STn template libraries define the VH part of the antibody binding the desired glycoform, and the VL diversity will determine the peptide backbone specificity. As disclosed herein in the examples section, MUC1 was used as a proof of concept target for both libraries to evaluate the functionality of the two libraries. scFvs with sequences being the same as or similar to the already known MUC1 antibodies were isolated from biopanning of the libraries. CD43 was further the first target that was used to identify scFvs against Tn-CD43 peptide. Both Tn-MUC1 and Tn-CD43 scFvs were characterized for their specificity in vitro, and the scFvs demonstrated their potential uses as therapeutics on cancer cell lines. Furthermore, STn-MUC1 scFvs were identified.

In that way, two template libraries have been constructed, a Tn-template library (see examples 2-4) and a STn-template library (see examples 5-6), where the VH domain pre-selects for the desired glycoform specificity, and the VL domain will determine the peptide backbone specificity. The constructed libraries are the first libraries to be specifically designed for glycosylated targets.

In a first aspect, the invention provides an antibody library for in-vitro identification of a specific antibody which binds a tumor cell, wherein each antibody in said library comprises
(i) a first antibody domain which binds a Tn- and/or STn-carbohydrate epitope of a glycoprotein of said tumor cell, and
(ii) a second antibody domain selected from a repertoire of second antibody domains, wherein the repertoire of second antibody domains comprises one or more second antibody domains which binds a peptide epitope of said glycoprotein of said tumor cell,

wherein said specific antibody is specific for a combination of said carbohydrate epitope and said peptide epitope of said glycoprotein.

In one preferred embodiment, the first antibody domain is a VH-domain and the second antibody domain is a VL-domain. Hence, In a preferred embodiment, the invention provides an antibody library, wherein each antibody in the library comprises
(i) a VH-domain which binds a Tn- and/or STn-carbohydrate epitope of a glycoprotein of said tumor cell, and
(ii) a VL-domain selected from a repertoire of VL-domains, wherein the repertoire of VL-domains comprises one or more VL-domains which binds a peptide epitope of said glycoprotein of said tumor cell,

for in-vitro identification of a specific antibody from said library which binds a tumor cell; wherein the specific antibody is specific for a combination of said carbohydrate epitope and said peptide epitope of said glycoprotein.

In a second aspect, the invention provides a nucleic acid library encoding the antibody library of the first aspect of the invention.

In a third aspect, the invention provides a method for identifying an antibody for targeting a tumor cell, comprising the steps of
i) preparing an antibody library according to the first aspect of the invention, and
ii) screening said library to identify one or more tumor targeting antibodies.

In a fourth aspect, the invention provides a method for identifying a glycopeptide target, said target comprising a Tn and/or STn epitope and a peptide epitope, such as a glycopeptide target of a cancer cell, said method comprising the steps of
i) preparing an antibody library according to the first aspect of the invention, and
ii) incubating the antibody library with a sample comprising the glycopeptide target,
iii) analyzing one or more antibody-peptide complexes obtained from step ii) to identify the amino acid sequence of the peptide epitope of said glycopeptide target.

In a sixth aspect, the invention provides a specific tumor cell binding antibody, comprising
(i) a VH-domain which binds a Tn- and/or STn-carbohydrate epitope of a glycoprotein of the tumor cell, and
(ii) a VL-domain which binds a peptide epitope of said glycoprotein of said tumor cell,

wherein the antibody is specific for a combination of said carbohydrate epitope and said peptide epitope of said glycoprotein,

with the provisio that the antibody is not 5E5, 5F7 or 2D9.

Preferably, the antibodies may be used in method of treatment and/or prevention of cancer.

### DESCRIPTION OF THE INVENTION

### Definitions and Abbreviations

Unless specifically stated, as used herein, the term “nucleic acid” encompasses double as well as single-stranded nucleotide molecules. Nucleic acid sequences, when provided, are listed in the 5′ to 3′ direction, unless stated otherwise.

The term “homology”, “similarity” or “sequence identity” between two proteins is determined by comparing the amino acid sequence and its conserved amino acid substitutes of one protein sequence to the second protein sequence. Similarity may be determined by procedures which are well-known in the art, for example, a BLAST program (Basic Local Alignment Search Tool at the National Center for Biological Information). Likewise, homology, similarity or sequence identity between two nucleic acid sequences may be determined by procedures which are well-known in the art, for example, a BLAST program (Basic Local Alignment Search Tool at the National Center for Biological Information).

“Amino acid residue substitution” at a specific position means substitution with any amino acid different from the native amino acid residue that is present at that specific position. A conservative amino acid substitution replaces an amino acid with another amino acid that is similar in size and chemical properties such that the substitution has no or only minor effect on protein structure and function; meanwhile a nonconservative amino acid substitution replaces an amino acid with another amino acid that is dissimilar and thereby is likely to affect structure and function of the protein.

As used herein, the term “antibody” will be understood to include proteins having the characteristic two-armed, Y-shape of a typical antibody molecule as well as one or more fragments of an antibody that retain the ability to specifically bind to an antigen. Exemplary antibodies include, but are not limited to, a monoclonal antibody, a polyclonal antibody, a bi-specific antibody, a multispecific antibody, a grafted antibody, a human antibody, a humanized antibody, a synthetic antibody, a chimeric antibody, a camelized antibody, a single-chain Fvs (scFv) (including fragments in which the VL and VH are joined using recombinant methods by a synthetic or natural linker that enables them to be made as a single protein chain in which the VL and VH regions pair to form monovalent molecules, including single chain Fab and scFab), a single chain antibody, a Fab fragment (including monovalent fragments comprising the VL, VH, CL, and CHI domains), aF(ab′)2 fragment (including bivalent fragments comprising two Fab fragments linked by a disulfide bridge at the hinge region), a Fd fragment (including fragments comprising the VH and CHI fragment), a Fv fragment (including fragments comprising the VL and VH domains of a single arm of an antibody), a single-domain antibody (dAb or sdAb) (including fragments comprising a VH domain), an isolated complementarity determining region (CDR), a diabody (including fragments comprising bivalent dimers such as two VL and VH domains bound to each other and recognizing two different antigens), a fragment comprised of only a single monomeric variable domain, disulfide-linked Fvs (sdFv), an intrabody, an anti-idiotypic (anti-Id) antibody, or ab antigen-binding fragments thereof.

As recognized by a person skilled in the art, the term VL refers to antibody variable domain, light chain, and the term VH refers to antibody variable domain, heavy chain.

The term “Tn antigen” as used herein refers to the monosaccharide structure N-acetylgalactosamine (GalNAc) linked to serine (Ser) or threonine (Thr) on a peptide backbone by a glycosidic bond (i.e. GalNAcα1-O-Ser/Thr). The initials stand for Thomsen-nouveau. Tn antigen is expressed in most carcinomas. The term “Tn-carbohydrate epitope” (or “Tn epitope”) as used herein refers to the GalNac part of the Tn antigen. The term “mono-Sn” refers to one Tn moiety (i.e. one GalNAc). The term “bis-Tn” refers to two Tn moieties (i.e. two GalNAc).

The term “STn antigen” as used herein refers to a sialyl-Tn antigen, formed by elongation of the Tn antigen with sialic acid (Neu5Ac(a2-6)GalNAc), still linked to serine (Ser) or threonine (Thr) (i.e. Neu5Acα2-6GalNAcα1-O-Ser/Thr). Such STn antigen is also common on cancer tumor cells. Both Tn and STn may have additional modifications, such as phosphorylation, acetylation, methylation, and sulfonation. The term “STn-carbohydrate epitope” (or “STn epitope”) as used herein refers to the Neu5Ac(a2-6)GalNAc part of the Tn antigen. The term “mono-STn” refers to one STn moiety (i.e. one Neu5Ac(a2-6)GalNAc). The term “bis-STn” refers to two STn moieties (i.e. two Neu5Ac(a2-6)GalNAc).

The term glycoprotein generally refers to proteins which contain oligosaccharide chains covalently attached to amino acid side-chains. The term “glycoprotein” as used herein refers to a protein or peptide which contains a carbohydrate moiety (preferably a Tn or STn epitope) covalently attached to an amino acid residue (such as serine, threonine, or tyrosine; or any non-natural amino acid derivatives thereof such as replacing O with S) of said protein or peptide.

The term “combotope” as used herein refers to the combination of a carbohydrate epitope and a peptide epitope being recognized by an antibody. The two epitopes form a common epitope, the “combotope”, which is different from each of the two epitopes from which it is composed. Of particular relevance for the present invention are combotopes where the peptide epitope is associated with a Tn epitope or a STn epitope; the peptide epitope may be associated with one or two Tn or STn epitopes. The combotope may be contiguous or discontiguous—i.e. the carbohydrate epitope is directly attached to the peptide epitope by covalent bond (contiguous), or the carbohydrate epitope is attached to an amino acid residue located 1, 2, 3, 4, 5, 6, 7, 8, 9, or 10 amino acid residues up or down stream of the peptide epitope (discontiguous). Preferably, the carbohydrate epitope is covalently attached to the peptide epitope.

The carbohydrate epitope may also be a combination of two Tn or STn epitopes (termed bis-Tn and bis-STn) or one Tn and one STn epitope, on adjacent amino acids in said peptide sequence in said glycoprotein. Adjacent means separated by 0 to 2 amino acids.

Antibodies which are refered to as “combotope binder” recognize and bind the combination of both the carbohydrate epitope and a peptide epitope of the glycoprotein. Such antibodies are also referred to as “combotope antibodies”.

In the present application, the term “hapten” refers to the carbohydrate moiety(ies) (Tn or STn) independent of the peptide/protein. Antibodies which are referred to as “hapten binders” will bind Tn or STn epitopes independent of the peptide/protein carries—hence, they are therefore not specific for the combination of both the carbohydrate epitope and a peptide epitope of the glycoprotein (i.e. not a combotope binder).

The phase “domain which binds . . . ” as used herein should be understood as “domain which is suitable for binding . . . ”, “domain which is capable of binding . . . ”, and/or “domain which is prepared for binding . . . ”.

### DESCRIPTION OF FIGURES

FIG. 1: Schematic illustration of an embodiment of the invention: Tn-template antibody library for identifying Tn-combotope antibodies. Phage display of a library of scFv antibodies. Each scFv in the library has a Tn-binding VH domain. The library is screened for Tn-peptide specific scFv by biopanning using Tn-peptides.

FIG. 2: Illustration of the preparation of a phage display library of the present invention. 1) mRNA isolation from mouse spleen. 2) cDNA synthesis with reverse transcriptase using random hexamers. 3) PCR amplification from cDNA template to obtain the VH domain using a specific set primers, and the repertoire of VL domains using a mix of VL primers. 4) PCR assembly of VL-domain repertoire and specific VH-domain using 5′ phosphorylated outer primers. 5) Rolling circle amplification where phosphorylated scFv genes are ligated into circular DNA, dsDNA is denatured, random hexamers are annealed and Phi29 polymerase amplifies the circular fragments into long linear concatemers. 6) Amplified extended scFv genes are digested by sfiI restriction enzyme and ligated to Sfil and rSAP treated phagemid vector pAK100. 7) Pool of phagemids containing scFv genes are electroporated to TG1 E. coli cells. 8) Growth of Bacterial Library containing the different phagemids and infection with helper phage VCSM13 to produce complete phages displaying scFvs on their pIII coat protein

FIG. 3: X-ray of G2D11 scFv with APGS*T*AP peptide (where * denotes a GalNac residue) showed the interaction points of VH with the glycan structure. Key interaction points with two adjacent GalNac residues include His32H, Ala33H, His35H in CDR1; His40^{H}, Ser52^{H}, Asn55^{H}, Asp57^{H} in CDR2; and Ser99^{H} in CRD3.

FIG. 4: VH domain sequence alignment of G2D11 with other known VH-domains. Conserved amino acid residues are indicated by arrows (H32, A33, H35, Y50, S52, N55, D57 AND S99)

FIG. 5: Phage and sequence enrichment after each round (Round 1, 2, and 3) of biopanning for bisTn-MUC1. Polyclonal phage ELISA confirmed phage enrichments for bis Tn-MUC1 target peptide. bisTn MUC1=peptide no 1 in Table 1; Tn MUC1=peptide no 3 in Table 1; SA=negative control.

FIG. 6. Confirmation of MUC1 scFv and selected clones for expression by dot blot analysis of scFv overnight expression in the 96 well format by His-tag detection.

FIG. 7. MUC1 monoclonal ELISA. Ccreening of monoclonal scFvs on MUC1 target and control peptides.

FIG. 8. MUC1 scFvs binding assays and kinetic affinities. (A) scFv titration at fixed concentration of MUC1 target peptide (peptide no 1 in Table 1). (B) scFv titration at fixed concentration of IgA hinge region control peptide (peptide no 8). Each data point is the mean value of three independent experiments. (C) Representative histograms of cell binding at 1.25 μg/ml of A3, D2 and D3 scFvs along with 5E5 mAb on MDA-MB-231 WT and COSMC KO cells. Flow cytometry experiments were repeated three times.

FIG. 9. MUC1 scFv titration on MUC1 peptides 2, 3, 4 and 5. MUC1 scFvs titrated on different MUC1 glycopeptides and unglycosylated MUC1. Asterisk denotes a glycosylation site.

FIG. 10. MUC1 scFvs biological evaluation with flow cytometry. (A) Negative binding of MUC1 scFvs on HEK293 cells as a negative control cell line. (B) MCF7 cells at 1.25 μg/mL. C) Representative example of concentration dependent binding on MDA-MB-231 WT and COSMC KO cells. scFv D3 is shown at 4-fold dilution starting from 5 μg/mL.

FIG. 11. X-ray of 5E5 scFv with APGST*AP peptide (asterisk denotes a GalNac residue). Tyr98L and the conserved Tyr100L are the key amino acids in recognition of the peptide backbone.

FIG. 12. Heat map of binding of scFv D3, scFv A4, scFv 5E5, scFv 2D9Chi, and scFv G2D11 to Tn-glycopeptides from Table 5. The glycopeptides were printed on a microarray chip. For simplicity, the heat map shows amino acids 9-19 of the peptides in Table 5. The relative fluorescence units (RFU) as shown as heat map. Tn-glycosylation sites are bold and underlined. Substitutions with Ala are marked as bold.

FIG. 13. Polyclonal phage enrichment between the three rounds of selection against bisTn-CD43 target peptide (peptide no. 9 in Table 1) and control peptides (peptide no. 10 in Table 1).

FIG. 14. CD43 monoclonal ELISA. Screening of monoclonal scFvs on CD43 target and control peptides.

FIG. 15. CD43 scFvs binding assays. (A) Eight scFvs were were titrated on bisTn-CD43 target peptide (peptide no. 9 in Table 1). (B) ScFv titration on IgA1 hinge region control glycopeptide (peptide no. 8 in Table 1) showed A7, D3 cross reactivity to IgA while A1 and F4 showed weaker binding to IgA1. Each dot represents the mean value of three independent experiments. (C) Representative histograms of A1, D7, H1 and H2 scFvs at 1.25 μg/mL tested on Jurkat cells before and after neuraminidase treatment. Flow cytometry experiments were repeated three times.

FIG. 16. CD43 scFvs biological evaluation with flow cytometry. Concentration dependent binding of A1 scFv as a representative example on HEK293 cells and Jurkat cells, before and after neuraminidase treatment. scFv was 4-fold diluted starting from 5 μg/mL.

FIG. 17. CD43 x-ray structure with GAS*T*GSP peptide reveals the importance of Tyr99L as key interaction point with the peptide backbone.

FIG. 18. Alignment of bisTn binder G2D11 VH with monoTn binder 3F1 VH to identify amino acid residues relevant for shifting to anti bisSTn.

FIG. 19. Microarray data for binding of G2D11, 3F1, and mutants (M1-4) comprising selected mutations of VH-G2D11 to glycopeptides 1 (bisTnMUC1), 11 (bisSTnMUC1), 12 (monoSTnMUC1) and 4 (unglycosylated control).

FIG. 20. Microarray data for binding of STnMUC1-D4, D3, C7 scFv to glycopeptides 1 (bisTnMUC1), 11 (bisSTnMUC1), and 12 (monoSTnMUC1).

### DETAILED DESCRIPTION OF THE INVENTION

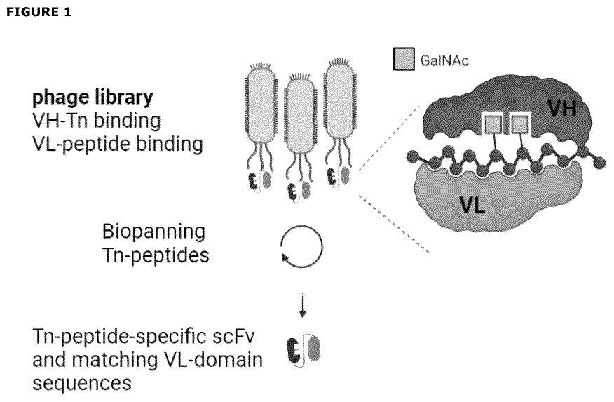

The present invention concerns a new antibody concept technology for simple and rapid development of antibodies targeting Tn- and STn-glycosylation sites of any glycoprotein site of choice. Specifically, the invention concerns antibody libraries which can be screened for antibodies which have improved specificity due to their specificity towards a combination of an epitopes on a carbohydrate part of a glycoprotein and an epitope on a peptide backbone in said glycoprotein which is associated with the carbohydrate epitope. The combined epitope is termed a “combotope” A non-limiting embodiment of the invention is schematically illustrated in FIG. 1.

The present invention is especially useful in the generation of therapeutics for cancer treatment and diagnostics.

### I. Combotope Antibodies

The present invention provides combotope antibodies which have high specificity and high binding efficiency to their target glycopeptide due to their combined specificity towards both the carbohydrate epitope as well as the peptide backbone epitope associated with the carbohydrate epitope of the glycoprotein target. In one preferred embodiment, the present invention provides a combotope antibody for targeting tumor cells carrying said glycoprotein on their surface.

In one aspect, the invention provides an antibody for targeting tumor cells, said antibody comprising two antibody domains, where the first antibody domain binds a carbohydrate epitope of a glycoprotein of the tumor cell and the second antibody domain binds a peptide epitope of said glycoprotein of said tumor cell, where said antibody specifically binds both epitopes (as a common epitope) as compared to only binding one of said epitopes.

In one embodiment, the first antibody domain is a VH domain and the second antibody domain is a VL domain. In another embodiment, both the first and second antibody are VH domains, but different from one another.

In one aspect, the invention provides an antibody for targeting tumor cells, said antibody comprising (i) a VH domain binding a carbohydrate epitope of a glycoprotein of the tumor cell and (ii) a VL domain binding a peptide epitope of said glycoprotein of said tumor cell, where said antibody is selected to specifically binding both epitopes (as a common epitope) as compared to only binding one of said epitopes.

In one embodiment, the invention provides an antibody for targeting a glycoprotein, such as a glycoprotein on a tumor cell. The glycoprotein comprises a carbohydrate epitope and a peptide epitope. The peptide epitope is associated with the carbohydrate epitope, such as directly attached by virtue of being chemically linked or being in close proximity of one another by virtue of the structural configuration of the glycoprotein. The antibody of the present invention is characterized by its ability to specifically bind both epitopes (as a common epitope), as compared to only binding one of said epitopes. In one embodiment, the antibody comprises (i) a VH domain characterized by (a) being suitable for binding a carbohydrate epitope of a glycoprotein of a tumor cell and (b) not binding the peptide epitope of said glycoprotein of said tumor cell and (ii) a VL domain characterized by being suitable for binding the peptide epitope of said glycoprotein of said tumor cell. In one embodiment, the VL-domain is further characterized by not binding the carbohydrate epitope of said glycoprotein.

In one embodiment, the present invention provides an antibody which binds a tumor cell, wherein the antibody comprises
(i) a VH-domain which binds a carbohydrate epitope of a glycoprotein of a tumor cell, and
(ii) a VL-domain which binds a peptide epitope of said glycoprotein of said tumor cell,

wherein the peptide epitope of the glycoprotein of the tumor cell is associated with the carbohydrate epitope of said glycoprotein of said tumor cells, and wherein the antibody is specific for the combination of said carbohydrate epitope and said peptide epitope on said glycoprotein of said cancer cell.

In one embodiment, the present invention provides an antibody which binds a tumor cell, wherein the antibody comprises (ii) a VL-domain which binds a peptide epitope of a glycoprotein of said tumor cell and (i) a VH-domain which binds a carbohydrate epitope of said glycoprotein of said tumor cell and which does not contribute to or interfere with binding said peptide epitope of said glycoprotein of said tumor cell, i.e. the VH-domain binding is not affected/influenced by the presence of any peptide epitope; and wherein the peptide epitope of the glycoprotein of the tumor cell is associated with the carbohydrate epitope of said glycoprotein of said tumor cells, and wherein the antibody is specific for the combination of said carbohydrate epitope and said peptide epitope on said glycoprotein of said cancer cell.

In one embodiment, the present invention provides an antibody which binds a tumor cell, wherein the antibody comprises (ii) a VL-domain which binds a peptide epitope of a glycoprotein of said tumor cell and (i) a VH-domain which exclusively binds a carbohydrate epitope of said glycoprotein of said tumor cell, i.e. which does not contribute to or interfere with binding peptide epitope of said glycoprotein of said tumor cell; and wherein the peptide epitope of the glycoprotein of the tumor cell is associated with the carbohydrate epitope of said glycoprotein of said tumor cells, and wherein the antibody is specific for the combination of said carbohydrate epitope and said peptide epitope on said glycoprotein of said cancer cell.

As disclosed here, the antibody of the present invention is specific for the combination of the carbohydrate epitope and the peptide epitope of a glycopeptide of a tumor cell, such on the surface of the tumor cell. The term “specific” in this regard refers to the specific antibody being highly selective for a particular glycoprotein, exhibiting strong binding and recognition for that glycoprotein, while displaying minimal or no binding to other types of glycoproteins. Specificity may be expressed by determining the binding affinity of the antibody to the glycoprotein, by using biophysical techniques as recognized by a person skilled in the art. The antibodies of the present invention recognize both the sugar moiety and the peptide sequence of the glycoprotein making them very specific.

In one embodiment, the peptide epitope recognized by the VL-domain is part of a specific glycoprotein on the surface of a specific type of cancerous cells.

In addition, the combotope antibodies of the invention is selected to be specific for the combined glycoprotein epitope, i.e. the carbohydrate epitope in combination with the peptide epitope (the combotope), and not for each of the epitopes individually.

In one embodiment, the present invention provides an antibody which binds one or more tumor cells, wherein the one or more tumor cells comprises a carbohydrate epitope associated with a peptide epitope, wherein the antibody is specific against both the carbohydrate epitope and the peptide epitope.

In one embodiment, the specific antibody does not bind specifically to combinations other than the specific combotope, and does not bind (or binds less effectively) the carbohydrate epitope or peptide epitope separately.

In one embodiment, the present invention provides an antibody which binds a tumor cell,
wherein the tumor cell comprises a carbohydrate epitope associated with a peptide epitope,
wherein the antibody is specific for the combination of said carbohydrate epitope and said peptide epitope by virtue of the antibody comprising
   (i) a VH-domain which binds the carbohydrate epitope, and
   (ii) a VL-domain which binds the peptide epitope.

Preferably the carbohydrate epitope is part of a glycoprotein and the peptide epitopes is part of said same glycoprotein. The carbohydrate and peptide epitopes of the tumor are preferably surface displayed in order to facilitate recognition by the antibody.

In one preferred embodiment, the carbohydrate epitope to which the VH-domain binds is selected from mono-Tn, bis-Tn, mono-STn, bis-STn, and/or a combination of monoTn and monoSTn. Hence, in one preferred embodiment, the present invention provides an antibody which binds a tumor cell, wherein the antibody comprises
(i) a VH-domain which binds a monoTn, bisTn, monoSTn, bisSTn, and/or monoTn+monoSTn carbohydrate epitope of a glycoprotein of the tumor cells, and
(ii) a VL-domain which binds a peptide epitope of said plycoprotein of said tumor cells,

wherein the peptide epitope of the glycoprotein of the tumor cell is associated with the carbohydrate epitope of said glycoprotein of the tumor cell, and

wherein the antibody is specific for the combination of said carbohydrate epitope and said peptide epitope of said glycoprotein of said cancer cell.

As disclosed herein, the target peptide epitope of the cell is associated with a carbohydrate epitope. The term “associated with” preferably refers to the peptide epitope and carbohydrate epitope being a continuous epitope, i.e. where the carbohydrate is directly attached to the peptide by virtue of being chemically linked, such as covalently linked. In another embodiment, the peptide epitope and carbohydrate epitope may be a discontinuous epitope, where the “associated with” still refers to the peptide epitope and carbohydrate epitope being in close proximity of one another, but by virtue of the structural configuration of the molecule facilitating this. Hence, a discontinued epitope is where the amino acid hosting the attached carbohydrate epitope is not part of the peptide epitope.

In one embodiment, the present invention provides a tumor cell binding antibody, comprising
(i) a VH-domain which binds a carbohydrate epitope of a glycoprotein of the tumor cell, and
(ii) a VL-domain which binds a peptide epitope of said glycoprotein of said tumor cell,

wherein the antibody is specific for the combination of said carbohydrate epitope and said peptide epitope of said glycoprotein, but not specific for other combinations or one of the epitopes alone.

In one embodiment, the present invention provides a tumor cell binding antibody, comprising
(i) a VH-domain which binds a Tn- and/or STn-carbohydrate epitope of a glycoprotein of the tumor cell, and
(ii) a VL-domain which binds a peptide epitope of said glycoprotein of said tumor cell,

wherein the antibody is specific for the combination of said carbohydrate epitope and said peptide epitope of said glycoprotein, but not specific for other combinations or one of the epitopes alone.

A Tn or a STn epitope may be formed by one or two Tn or STn moieties. In one embodiment, the Tn or STn epitope which interacts with the VH domain is only one Tn or one STn moiety, respectively. In one preferred embodiment, the Tn or STn epitope which interacts with the VH domain is only one Tn or one STn moiety, and said Tn or STn epitope is covalently attached to an amino acid residue, wherein said amino acid residue is part of the peptide epitope which interacts with the VL-domain.

In another embodiment, the Tn or STn epitope is formed by two Tn or STn moieties, respectively, which both interacts with the VH domain. In one preferred embodiment, the Tn or STn epitope which interacts with the VH domain is two Tn or one STn moieties, and said two Tn or STn moieties are covalently attached to two separate amino acid residue, wherein said amino acid residues are part of the peptide epitope which interacts with the VL-domain. In one such embodiment said two separate amino acid residues are adjacent to each other; in another embodiment said two separate amino acid residues are spaced apart by 1, 2, 3 or 4 other amino acid residues.

As disclosed herein, the VL-domain of the combotope antibody binds a peptide epitope of a glycoprotein of a tumor cell. In one embodiment, the peptide epitope is 2, 3, 4, 5, 6, 7, 8, 9, 10, 11, or 12 amino acid residues, preferably 2, 3, or 4 amino acid residues, most preferably 4 amino acid residues. In one embodiment, the peptide epitope which interacts with the VL domain is between 2-4, between 4-6, between 6-8, between 8-10, between 10-12 amino acid residues, such as between 2-12, between 2-10, between 2-8, between preferably 2-6 amino acid residues, most preferably 2-4 amino acid residues.

In a most preferred embodiment, the antibodies of the present invention are different from the prior art antibodies 5E5, 5F7, and 2D9. 5F7 is disclosed in U.S. Ser. No. 11/161,911B2. 5E5 is disclosed in WO2008/040362 and US2021060070A1, and further in the scientific literature Macias-Leon et al 2020; Tarp et al 2007; and Blixt et al 2010. 2D9 is disclosed in the scientific literature Sørensen et al 2006; Tarp et al 2007; and Blixt et al 2010.

In one embodiment, the antibodies of the present invention are different from the prior art antibodies 5E5, 5F7, and 2D9—hence, the antibodies of the present invention do not comprise VL and VH domain combinations as disclosed here:

| | VH domain | VL domain |
|---|---|---|
| 5E5 | SEQ ID NO. 3 | SEQ ID NO. 4 |
| 5F7 | SEQ ID NO. 5 | SEQ ID NO. 6 |
| 2D9 | SEQ ID NO. 7 | SEQ ID NO. 8 |

In one embodiment, the amino acid sequence of the antibodies of the present invention do not comprise an amino acid sequence combination selected from SEQ ID NO. 3+4, SEQ ID NO. 5+6, and SEQ ID NO. 7+8.

Preferably, the antibody of the present invention is a humanized antibody, such as prepared by the method of Clavero-Álvarez et al 2018. “Humanized” forms of non-human antibodies can be chimeric antibodies that contain minimal sequence derived from the non-human antibody. A humanized antibody is generally a human antibody (recipient antibody) in which selected residues are replaced by residues from a non-human antibody (donor antibody). The donor antibody can be any suitable non-human antibody, such as a mouse, rat, rabbit, chicken, or non-human primate antibody having a desired specificity, affinity, or biological effect. For the present invention, the donor antibody is preferably identified by screening an antibody library of the present invention. In some instances, selected framework region residues of the recipient antibody are replaced by the corresponding framework region residues from the donor antibody. Humanized antibodies may also comprise residues that are not found in either the recipient antibody or the donor antibody. In some instances, these modifications are made to further refine antibody performance. The skilled person would be familiar of methods to transform combotope antibodies of the present invention into humanized combotope antibodies.

In a further aspect, the present invention provides nucleic acid sequences encoding the antibodies according to the present invention as disclosed herein.

### I.i Tn-combotopes

As disclosed herein, the VH domain of the combotope antibody binds the carbohydrate epitope, preferably a mono-Tn, bis-Tn, mono-STn or bis-STN carbohydrate epitope.

In one preferred aspect, the VH domain of the combotope antibody binds a Tn-carbohydrate epitope, such a mono-Tn or bis-Tn.

The inventors of the present invention surprisingly made the following discovery: The VH-domain of antibody G2D11 (SEQ ID NO. 1) was by structural characterization in the presence of the bis-Tn-MUC1 peptide APGS*T*AP (where * denotes a GalNAc moiety) found to recognize the two GalNAc moieties, but did not recognize the peptide sequence (see examples 1).

SEQ ID NO. 1 (VH-domain G2D11):
QVQMQQSDAELVKPGASVKISCKASGYIFADHAIHWVKRKPEQGLEWIG
YISPGNDDIKYNEKFKGKATLTADKSSSTAYMQLNSLTSEDSAVYFCKR
SLPGTFDYWGQGTTLTVSS

The novel combotope antibodies disclosed herein are partially based on these structural observations—i.e. that the VH-chain of G2D11 provides recognition support for the glycoside part of the antigen, without being affected/influenced by the peptide part of the antigen. The further development, as disclosed herein, specifies a combotope antibody, where the VL-domain provides specific binding of the peptide epitope of the combotope and the VH-chain provides recognition support for the carbohydrate part of the glycoprotein antigen.

As disclosed in the background section, several anti-Tn antibodies are known in the art, and many of these share the same germline sequences as the G2D11 (at least the essential sequences). But until now it was not know that the VH domain of G2D11 provides exclusive support for the recognition of the Tn carbohydrate epitope, without binding to the peptide epitope. Hence, the prior art anti-Tn antibodies may comprise same germline sequences as the G2D11 responsible for the Tn-binding VH domain, but with no or an unknown binding contribution to the peptide/protein carrier—hence, they are unspecific Tn-binding antibodies, and therefore not combotope antibodies according to the present invention. On the contrary, combotopes of the present invention are screened for by use of the antibody library of the present invention, as further disclosed herein, to obtain antibodies specific for a specific glycoproteins of interest.

In one embodiment, the VH-domain of the combotope antibody of the present invention is a G2D11-like VH-domain. In another embodiment, the amino acids of the VH-domain of the combotope of the present invention resemble the G2D11-like VH-domain in their structural conformation.

G2D11 tolerates binding of any combination TnThr/TnSer, TnSer/TnThr, TnSer/TnSer, TnThr/TnThr. As demonstrated in Example 1 herein, with reference to SEQ ID NO. 1 (VH domain of G2D11), amino acid residues H32, A33, H35, Y50, and S99 are key residues for the binding of the VH domain to one of the GalNAc moiety of bis-Tn-MUC1 peptide, while amino acid residues S52, N55, and D57 are key residues for the binding of the VH domain to the other GalNAc moiety of bis-Tn-MUC1 peptide.

In one embodiment, the VH-domain of the combotope antibody of the present invention comprises an amino acid sequence having at least 60, 65, 70, 75, 80, 85, 90, 92, 94, 96, or 98% sequence homology to SEQ ID NO. 1, preferably at least 80%, more preferably at least 90%, most preferably at least 95% sequence homology to SEQ ID NO. 1; and comprises the amino acid residues H32, A33, H35, Y50, and S99, with respect to SEQ ID NO. 1. Said combotope antibody will provide recognition support for mono-Tn epitopes.

Hence, in one embodiment, combotope antibodies for targeting tumor cells are provided, said antibody comprises a VH and a VL domain; wherein the VH domain of the antibody is a Tn-binding domain and the amino acid sequence of the VH-domain has at least 60, 65, 70, 75, 80, 85, 90, 92, 94, 96, or 98% sequence homology to SEQ ID NO. 1, preferably at least 80%, more preferably at least 90%, most preferably at least 95% sequence homology to SEQ ID NO. 1, wherein the amino acid sequence comprises the amino acid residues H32, A33, H35, Y50, and S99, with respect to SEQ ID NO. 1; and wherein the VL domain of the antibody binds a peptide backbone epitope associated with the Tn-carbohydrate on the tumor cell. The sequence of the VH domain is preferably such that there is no binding to the peptide epitope.

In one embodiment, the VH-domain of the combotope antibody of the present invention comprises an amino acid sequence having at least 60, 65, 70, 75, 80, 85, 90, 92, 94, 96, or 98% sequence homology to SEQ ID NO. 1, preferably at least 80%, more preferably at least 90%, most preferably at least 95% sequence homology to SEQ ID NO. 1; and comprises the amino acid residues S52, N55, and D57, with respect to SEQ ID NO. 1. Said combotope antibody will provide recognition support for mono-Tn epitopes.

Hence, in one embodiment, combopote antibodies for targeting tumor cells are provided, said antibody comprising a VH and a VL domain; wherein the VH domain of the antibody is a Tn-binding domain and the amino acid sequence of the VH-domain has at least 60, 65, 70, 75, 80, 85, 90, 92, 94, 96, or 98% sequence homology to SEQ ID NO. 1, preferably at least 80%, more preferably at least 90%, most preferably at least 95% sequence homology to SEQ ID NO. 1, wherein the amino acid sequence comprises the amino acid residues S52, N55, and D57, with respect to SEQ ID NO. 1; and wherein the VL domain of the antibody binds a peptide backbone epitope associated with the Tn-carbohydrate on the tumor cell. The sequence of the VH domain is preferably such that there is no binding to the peptide epitope.

As demonsted herein, sequence alignment of amino acid sequences of VH-domains of selected Tn-binding mAbs, including the VH-domain of G2D11, showed conserved amino acids in the CDR1, CDR2 and CDR3 regions, related to binding the GalNac (see example 1.2). Specifically, with reference to SEQ ID NO. 1, amino acid residues H32, A33, and H35 in CDR1 should preferably be conserved for the VH domain of the present invention; further, with reference to SEQ ID NO. 1, amino acid residues Y50, S52, N55, and D57 in the CDR2 should preferably be conserved for the VH domain of the present invention; and further, with reference to SEQ ID NO. 1, amino acid residue S99 in the CDR3 should preferably be conserved for the VH domain of the present invention.

In one embodiment, the VH-domain of the combotope antibody comprises an amino acid sequence having at least 60, 65, 70, 75, 80, 85, 90, 92, 94, 96, or 98% sequence homology to SEQ ID NO. 1, preferably at least 80%, more preferably at least 90%, most preferably at least 95% sequence homology to SEQ ID NO. 1; and comprises the amino acid residues H32, A33, H35, Y50, S52, N55, D57, and S99, with respect to SEQ ID NO. 1. Said combotope antibody will provide recognition support for bis-Tn epitopes.

In one embodiment, the amino acid sequence of the VH-domain of the combotope antibody has at least 60, 65, 70, 75, 80, 85, 90, 92, 94, 96, or 98% sequence homology to SEQ ID NO. 1, preferably at least 80%, more preferably at least 90%, most preferably at least 95% sequence homology to SEQ ID NO. 1; and comprises the amino acid residues H32, A33, H35, Y50, S52, N55, D57, and S99, with respect to SEQ ID NO. 1.

In one embodiment, the VH domain of the combotope antibody comprises an amino acid sequence having at least 60, 65, 70, 75, 80, 85, 90, 92, 94, 96, or 98% sequence homology to SEQ ID NO. 1, preferably at least 80%, more preferably at least 90%, most preferably at least 95% sequence homology to SEQ ID NO. 1; and in pairwise alignment with SEQ ID NO. 1, the amino acid sequence of the VH-domain comprises amino acid residues histidine (H), alanine (A), histidine (H), tyrosine (Y), serine (S), asparagine (N), aspartic acid (D), and serine (S) at positions corresponding to amino acid position H32, A33, H35, Y50, S52, N55, D57, and S99 of SEQ ID NO. 1, respectively. The pairwise sequence alignment is performed using scoring matrix: blosum62, gap opening penalty: 10, and gap extension penalty 0.2.

In one embodiment, in pairwise alignment with SEQ ID NO.: 1, the amino acid sequence of the VH-domain of the combotope antibody comprises amino acid residues histidine (H), alanine (A), histidine (H), tyrosine (Y), serine (S), asparagine (N), aspartic acid (D), and serine (S), at positions corresponding to amino acid position H32, A33, H35, Y50, S52, N55, D57, and S99, of SEQ ID NO. 1, respectively; and the amino acid sequence of the VH domain has at least 60, 65, 70, 75, 80, 85, 90, 92, 94, 96, or 98% sequence homology to SEQ ID NO. 1, preferably at least 80%, more preferably at least 90%, most preferably at least 95% sequence homology to SEQ ID NO. 1.

Hence, in one preferred embodiment, antibodies for targeting tumor cells are provided, said antibody comprising a VH and a VL domain; wherein the VH domain of the antibody is a Tn-binding domain and the amino acid sequence of the VH-domain has at least 60, 65, 70, 75, 80, 85, 90, 92, 94, 96, or 98% sequence homology to SEQ ID NO. 1, preferably at least 80%, more preferably at least 90%, most preferably at least 95% sequence homology to SEQ ID NO. 1, wherein the amino acid sequence comprises the amino acid residues H32, A33, H35, Y50, S52, N55, D57, and S99, with respect to SEQ ID NO. 1; and wherein the VL domain of the antibody binds a peptide backbone epitope associated with the Tn-carbohydrate on the tumor cell. The sequence of the VH domain is preferably such that there is no binding to the peptide epitope.

Combotope antibodies of the present invention as described herein, such as the Tn-combotopes disclosed herein, comprise improved binding affinity to a specific antigen epitope termed a combotope comprised of two different epitopes on a glycoprotein. In some embodiments, the antibody comprises a binding affinity (e.g. kD) of between 100 nM to 1 pM, such as less than 100 nM, less than 10 nM, less than 1 nM, less than 100 pM, or even less than 10 pM.

In some embodiment, the combotope antibodies of the present invention are used to treat cancer. In some instances, the cancer is lung, head and neck squamous cell, colorectal, melanoma, liver, classical Hodgkin lymphoma, kidney, gastric, cervical, merkel cell, B-cell lymphoma, or bladder cancer. In a preferred embodiment, the cancer is a solid tumor.

In yet another aspect, provided herein are specific antibodies

In one embodiment, the antibody comprises
(i) a VH-domain having at least 60, 65, 70, 75, 80, 85, 90, 92, 94, 96, or 98% sequence homology to SEQ ID NO. 1, preferably at least 80%, more preferably at least 90%, most preferably at least 95% sequence homology to SEQ ID NO. 1, and comprising the amino acid residues H32, A33, H35, Y50, S52, N55, D57, and S99, with respect to SEQ ID NO. 1, and
(ii) a VL domain having an amino acid sequence selected from of any one of SEQ ID NOs. 9-21.

In one embodiment the present invention provides an antibody comprising a VH domain having the amino acid sequence of SEQ ID NO. 1 and a VL domain having an amino acid sequence selected from any one of SEQ ID NO. 9-21.

In one embodiment, the present invention provides antibodies, wherein the antibody comprises (i) a VH-domain having at least 60, 65, 70, 75, 80, 85, 90, 92, 94, 96, or 98% sequence homology to SEQ ID NO. 1, preferably at least 80%, more preferably at least 90%, most preferably at least 95% sequence homology to SEQ ID NO. 1, and comprising the amino acid residues H32, A33, H35, Y50, S52, N55, D57, and S99, with respect to SEQ ID NO. 1, and (ii) a VL domain having an amino acid sequence selected from of any one of SEQ ID NOs: 9-21; and wherein the antibody is a monoclonal antibody, a polyclonal antibody, a bi-specific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, an isolated complementarity determining region (CDR), a diabody, a fragment comprised of only a single monomeric variable domain, disulfide-linked Fvs (sdFv), an intrabody, an anti-idiotypic (anti-Id) antibody, or ab antigen-binding fragments thereof.

In some embodiment, an antibody comprising (i) a VH domain having an amino acid sequence of SEQ ID NO. 1 and (ii) a VL domain having an amino acid sequence of any one of SEQ ID NOs. 9-21 is used to treat cancer. In some instances, the cancer is lung, head and neck squamous cell, colorectal, melanoma, liver, classical Hodgkin lymphoma, kidney, gastric, cervical, merkel cell, B-cell lymphoma, or bladder cancer. In a preferred embodiment, the cancer is a solid tumor.

Preferably, the selected antibodies of the present invention are a humanized antibody, such as mentioned above.

SEQ ID NO. 9 (VL-domain D3-TnMUC1):
DYKDIQMTQSPSSLAVSVGEKVTMSCKSSQSLLYSSNQKNYLAWYQQKP
GQSPKLLIYWASTRESGVPDRFTGSGSGTDFTLTISSVKAEDLAVYYCQ
QYYSYPLTFGAGTKLEMKR
SEQ ID NO. 10 (VL-domain A3-TnMUC1):
DYKDIVMTQSQKFMSTSVGDRVSITCKASQNVGTAVAWYQQKPGQSPKL
LIYSASNRYTGVPDRFTGSGSGTDFTLTISNVQSEDLADYFCLQHWNYP
LTFGGGTKLEIKR
SEQ ID NO. 11 (VL-domain D2-TnMUC1):
DYKDIQMTQSHKFMSTSVGDRVSITCKASQDVGTAVAWYQQKPGQSPKL
LIYWASTRHTGVPDRFTGSGSGTDFTLTISNVQSEDLADYFCLQHWNYP
LTFGGGTKLEIKR
SEQ ID NO. 12 (VL-domain Ori-TnCD43):
DYKDIQMTQSPASLSASVGETVTITCRASENIYSYLAWYQQKQGKSPQL
LVYNAKTLAEGVPSRFSGSGSGTQFSLKINSLQPEDFGSYYCQHHYGTP
YTFGGGTKLEIKR
SEQ ID NO. 13 (VL-domain H1-TnCD43):
DYKDIVMTQSPSSLAVSVGEKVTMSCKSSQSLLYSSNQKNYLAWYQQKP
GQSPKLLIYWASTRESGVPDRFTGSGSGTDFTLTISSVKAEDLAVYYCQ
QYYSYPWTFGGGTKLEIKR
SEQ ID NO. 14 (VL-domain A1-TnCD43):
DYKDIVMTQSPASLSASVGETVTITCRASENIYSYLAWYQQKQGKSPQL
LVYNAKTLAEGVPSRFSGSGSGTQFSLKINSLQSEDFGSYYCQHHYGTP
YTFGGGTKLEIKR
SEQ ID NO. 15 (VL-domain F4-TnCD43):
DYKDIQMTQSPASLSASVGETVTITCRASENIYSYLAWYQQKQGKSPQL
LVYNAKTLAEGVPSRFSGSGSGTQYSLKINSLQPEDFGSYYCQHFWSTP
YTFGGGTKLEMKR
SEQ ID NO. 16 (VL-domain C5-TnCD43):
DYKDVQMTQSHKFMSTSVGDRVSITCKASQDVSTAVAWYQQKPGQSPKL
LIYWASTRHTGVPDRFTGSGSGTDYTLTISSVQAEDLALYYCQQHYSTP
YTFGGGTKLEIKR
SEQ ID NO. 17 (VL-domain C5-TnCD43):
DYKDIVMTQSHKFMSTSVGDRVSITCKASQDVGTAVAWYQQKPGQSPKL
LIYWASTRHTGVPDRFTGSGSGTDFTLTISNVQSEDLADYFCQQYSSYP
YTFGGGTKLEMKR
SEQ ID NO. 18 (VL-domain D3-TnCD43):
DYKDIVMTQSPSSLAVSAGEKVTMSCKSSQSLLNSRTRKNYLAWYQQKP
GQSPKLLIYWASTRESGVPDRFTGSGSGTDFTLTISNVQSEDLAEYFCQ
QYNSYPLTFGAGTKLEIKR
SEQ ID NO. 19 (VL-domain G3-TnCD43):
DYKDVVMTQSQKFMSTSVRDRVSITCKASQNVGTAVAWYQQKPGQSPKL
LIYSASYRYSGVPDHFTGSGSGTDFTLTISNVQSEDLAEYFCQQYYSYP
YTFGGGTKLEIKR
SEQ ID NO. 20 (VL-domain D7-TnCD43):
DYKDLVLTQSPSSLAVSVGEKVTMSCKSSQSLLYSSNQKNYLAWYQQKP
GQSPKLLIYWASTRESGVPDRFTGSGSGTDFTLTISSVKAEDLAVYYCQ
QYYSYPWTFGGGTKLEMKR
SEQ ID NO. 21 (VL-domain H2-TnCD43):
DYKDIVMTQSPSSLAVSVGEKVTMSCKSSQSLLYSSNQKNYLAWYQQKP
GQSPKLLIYWASTRESGVPDRFTGSGSGTDFTLTISSVKAEDLAVYYCQ
QYYSYPYTFGGGTKLEIKR

### I.ii STn-Combotopes

As disclosed herein, the VH domain of the combotope antibody binds the carbohydrate epitope, preferably a mono-Tn, bis-Tn, mono-STn or bis-STn carbohydrate epitope.

In one preferred aspect, the VH domain of the combotope antibody binds a STn-carbohydrate epitope, such a mono-STn or bis-STn.

The VH-domain of antibody G2D11 (SEQ ID NO. 1) was by structural comparison to the VH domain of 3F1 (SEQ ID NO. 25) modified into a STn-binding VH-domain (SEQ ID NO. 28) (see example 5). Compared to G2D11 (SEQ ID NO. 1), the STn binding VH domain (SEQ ID NO. 28) has the following the following amino acid residue changes: I28T, A30T, P101L, de/G102, T103A and F104L.

SEQ ID NO. 28 (VH domain G2D11 mutant M2: LAL-TFT)
QSDAELVKPGASVKISCKASGYTFTDHAIHWVKRKPEQGLEWIGYISPG
NDDIKYNEKFKGKATLTADKSSSTAYMQLNSLTSEDSAVYFCKRSLLAL
DYWGQGTTLTVSS

The novel STn-combotope antibodies disclosed herein are based on these structural modification and further development, as disclosed herein, and provides specific combotope recognition, wherein the VH-chain provides recognition support for the carbohydrate part of the combotope on the glycoprotein antigen, while the VL-domain provides recognition support for the peptide part of the combotope on the glycoprotein antigen.

In one embodiment, the VH-domain of the combotope antibody of the present invention is a SEQ ID NO. 28-like VH-domain. In one embodiment, the amino acids of the VH-domain of the combotope of the present invention resemble the SEQ ID NO. 28-like VH-domain in their structural conformation.

As demonstrated in Example 5, amino acid residues T28, T30, L101, A102, and L103 with respect to SEQ ID NO. 28, are key amino acids for the STn-specificity. They are need for accommodation of the sialyl—i.e. for making extra space for sialyl for STn binding.

In one embodiment, the VH-domain of the combotope antibody comprises an amino acid sequence having at least 60, 65, 70, 75, 80, 85, 90, 92, 94, 96, or 98% sequence homology to SEQ ID NO. 28, preferably at least 80%, more preferably at least 90%, most preferably at least 95% sequence homology to SEQ ID NO. 28; and comprises the amino acid residues T28, T30, H32, A33, H35, Y50, S99, L101, A102 and L103, with respect to SEQ ID NO. 28. Said combotope antibody will provide recognition support for mono-STn epitopes.

Hence, in one embodiment, antibodies for targeting tumor cells are provided, said antibody comprising a VH and VL domain; wherein the VH domain of the antibody is a STn-binding domain and the amino acid sequence of the VH-domain has at least 60, 65, 70, 75, 80, 85, 90, 92, 94, 96, or 98% sequence homology to SEQ ID NO. 28, preferably at least 80%, more preferably at least 90%, most preferably at least 95% sequence homology to SEQ ID NO. 28; wherein the amino acid sequence comprises the amino acid residues T28, T30, H32, A33, H35, Y50, S99, L101, A102 and L103, with respect to SEQ ID NO. 28; and wherein the VL domain of the antibody binds a peptide backbone associated with the STn-carbohydrate on the tumor cell. The sequence of the VH domain is preferably such that there is no binding to the peptide epitope.

In one embodiment, the VH-domain of the combotope antibody comprises an amino acid sequence having at least 60, 65, 70, 75, 80, 85, 90, 92, 94, 96, or 98% sequence homology to SEQ ID NO. 28, preferably at least 80%, more preferably at least 90%, most preferably at least 95% sequence homology to SEQ ID NO. 28; and comprises the amino acid residues T28, T30, S52, N55, D57, L101, A102 and L103, with respect to SEQ ID NO. 28. Said combotope antibody will provide recognition support for mono-STn epitopes.

Hence, in one embodiment, antibodies for targeting tumor cells are provided, said antibody comprising a VH and VL domain; wherein the VH domain of the antibody is a STn-binding domain and the amino acid sequence of the VH-domain has at least 60, 65, 70, 75, 80, 85, 90, 92, 94, 96, or 98% sequence homology to SEQ ID NO. 28, preferably at least 80%, more preferably at least 90%, most preferably at least 95% sequence homology to SEQ ID NO. 28; wherein the amino acid sequence comprises the amino acid residues T28, T30, S52, N55, D57, L101, A102 and L103, with respect to SEQ ID NO. 28; and wherein the VL domain of the antibody binds a peptide backbone associated with the STn-carbohydrate on the tumor cell. The sequence of the VH domain is preferably such that there is no binding to the peptide epitope.

In one embodiment, the VH-domain of the combotope antibody comprises an amino acid sequence having at least 60, 65, 70, 75, 80, 85, 90, 92, 94, 96, or 98% sequence homology to SEQ ID NO. 28, preferably at least 80%, more preferably at least 90%, most preferably at least 95% sequence homology to SEQ ID NO. 28; and comprises the amino acid residues T28, T30, H32, A33, H35, Y50, S52, N55, D57, S99, L101, A102 and L103, with respect to SEQ ID NO. 28. Said combotope antibody will provide recognition support for bis-STn epitopes.

In one embodiment, the amino acid sequence of the VH-domain of the combotope antibody has at least 60, 65, 70, 75, 80, 85, 90, 92, 94, 96, or 98% sequence homology to SEQ ID NO. 28, preferably at least 80%, more preferably at least 90%, most preferably at least 95% sequence homology to SEQ ID NO. 28; and comprises the amino acid residues T28, T30, H32, A33, H35, Y50, S52, N55, D57, S99, L101, A102 and L103, with respect to SEQ ID NO. 28.

In one embodiment, the VH domain of the combotope antibody comprises an amino acid sequence having at least 60, 65, 70, 75, 80, 85, 90, 92, 94, 96, or 98% sequence homology to SEQ ID NO. 28, preferably at least 80%, more preferably at least 90%, most preferably at least 95% sequence homology to SEQ ID NO. 28; and in pairwise alignment with SEQ ID NO. 28 the amino acid sequence of the VH-domain comprises amino acid residues threonine (T), threonine (T), histidine (H), alanine (A), histidine (H), tyrosine (Y), serine (S), asparagine (N), aspartic acid (D), serine (S), leucine (L), alanine (A), and leucine (L) at positions corresponding to amino acid position T28, T30, H32, A33, H35, Y50, S52, N55, D57, S99, L101, A102 and L103 of SEQ ID NO. 28, respectively. The pairwise sequence alignment is performed using scoring matrix: blosum62, gap opening penalty: 10, and gap extension penalty 0.2.

In one embodiment, in pairwise alignment with SEQ ID NO. 28, the amino acid sequence of the VH-domain of the combotope antibody comprises amino acid residues threonine (T), threonine (T), histidine (H), alanine (A), histidine (H), tyrosine (Y), serine (S), asparagine (N), aspartic acid (D), serine (S), leucine (L), alanine (A), and leucine (L), at positions corresponding to amino acid position T28, T30, H32, A33, H35, Y50, S52, N55, D57, S99, L101, A102 and L103, of SEQ ID NO. 28, respectively; and the amino acid sequence of the VH domain has at least 60, 65, 70, 75, 80, 85, 90, 92, 94, 96, or 98% sequence homology to SEQ ID NO. 28, preferably at least 80%, more preferably at least 90%, most preferably at least 95% sequence homology to SEQ ID NO. 28.

Hence, in one embodiment, antibodies for targeting tumor cells are provided, said antibody comprising a VH and VL domain; wherein the VH domain of the antibody is a STn-binding domain and the amino acid sequence of the VH-domain has at least 60, 65, 70, 75, 80, 85, 90, 92, 94, 96, or 98% sequence homology to SEQ ID NO. 28, preferably at least 80%, more preferably at least 90%, most preferably at least 95% sequence homology to SEQ ID NO. 28; wherein the amino acid sequence comprises the amino acid residues T28, T30, H32, A33, H35, Y50, S52, N55, D57, S99, L101, A102 and L103, with respect to SEQ ID NO. 28; and wherein the VL domain of the antibody binds a peptide backbone associated with the STn-carbohydrate on the tumor cell. The sequence of the VH domain is preferably such that there is no binding to the peptide epitope.

Combotope antibodies of the present invention as described herein, such as the STn-combotopes disclosed herein, comprise improved binding affinity to a specific antigen epitope, the combotope (compared to other epitopes on healthy or cancer cells). In some embodiment, the antibody comprises a binding affinity (e.g., kD) of between 100 nM to 1 pM, such as less than 100 nM, less than 10 nM, less than 1 nM, less than 100 pM, or even less than 10 pM.

In some embodiment, the antibodies of the present invention are used to treat cancer. In some instances, the cancer is lung, head and neck squamous cell, colorectal, melanoma, liver, classical Hodgkin lymphoma, kidney, gastric, cervical, merkel cell, B-cell lymphoma, or bladder cancer. In a preferred embodiment, the cancer is a solid tumor.

In yet another aspect, the present invention discloses specific antibodies.

In one embodiment, the combotope antibody of the present invention comprises
(i) a VH-domain having at least 60, 65, 70, 75, 80, 85, 90, 92, 94, 96, or 98% sequence homology to SEQ ID NO. 28, preferably at least 80%, more preferably at least 90%, most preferably at least 95% sequence homology to SEQ ID NO. 28, and comprising the amino acid residues T28, T30, H32, A33, H35, Y50, S52, N55, D57, S99, L101, A102 and L103, with respect to SEQ ID NO. 28, and
(ii) a VL domain having an amino acid sequence selected from of any one of SEQ ID NOs. 22-24.

In one embodiment the present invention provides an antibody comprising a VH domain having the amino acid sequence of SEQ ID NO. 28 and a VL domain having an amino acid sequence selected from any one of SEQ ID NO. 22-24.

In one embodiment, the present invention provides antibodies, wherein the antibody comprises (i) a VH-domain having at least 60, 65, 70, 75, 80, 85, 90, 92, 94, 96, or 98% sequence homology to SEQ ID NO. 28, preferably at least 80%, more preferably at least 90%, most preferably at least 95% sequence homology to SEQ ID NO. 28, and comprising the amino acid residues T28, T30, H32, A33, H35, Y50, S52, N55, D57, S99, L101, A102 and L103, with respect to SEQ ID NO. 28, and (ii) a VL domain having an amino acid sequence selected from of any one of SEQ ID NOs. 22-24; and wherein the antibody is a monoclonal antibody, a polyclonal antibody, a bi-specific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, an isolated complementarity determining region (CDR), a diabody, a fragment comprised of only a single monomeric variable domain, disulfide-linked Fvs (sdFv), an intrabody, an anti-idiotypic (anti-Id) antibody, or ab antigen-binding fragments thereof.

In some embodiment, an antibody comprising (i) a VH domain having an amino acid sequence of SEQ ID NO. 28 and (ii) a VL domain having an amino acid sequence of any one of SEQ ID NOs. 22-24 is used to treat cancer. In some instances, the cancer is lung, head and neck squamous cell, colorectal, melanoma, liver, classical Hodgkin lymphoma, kidney, gastric, cervical, merkel cell, B-cell lymphoma, or bladder cancer. In a preferred embodiment, the cancer is a solid tumor.

Preferably, the selected antibody of the present invention is a humanized antibody, such as mentioned above.

SEQ ID NO. 22 (VL-domain C4-STnMUC1):
IVMTQSPSSLAVSAGEKVTMSCKSSQSLLNSRTRKNYLAWYQQKPGQSP
KLLIYWASTRHTGVPDRFTGSGSGTDFTLTISNVQSEDLAEYFCQQYNS
YPYTFGGGTKLEIKR
SEQ ID NO. 23 (VL-domain D3-STnMUC1):
IVMTQSPSSLAVSAGEKVTMSCKSSQSLLNSRTRKNYLAWYQQKPGQSP
KLLIYWASTRHTGVPDRFTGSGSGTDFTLTISNVQSEDLAEYFCQQYNS
YPYTFGGGTKLEIKR
SEQ ID NO. 24 (VL-domain C7-STnMUC1):
VVVTQTPLSLPVSLGDQASISCRSSQSLVHSNGNTYLHWYLQKPGQSPK
LLIYKVSNRFSGVPDRFSGSGSGTDFTLKISRVEAEDLGVYFCSQSTHV
PRTFGGGTKLEIKR

### II. Antibody Library

In one aspect, the present invention provides an antibody library for in-vitro identification of a specific antibody which binds glycoproteins, such as glycoproteins on tumor cells. Hence, in one embodiment, the present invention provides an antibody library for in-vitro identification of a specific antibody which binds one or more tumor cells.

As disclosed herein, tumor cells often comprise Tn and/or STn glycosylation epitopes on specific glycoproteins on the surface of the cancer cells. The antibody library of the present invention facilitates identification of antibodies with improved specificity towards tumor cells by virtue of the antibodies being specific both towards the carbohydrate epitope (Tn and/or STn) as well as towards the peptide epitope in the protein backbones associated with the carbohydrate (epitope). Specifically, such improved antibodies comprise a VH domain which efficiently binds the carbohydrate epitope of the glycoprotein, and a VL domain which efficiently binds the peptide epitope of the glycoprotein associated with the carbohydrate epitope, and the antibody is thereby specific for the combination of said carbohydrate epitope and said peptide epitope of said glycoprotein, the combined epitopes being termed a “combotope”.

As mentioned previously, the VH-domain of antibody G2D11 (SEQ ID NO. 1) was by structural characterization found to particularly recognize the carbohydrate epitope of the glycoprotein epitope bis-Tn-MUC1, while it did not recognize the peptide sequence of said glycoprotein epitope (see examples 1).

Based on these structural observations, the antibody library of the present invention was conceptualized, wherein each antibody of the library comprises a specific preselected VH chain which provides recognition support for the carbohydrate part of the combotope antigen, while the VL-domain is variable, including one or more VL domains being specific for a particular peptide sequence and thus a particular glycoprotein, creating a library which can be screened for specific combotope antibodies, of which the VL-domain will provide recognition support to the peptide epitope within the combotope of said glycoprotein.

Based on the binding and structural data presented herein (i.e. key amino acids for Tn and STn binding, in combination with the specific G2D11 versatile VH sequence), the antibody library of the present invention can be used to identify specific glycoprotein combotope antibodies, where the VL-domain is specific for the peptide epitope of choice.

In one aspect, the invention provides an antibody library, wherein each of the antibodies in the library comprises two antibody domains: (i) a first antibody domain which binds a carbohydrate epitope of a glycoprotein, such as a glycoprotein of the tumor cell, and (ii) a second antibody domain selected from a repertoire of antibody domains, wherein the repertoire of antibody domains comprises one or more domains which binds a peptide epitope of said glycoprotein; wherein said library is for in-vitro identification of a specific antibody from said library for targeting tumor cells, and wherein the specific antibody is specific for the combination of said carbohydrate epitope and said peptide epitope of said glycoprotein—i.e. the specific antibody is a combotope antibody.

In one aspect, the invention provides an antibody library, wherein each of the antibodies in the library comprises two antibody domains: (i) a first antibody domain which binds a carbohydrate epitope of a glycoprotein of the tumor cell, and (ii) a second antibody domain selected from a repertoire of antibody domains, wherein the repertoire of antibody domains comprises one or more domains which binds a peptide epitope of said glycoprotein of said tumor cell; wherein said library is for in-vitro identification of a specific antibody from said library for targeting tumor cells, and wherein the specific antibody is specific for the combination of said carbohydrate epitope and said peptide epitope of said glycoprotein—i.e. the specific antibody is a combotope antibody.

In one embodiment, the first antibody domain is a VH domain and the second antibody domain is a VL domain. In another embodiment, both the first and second antibody are VH domains, but different from one another.

In one aspect, the present invention provides an antibody library, wherein each of the antibodies in the antibody library comprises
(i) a VH-domain which binds a carbohydrate epitope on a glycoprotein of the tumor cell, and
(ii) a VL-domain selected from a repertoire of VL-domains, wherein the repertoire of VL-domains comprises one or more VL-domains which binds a peptide epitope on said glycoprotein of the tumor cell,

for in-vitro identification of a specific antibody from said library which binds a tumor cell, wherein the specific antibody is specific for the combination of said carbohydrate epitope and said peptide epitope of said glycoprotein, the combined epitope being termed a “combotope”.

In one embodiment, the present invention provides an antibody library for in-vitro identification of a specific antibody which binds a tumor cell,
wherein each of the antibodies in the antibody library comprises (ii) a VL-domain selected from a repertoire of VL-domains, wherein the repertoire of VL-domains comprises one or more VL-domains which binds a peptide epitope on a glycoprotein of a tumor cell, and (i) a VH-domain which binds a carbohydrate epitope on said glycoprotein of said tumor cell and which does not contribute or interfere with binding said peptide epitope of said glycoprotein of said tumer cell,
wherein the specific antibody is specific for the combination of said carbohydrate epitope and said peptide epitope of said glycoprotein, the combined epitope being termed a “combotope” i.e. the specific antibody is a combotope antibody.

The VH chain of each antibody encoded by the library thereby pre-selects for a desired glycoform specificity, while the VL chain is selected from a repertoire of LV domains and will determine the peptide backbone specificity and thereby the glycoprotein specificity.

In one embodiment, the VH chain of each antibody encoded by the library pre-selects for a desired glycoform specificity, without interfering with the peptide backbone specificity, while the VL chain is selected from a repertoire of LV domains and will determine the peptide backbone specificity and thereby the glycoprotein specificity. The structural studies disclosed herein (Example 1) provide clear evidence that the VH domain of G2D11 has no interaction with the peptide/protein carrier, and that peptide/protein interaction of the antibodies entirely comes from contribution via the VL-chain, as exemplified with antibodies obtain from the library of the present invention (e.g. antibodies Tn-MUUC1 and Tn-CD43; see Examples 3 and 4).

In one preferred embodiment, the the peptide epitope and carbohydrate epitope are a common continuous epitope, i.e. where the carbohydrate is in close proximity to the peptide by virtue of being chemically linked, such as by a covalent bond. In another embodiment, the peptide epitope and carbohydrate epitope may be a common discontinuous epitope, where the “associated with” still refers to the peptide epitope and carbohydrate epitope being in close proximity of one another, but by virtue of the structural configuration of the molecule facilitating the common epitope.

The antibodies herein are selected from a monoclonal antibody, a polyclonal antibody, a bi-specific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, an isolated complementarity determining region (CDR), a diabody, a fragment comprised of only a single monomeric variable domain, disulfide-linked Fvs (sdFv), an intrabody, an antiidiotypic (anti-Id) antibody, and ab antigen-binding fragments thereof.

In one preferred embodiment, the antibodies encoded by the antibody library of the invention are scFv, wherein the VH-domain is linked to the VL-domain.

In one preferred embodiment, the libraries disclosed herein comprise scFv antibodies, comprising a VH domain and a VL domain, where both domains are present in a single polypeptide chain. In some embodiments, the Fv polypeptide further comprises a polypeptide linker between the VH and VL domains allowing the scFv to form the desired structure for antigen binding. In one embodiment, the linker is selected from (GGGGS)ₙ where in is 1, 2, 3, 4, 5, or 6.

In one embodiment, the VH domain of each antibody in the antibody library specifically binds one or more carbohydrate epitope selected from Tn and/or STn. In one embodiment, the VH domain of each antibody encoded by the antibody library specifically binds one or more Tn moieties, such as a mono-Tn epitope or a bis-Tn epitope. In another embodiment, the VH domain of each antibody encoded by the antibody library specifically binds one or more STn-moieties, such as a mono-STn epitope or a bis-STn epitope. In yet another embodiment, the VH domain of some of the antibodies encoded by the antibody library specifically bind one or more Tn-moieties, while the VH domain of other antibodies encoded by the antibody library specifically bind one or more STn-moieties.

As disclosed above, the VH domain is specified for each antibody in the antibody library to specifically bind a specific carbohydrate epitope being characteristic of cancer cells. On the contrary, the VL domains of the antibodies in the antibody library vary from one antibody to the other, representing a repertoire of VL domains recognizing different peptides from the glycoprotein in question, such that a repertoire of VL-domains is generated, to be screened with the intent of identifying combotope antibodies which have specificity towards glycoproteins on tumor cells by virtue of the VH domain binding a carbohydrate epitope of the glycoprotein on the tumor cell and the VL domain binding a peptide epitope on said glycoprotein on the tumor cell.

In one embodiment, the repertoire of VL-domains is generated from a naïve immune repertoire, an immunized immune repertoire in a suitable animal, or a synthetically produced repertoire.

In one embodiment, the repertoire of VL-domains encoded by the antibody library is a naïve immune repertoire from an animal, such as a mouse or human. Othe suitable animals are pigs, rats, dogs, horses, rabbits known to the skilled artisan.

In one preferred embodiment, the antibody library is phage display library.

### II.i Tn-Template Antibody Library

Provided herein are Tn-template antibody libraries, wherein the first domain of each antibody in the library is a Tn-binding domain, while the second domain is selected from a repertoire of antibody domains comprising one or more domain binding a peptide epitope on a glycoprotein of interest associated with the Tn epitope, as disclosed above.

Provided herein are Tn-template antibody libraries, wherein the VH domain of each antibody in the library is a Tn-binding VH domain, while the VL domain is selected from a repertoire of VL domains comprising one or more VL-domains binding a peptide epitope on a glycoprotein of interest associated with the Tn epitope, as disclosed above.

In one embodiment, the present invention provides mono-Tn-template antibody libraries, wherein the VH domain of each antibody in the library is a mono-Tn-binding VH domain, while the VL domain is selected from a repertoire of VL domains comprising one or more VL-domains binding a peptide epitope on a glycoprotein of interest associated with the Tn epitope (supra).

In one embodiment, the present invention provides bis-Tn-template antibody libraries, wherein the VH domain of each antibody in the library is a bis-Tn-binding VH domain, while the VL domain is selected from a repertoire of VL domains comprising one or more VL-domains binding a peptide epitope on a glycoprotein of interest associated with the Tn epitope (supra).

In preferred embodiment, the present invention provides antibody libraries which may be used to select for antibodies which binds mono-Tn and bis-Tn epitopes, wherein the VH domain of each antibody in the library is a mono- as well as bis-Tn-binding VH domain, while the VL domain is selected from a repertoire of VL domains comprising one or more VL-domains binding a peptide epitope on glycoprotein of interest associated with the Tn epitope (supra).

In one embodiment, the VH-domain of each antibody in the Tn-template antibody library is a G2D11-like VH-domain (SEQ ID NO. 1). In one embodiment, the amino acids of the VH-domain of each antibody in the library resemble the G2D11-like VH-domain in their structural conformation.

In one embodiment, the VH-domain of the Tn-template antibody library comprises an amino acid sequence having at least 60, 65, 70, 75, 80, 85, 90, 92, 94, 96, or 98% sequence homology to SEQ ID NO. 1, preferably at least 80%, more preferably at least 90%, most preferably at least 95% sequence homology to SEQ ID NO. 1; and comprises amino acid residues H32, A33, H35, Y50, and S99, with respect to SEQ ID NO. 1.

Hence, in one embodiment, an antibody library is provided for selecting tumor-targeting antibodies, wherein each antibody of the antibody library comprises a VH and a VL domain; wherein the VH domain of each antibody is a Tn-binding VH domain and the VH-domain comprises an amino acid sequence having at least 60, 65, 70, 75, 80, 85, 90, 92, 94, 96, or 98% sequence homology to SEQ ID NO. 1, preferably at least 80%, more preferably at least 90%, most preferably at least 95% sequence homology to SEQ ID NO. 1, and comprises amino acid residues H32, A33, H35, Y50, and S99, with respect to SEQ ID NO. 1; and wherein the VL domain is selected from a repertoire of VL domains, wherein the repertoire comprises at least one VL domain which binds a peptide backbone epitope associated with the Tn-carbohydrate on the tumor cell. The sequence of the VH domain is preferably such that there is no binding to the peptide epitope.

In one embodiment, the VH-domain of the Tn-template antibody library comprises an amino acid sequence having at least 60, 65, 70, 75, 80, 85, 90, 92, 94, 96, or 98% sequence homology to SEQ ID NO. 1, preferably at least 80%, more preferably at least 90%, most preferably at least 95% sequence homology to SEQ ID NO. 1; and comprises amino acid residues S52, N55, and D57, with respect to SEQ ID NO. 1.

Hence, in one embodiment, an antibody library is provided for selecting tumor-targeting antibodies, wherein each antibody of the antibody library comprises a VH and a VL domain; wherein the VH domain of each antibody is a Tn-binding VH domain and the VH-domain comprises an amino acid sequence having at least 60, 65, 70, 75, 80, 85, 90, 92, 94, 96, or 98% sequence homology to SEQ ID NO. 1, preferably at least 80%, more preferably at least 90%, most preferably at least 95% sequence homology to SEQ ID NO. 1, and comprises amino acid residues S52, N55, and D57, with respect to SEQ ID NO. 1; and wherein the VL domain is selected from a repertoire of VL domains, wherein the repertoire comprises at least one VL domain which binds a peptide backbone epitope associated with the Tn-carbohydrate on the tumor cell. The sequence of the VH domain is preferably such that there is no binding to the peptide epitope.

In one embodiment, the VH-domain of the Tn-template antibody library comprises an amino acid sequence having at least 60, 65, 70, 75, 80, 85, 90, 92, 94, 96, or 98% sequence homology to SEQ ID NO. 1, preferably at least 80%, more preferably at least 90%, most preferably at least 95% sequence homology to SEQ ID NO. 1; and comprises amino acid residues H32, A33, H35, Y50, S52, N55, D57, and S99, with respect to SEQ ID NO. 1.

Hence, in one preferred embodiment, an antibody library is provided for selecting tumor-targeting antibodies, wherein each antibody of the antibody library comprises a VH and a VL domain; wherein the VH domain of each antibody is a Tn-binding VH domain and the VH-domain comprises an amino acid sequence having at least 60, 65, 70, 75, 80, 85, 90, 92, 94, 96, or 98% sequence homology to SEQ ID NO. 1, preferably at least 80%, more preferably at least 90%, most preferably at least 95% sequence homology to SEQ ID NO. 1, and comprises amino acid residues H32, A33, H35, Y50, S52, N55, D57, and S99, with respect to SEQ ID NO. 1; and wherein the VL domain is selected from a repertoire of VL domains, wherein the repertoire comprises at least one VL domain which binds a peptide backbone epitope associated with the Tn-carbohydrate on the tumor cell. The sequence of the VH domain is preferably such that there is no binding to the peptide epitope.

In one embodiment, the VH domain of the Tn-template antibody library comprises an amino acid sequence having at least 60, 65, 70, 75, 80, 85, 90, 92, 94, 96, or 98% sequence homology to SEQ ID NO. 1, preferably at least 80%, more preferably at least 90%, most preferably at least 95% sequence homology to SEQ ID NO. 1; and in pairwise alignment with SEQ ID NO.: 1, the amino acid sequence of the VH-domain comprises amino acid residues histidine (H), alanine (A), histidine (H), tyrosine (Y), serine (S), asparagine (N), aspartic acid (D), and serine (S) at positions corresponding to amino acid position H32, A33, H35, Y50, S52, N55, D57, and S99 of SEQ ID NO. 1, respectively.

The pairwise sequence alignment is performed using scoring matrix: blosum62, gap opening penalty: 10, and gap extension penalty 0.2.

Hence, in one embodiment, an antibody library is provided for selecting tumor-targeting antibodies, wherein each antibody of the antibody library comprises a VH and VL domain; wherein the VH domain of each antibody is a Tn-binding VH domain and the amino acid sequence of the VH-domain in pairwise alignment with SEQ ID NO. 1 comprises amino acid residues histidine (H), alanine (A), histidine (H), tyrosine (Y), serine (S), asparagine (N), aspartic acid (D), and serine (S) at positions corresponding to amino acid position H32, A33, H35, Y50, S52, N55, D57, and S99 of SEQ ID NO. 1, respectively; and the amino acid sequence of the VH domain has at least 60, 65, 70, 75, 80, 85, 90, 92, 94, 96, or 98% sequence homology to SEQ ID NO. 1, preferably at least 80%, more preferably at least 90%, most preferably at least 95% sequence homology to SEQ ID NO. 1.

In one embodiment the first antibody domain is a mono- or bis-Tn-binding VH domain, and wherein said VH-domain comprises an amino acid sequence having at least 90% sequence homology to SEQ ID NO. 1 and comprising amino acid residues H32, A33, H35, Y50, and S99 and/or amino acid residues S52, N55, and D57, with respect to SEQ ID NO. 1, preferably comprising amino acid residues H32, A33, H35, Y50, and S99 and amino acid residues S52, N55, and D57, with respect to SEQ ID NO. 1.

The Tn-template library is useful for screening for antibodies which bind combotopes comprising a Tn epitope.

### II.ii STn-Template Antibody Library

Provided herein are STn-template antibody libraries, wherein the first domain of each antibody in the library is a STn-binding domain, while the second domain is selected from a repertoire of antibody domains comprising one or more domain binding a peptide epitope on a glycoprotein of interest associated with the STn epitope, as disclosed above.

Provided herein are STn-template antibody libraries, wherein the VH domain of each antibody in the library is a STn-binding VH domain, while the VL domain is selected from a repertoire of VL domains comprising one or more VL-domains binding a peptide epitope on a glycoprotein of interest associated with the STn epitope, as describes above.

In one embodiment, the present invention provides mono-STn-template antibody libraries, wherein the VH domain of each antibody in the library is a mono-STn-binding VH domain, while the VL domain is selected from a repertoire of VL domains comprising one or more VL-domains binding a peptide epitope on a glycoprotein of interest associated with the STn epitope (supra).

In one embodiment, the present invention provides bis-STn-template antibody libraries, wherein the VH domain of each antibody in the library is a bis-STn-binding VH domain, while the VL domain is selected from a repertoire of VL domains comprising one or more VL-domains binding a peptide epitope on a glycoprotein of interest associated with the STn epitope (supra).

In a preferred embodiment, the present invention provides antibody libraries which may be used to select for antibodies which binds mono-STn and bis-STn epitopes, wherein the VH domain of each antibody in the library is a mono- as well as bis-STn-binding VH domain, while the VL domain is selected from a repertoire of VL domains comprising one or more VL-domains binding a peptide epitope on a glycoprotein of interest associated with the STn epitope (supra).

As disclosed herein, the VH-domain of antibody 3F1 (SEQ ID NO. 25) efficiently binds STn. In one embodiment, the VH-domain of each antibody in the STn-template antibody library is a 3F1-like VH-domain (SEQ ID NO. 25). In one embodiment, the amino acids of the VH-domain of each antibody in the library resemble the 3F1-like VH-domain in their structural conformation.

A potential disadvantage of 3F1 is that is does not express well, however, G2D11 is very stable and easy to produce compared to 3F1. For example, G2D11 is very well expressed in Pichia pastoris, while 3F1 does not express so well. In addition, we wanted to learn the molecular basis of how to convert an anti-Tn to an anti-STn.

By sequence comparison of G2D11 and 3F1, amino acid residues were identified which might affect Tn vs STn specificity. A modified G2D11 VH domain was prepared, to simulate the 3F1 VH domain, for obtaining STn specificity (see example 5). This altered G2D11 VH domain is provided herein as SEQ ID NO. 28. Compared to G2D11 (SEQ ID NO. 1), the altered STn binding VH domain has the following amino acid residue changes: I28T, A30T, P101L, de/G102, T103A and F104L. In one embodiment, the VH-domain of each antibody in the STn-template antibody library is a SEQ ID NO. 28-like VH-domain. In one embodiment, the amino acids of the VH-domain of each antibody in the library resemble SEQ ID NO. 28 in their structural conformation.

In one embodiment, the VH-domain of the STn-template antibody library comprises an amino acid sequence having at least 60, 65, 70, 75, 80, 85, 90, 92, 94, 96, or 98% sequence homology to SEQ ID NO. 28, preferably at least 80%, more preferably at least 90%, most preferably at least 95% sequence homology to SEQ ID NO. 28, and comprises amino acid residues T28, T30, H32, A33, H35, Y50, S99, L101, A102 and L103, with respect to SEQ ID NO. 28.

Hence, in one embodiment, an antibody library is provided for selecting tumor-targeting antibodies, wherein each antibody of the antibody library comprises a VH and a VL domain; wherein the VH domain of each antibody is a STn-binding VH domain and the VH-domain comprises an amino acid sequence having at least 60, 65, 70, 75, 80, 85, 90, 92, 94, 96, or 98% sequence homology to SEQ ID NO. 28, preferably at least 80%, more preferably at least 90%, most preferably at least 95% sequence homology to SEQ ID NO. 28, and comprising amino acid residues T28, T30, H32, A33, H35, Y50, S99, L101, A102 and L103, with respect to SEQ ID NO. 28; and wherein the VL domain is selected from a repertoire of VL domains, wherein the repertoire comprises at least one VL domain which binds a peptide backbone epitope associated with the STn-carbohydrate on the tumor cell.

In one embodiment, the VH-domain of the STn-template antibody library comprises an amino acid sequence having at least 60, 65, 70, 75, 80, 85, 90, 92, 94, 96, or 98% sequence homology to SEQ ID NO. 28, preferably at least 80%, more preferably at least 90%, most preferably at least 95% sequence homology to SEQ ID NO. 28, and comprises amino acid residues T28, T30, S52, N55, D57, L101, A102 and L103, with respect to SEQ ID NO. 28.

Hence, in one embodiment, an antibody library is provided for selecting tumor-targeting antibodies, wherein each antibody of the antibody library comprises a VH and a VL domain; wherein the VH domain of each antibody is a STn-binding VH domain and the VH-domain comprises an amino acid sequence having at least 60, 65, 70, 75, 80, 85, 90, 92, 94, 96, or 98% sequence homology to SEQ ID NO. 28, preferably at least 80%, more preferably at least 90%, most preferably at least 95% sequence homology to SEQ ID NO. 28, and comprising amino acid residues T28, T30, S52, N55, D57, L101, A102 and L103, with respect to SEQ ID NO. 28; and wherein the VL domain is selected from a repertoire of VL domains, wherein the repertoire comprises at least one VL domain which binds a peptide backbone epitope associated with the STn-carbohydrate on the tumor cell.

In one embodiment, the VH-domain of the STn-template antibody library comprises an amino acid sequence having at least 60, 65, 70, 75, 80, 85, 90, 92, 94, 96, or 98% sequence homology to SEQ ID NO. 28, preferably at least 80%, more preferably at least 90%, most preferably at least 95% sequence homology to SEQ ID NO. 28, and comprises amino acid residues T28, T30, H32, A33, H35, Y50, S52, N55, D57, S99, L101, A102 and L103, with respect to SEQ ID NO. 28.

Hence, in one embodiment, an antibody library is provided for selecting tumor-targeting antibodies, wherein each antibody of the antibody library comprises a VH and a VL domain; wherein the VH domain of each antibody is a STn-binding VH domain and the VH-domain comprises an amino acid sequence having at least 60, 65, 70, 75, 80, 85, 90, 92, 94, 96, or 98% sequence homology to SEQ ID NO. 28, preferably at least 80%, more preferably at least 90%, most preferably at least 95% sequence homology to SEQ ID NO. 28, and comprising amino acid residues T28, T30, H32, A33, H35, Y50, S52, N55, D57, S99, L101, A102 and L103, with respect to SEQ ID NO. 28; and wherein the VL domain is selected from a repertoire of VL domains, wherein the repertoire comprises at least one VL domain which binds a peptide backbone epitope associated with the STn-carbohydrate on the tumor cell.

In one embodiment, the VH domain of the STn-template antibody library comprises an amino acid sequence having at least 60, 65, 70, 75, 80, 85, 90, 92, 94, 96, or 98% sequence homology to SEQ ID NO. 28, preferably at least 80%, more preferably at least 90%, most preferably at least 95% sequence homology to SEQ ID NO. 28, and in pairwise alignment with SEQ ID NO. 28, the amino acid sequence of the VH-domain comprises amino acid residues Thr, Thr, His, Ala, His, Tyr, Ser, Asn, Asp, Ser, Leu, Ala and Leu at positions corresponding to amino acid positions 28, 30, 32, 33, 35, 50, 52, 55, 57, 99, 101, 102 and 103 of SEQ ID NO. 28, respectively. The pairwise sequence alignment is performed using scoring matrix: blosum62, gap opening penalty: 10, and gap extension penalty 0.2.

Hence, in one embodiment, an antibody library is provided for selecting tumor-targeting antibodies, wherein each antibody of the antibody library comprises a VH and a VL domain; wherein the VH domain of each antibody is a STn-binding VH domain and the amino acid sequence of the VH-domain in pairwise alignment with SEQ ID NO. 28 comprises amino acid residues Thr, Thr, His, Ala, His, Tyr, Ser, Asn, Asp, Ser, Leu, Ala and Leu at positions corresponding to amino acid positions 28, 30, 32, 33, 35, 50, 52, 55, 57, 99, 101, 102 and 103 of SEQ ID NO. 28, respectively; and the amino acid sequence of the VH domain has at least 60, 65, 70, 75, 80, 85, 90, 92, 94, 96, or 98% sequence homology to SEQ ID NO. 28, preferably at least 80%, more preferably at least 90%, most preferably at least 95% sequence homology to SEQ ID NO. 28.

In one embodiment, the first antibody domain is a mono- or bis-STn-binding VH domain, and wherein said VH-domain comprises an amino acid sequence having at least 90% sequence homology to SEQ ID NO. 28 and comprising amino acid residues (i), T28, T30, H32, A33, H35, Y50, S99, L101, A102 and L103, (ii), T28, T30, S52, N55, D57, L101, A102 and L103, or (iii) T28, T30, H32, A33, H35, Y50, S52, N55, D57, S99, L101, A102 and L103, with respect to SEQ ID NO. 28, preferably comprising amino acid residues T28, T30, H32, A33, H35, Y50, S52, N55, D57, S99, L101, A102 and L103, with respect to SEQ ID NO. 28.

The STn-template library is useful for screening for antibodies which bind combotopes comprising a STn epitope. The library may further be useful for screening for antibodies which bind combotopes comprising a Tn epitope, or a combination of Tn and STn epitope.

### III. Nucleic Acid Library Encoding Antibodies

In one aspect, the present invention provides a nucleic acid library encoding the antibody library disclosed herein.

All features and embodiments of the antibody library disclosed in section II equally applies to the nucleic acid library encoding antibodies disclosed in this section.

In one embodiment, the present invention provides a nucleic acid library encoding antibodies, wherein each of the nucleic acids in the library comprises
(i) a first nucleic acid sequence encoding an antibody domain which binds a carbohydrate epitope of a glycoprotein of the tumor cell, and
(ii) a second nucleic acid sequence selected from a repertoire of nucleic acids sequences comprising one or more nucleic acid sequences encoding an antibody domain that binds a peptide epitope of said glycoprotein of said tumor cell,

for in-vitro identification of a specific antibody from said library which specifically binds a tumor cell, wherein the peptide epitope of the glycoprotein of the tumor cell is associated with the carbohydrate epitope of said glycoprotein of said tumor cell, and wherein the specific antibody is specific for the combination of the carbohydrate epitope and the peptide epitope of said glycoproteine.

In one embodiment, the present invention provides a nucleic acid library encoding antibodies, wherein each of the nucleic acids in the library comprises
(i) a first nucleic acid sequence encoding a VH-domain which binds a carbohydrate epitope of a glycoprotein of the tumor cell, and
(ii) a second nucleic acid sequence selected from a repertoire of nucleic acids sequences comprising one or more nucleic acid sequences encoding a VL-domain that binds a peptide epitope of said glycoprotein of said tumor cell,

for in-vitro identification of a specific antibody from said library which specifically binds a tumor cell, wherein the peptide epitope of the glycoprotein of the tumor cell is associated with the carbohydrate epitope of said glycoprotein of said tumor cell, and wherein the specific antibody is specific for the combination of the carbohydrate epitope and the peptide epitope of said glycoproteine.

Provided herein are nucleic acid libraries comprising a plurality of nucleic acid sequences, wherein each nucleic acid sequence of the plurality of nucleic acid sequences encodes an amino acid sequence forming at least a part of an antibody as described herein.

Specifically, the present invention provides a nucleic acid library encoding a plurality of antibodies, wherein each of the plurality of nucleic acid sequences encoding antibodies comprises
(i) a first nucleic acid sequence encoding a VH-domain which binds a carbohydrate epitope on a glycoprotein of the one or more tumor cells, and
(ii) a second nucleic acid sequence selected from a repertoire of nucleic acid sequences comprising one or more nucleic acid sequences encoding a VL-domain, that binds a peptide epitope of said glycoproteins of said tumor cells,

for in-vitro identification of a specific antibody from said library which binds a tumor cell, wherein the peptide epitope of the glycoprotein of the tumor cells is associated with the carbohydrate epitope of said glycoprotein of said tumor cells, and wherein the specific antibody is specific for the combination of the carbohydrate epitope and the peptide epitope of the glycoprotein.

In one embodiment, the nucleic acid library comprises in the range of 10⁸-10⁹ non-identical clones, such as at least 10⁴, 10⁵, 10⁶, 10⁷, 10⁸, 10⁹, or more non-identical nucleic acids.

In a further embodiment, the first and the second nucleic acid sequences are linked by a nucleic sequence encoding a peptide linker connecting the encoded VH sequence with the encoded VL sequence. The peptide linker is discussed above. Such linkers are generally known to the skilled artisan.

Provided herein are further vector libraries comprising a nucleic acid library as described herein. Exemplary expression vectors for inserting nucleic acid libraries disclosed herein may comprise eukaryotic or prokaryotic expression vectors. Preferably, the nucleic acid library encoding antibodies are expressed using phage display technology.

Provided herein are further cell libraries comprising a nucleic acid library as described herein.

### IV. Method of Identifying Combotope Antibodies by Using Antibody Library of the Present Invention

In a further aspect, the present invention provides a method for identifying an antibody for targeting a tumor cell, comprising preparing an antibody library as disclosed herein, and screening said library to identify one or more tumor targeting antibodies.

In one embodiment, the antibody library is prepared as a phage displayed library, and the screening comprises biopanning of the antibody library using a specific tumor glycopeptides or the intact glycoprotein.

In a further embodiment, the method further comprises isolating the tumor targeting specific antibody, and optionally purifying the antibody. Antibody isolation and purification may be done by any common method recognized by a person skilled in the art.

In one embodiment, the process for identifying and isolating a tumor-specific antibody comprises the steps
(a) preparing an antibody library as disclosed herein, and
(b) identifying antibody candidates specific for target glycoprotein antigens from the library by binding assay(s).

In one embodiment, the identification of tumor cell specific antibody candidates comprises biopanning of the antibody library using a (specific) tumor glycopeptide or glycoprotein of said tumor cell, preferably O-glycosylated peptides or proteins, such as a Tn-mucin or other O-glycosylated proteins having mucin-like motifs, as a purified peptide/protein or expressed on cell surfaces/tissues.

The antibody library may be a phage display library, yeast display library, ribosomal display library, or similar, as recognized by a person skilled in the art.

In one preferred embodiment, the antibody library is a phage display library.

In one embodiment, the antibody library is a phage display library prepared by a method comprising the steps 1) mRNA isolation from a spleen, 2) cDNA synthesis from said mRNA, 3a) amplification from said cDNA using a specific set of primers to obtain a first nucleic acid sequence encoding the VH domain, 3b) application from said cDNA using a mix of primers to obtain multiple nucleic acid sequences encoding the repertoire of VL domains, 4) assembly of the first nucleic acid sequence encoding the VH-domain and a second nucleic acid sequence from the multiple nucleic acid sequences encoding the VL-domain repertoire, to form a joint construct, 5) insertion of the construct into a phagemid vector, 6) insertion of the phagemid vector comprising the construct into E. coli to produce a bacterial library, 7) using the bacterial library for infection of phages to produce the phage display library.

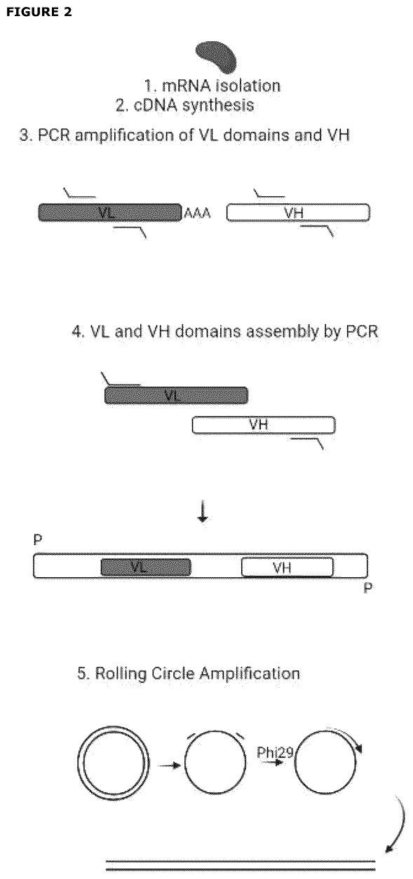

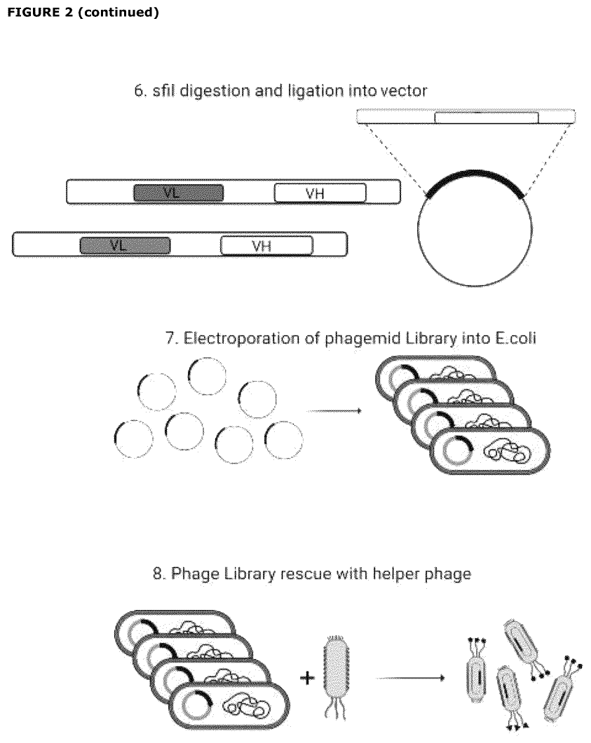

As a non-limiting example, such phage display library may be prepared as illustrated in FIG. 2, comprising 1) mRNA isolation from mouse spleen. 2) cDNA synthesis with reverse transcriptase using random hexamers. 3) PCR amplification from cDNA template to obtain the VH domain using a specific set primers, and the repertoire of VL domains using a mix of VL primers. 4) PCR assembly of VL-domain repertoire and specific VH-domain using 5′ phosphorylated outer primers. 5) Rolling circle amplification where phosphorylated scFv genes are ligated into circular DNA, dsDNA is denatured, random hexamers are annealed and Phi29 polymerase amplifies the circular fragments into long linear concatemers. 6) Amplified extended scFv genes are digested by sfiI restriction enzyme and ligated to Sfil and rSAP treated phagemid vector pAK100. 7) Pool of phagemids containing scFv genes are electroporated to TG1 E. coli cells. 8) Growth of Bacterial Library containing the different phagemids and infection with helper phage VCSM13 to produce complete phages displaying scFvs on their pIII coat protein.

Selections from the antibody library, such as from the phage display library, may be performed by several rounds of interrogation (panning) with immobilized biotinylated target antigen (eg. bisTn-MUC1 glycoprotein) on streptavidin-coated magnetic beads.

After each round of selection, phages are eluted, amplified and precipitated. Removing extraneous phage antibodies by absorption against non-targets (negative binders), naked beads, plastics, proteins, peptides or normal human cells may also be performed as needed. Sequencing (NGS) of enriched phages after each round of panning provides a fingerprint of VL-domain antibody sequences corresponding to target antigen structure and peptide sequence. Polyclonal phage ELISA may be used to confirm enrichments for target binder (e.g bis Tn-MUC1 target protein/peptide). Phage pools may then be converted to soluble scFvs and expressed as individual scFvs. Expression of the scFvs in the supernatant may be assessed with dot blot analysis.

Screening for tumor-specific scFv clones in said phage library may be done using a ELISA binding assay, glycoprotein/peptide microarray and biolayer interferometry (OCTET) against target protein/peptide and control proteins/peptides (non targets), provided scFv antibodies targeting the selected glycoprotein antigen with high specificity and affinity.

Binding (FACS) of selected scFv to tumor cells expressing the target glycoprotein antigen, such as breast adenocarcinoma cell lines MCF7, MDA-MD-231 COSMC KO may further be used to confirm tumor specificity.

In one embodiment, the present invention provides a method as disclosed herein, wherein the antibody library is a phage displayed library, and the screening comprises biopanning of the antibody library using a specific tumor glycopeptide, such as Tn-MUC1, Tn-CD43, Tn-MUC4, Tn-MUC16, etc. Several different Tn-tumor target are—as a non-limiting example—disclosed in the review by Kudelka et al 2015.

### V. Epitope Targets

The present invention provides an antibody library and method for identifying specific antibodies against combined glycoside-peptide epitopes (combotobes) on specific glycoproteins, such as Tn, bis-Tn, STn or bis-STn epitopes on specific cancer cells.

Provided herein are glycoside-peptide epitope binding antibodies which may have therapeutic effects due to their ability to specifically bind to specific glycoproteins on for example cancer cells. Preferably, the antibody library provided herein facilitates identification of an antibody that may be used to identify (diagnose) or treat a disease or disorder, such as cancer.

Provided herein are methods for treatment of proliferative disorders. Further provided herein are methods for treatment of a proliferative disorder, wherein the proliferative disorder is cancer, comprising identifying and isolating anti-cancer cell antibodies by screening an antibody library of the present invention for antibodies having high specificity for said cancer cells, and administering to a subject diagnosed with said cancer disease the antibody identified as described herein. A particular method of treatment involves the use of the combotrope antibodies of the present invention in loading natural killer (NK) cells with the specific antibodies for targeting the NK-cells to the target cells, e.g. cancer cells. The NK-cells may be harvested from the patient to be treated prior to the loading with the antibodies or provided as donor NK-cells. The loading may be in the form of the antibody or antibodies per se or as (a) nucleotide sequence(s) encoding the specific antibody/antibodies. In another method, the specific antibodies are linked to a cell toxin or a non-toxic precursor thereof or a similar cytotoxic effector molecule of cell death. The skilled artisar would readily know which effector molecules could be useful. Further provided herein are methods for treatment of a proliferative disorder wherein the cancer is selected from lung, head and neck squamous cell, colorectal, melanoma, liver, classical Hodgkin lymphoma, kidney, gastric, cervical, merkel cell, B-cell lymphoma, and bladder cancer. In a preferred embodiment, the cancer is a solid tumor.

Aspects of the invention include administering any one of the specific antibodies identified as described herein to a subject identified as having aberrant/truncated O-glycosylation (e.g. trucated O-glycosylation of MUC1 protein) as compared to a reference level, (e.g. level in a non-cancerous cell). In one embodiment, the truncated O-glysylation is selected from Tn and STn antigens, such as Tn-MUC1.

In one embodiment, the present invention provides combotope antibodies as disclosed herein for use in treatment of a disease associated with aberrant/truncated O-glycosylation. In one embodiment, the present invention provides combotope antibodies as disclosed herein for use in treatment of a disease associated with Tn and/or STn antigens. In one preferred embodiment, the present invention provide combotope antibodies as disclosed herein for use in treatment of cancer.

In another aspect, the disclosure features methods that include administering any one of the specific antibodies identified as described herein, or a composition comprising such antibody, e.g. a cell composition, antibody-drug conjugate, or antibody-radioisotope conjugate) to a subject in need thereof, said subject having, or identified or diagnosed as having a cancer characterized by hypoglycosylation of peptide epitopes in the cancer cells (e.g., pancreatic cancer, epithelial cancer, breast cancer, colon cancer, lung cancer, ovarian cancer, or epithelial adenocarcinoma).

Other embodiments include using a specific antibody identified as described herein in testing for the presence of cancer in a subject, for example as a or part of a test kit

In some embodiments, the libraries of the present invention comprise antibodies that are adapted to the species of an intended therapeutic target. Generally, these methods include “mammalization”. In some instances, the mammal is mouse, rat, equine, sheep, cow, primate (e.g., chimpanzee, baboon, gorilla, orangutan, monkey), dog, cat, pig, donkey, rabbit, and human. Preferably, the antibodies are intended for human therapeutic targets, and therefore humanized.

### VI. Use of the Identified Antibodies

Tumor-specific antibodies are used in immuno-oncology to target cancer cells and activate the immune system to attack these cells. They can work by directly binding to cancer cells and triggering an immune response, or by targeting molecules on cancer cells that suppress the immune response. This can lead to increased tumor cell death and/or slower tumor growth. Tumor-specific mAbs are often used in combination with other immune-based therapies, such as immune checkpoint inhibitors or CAR-T cell therapy, to enhance the anti-tumor immune response. Tumor-specific monoclonal antibodies are also used in antibody-drug conjugates (ADCs) to deliver a cytotoxic drug directly to cancer cells. The mAb in the ADC is designed to recognize and bind to a specific protein on the surface of cancer cells, and once bound, the cytotoxic drug is released to kill the cancer cell. The advantage of using an ADC is that it can selectively deliver the drug to cancer cells, minimizing the damage to healthy cells. Some examples of ADCs that use tumor-specific mAbs include trastuzumab emtansine (T-DM1) for HER2-positive breast cancer and inotuzumab ozogamicin for acute lymphoblastic leukemia.

In one embodiment, the antibodies of the present invention—i.e. antibodies identified using the antibody library of the present invention—are used to target cancer cells, such as to activate the immune system to attack the cancer cells.

Hence, in one aspect, the present invention provides an antibody as disclosed herein for use in treatment and/or prevention of cancer. In some instances, the cancer is lung, head and neck squamous cell, colorectal, melanoma, liver, classical Hodgkin lymphoma, kidney, gastric, cervical, merkel cell, B-cell lymphoma, or bladder cancer. In a preferred embodiment, the cancer is a solid tumor.

In one embodiment, the antibodies of the present invention are used in combination with other immune-based therapies, such as immune checkpoint inhibitors and/or CAR-T cell therapy, to enhance the anti-tumor immune response.

In one embodiment, the antibodies of the present invention is used in antibody-drug conjugates (ADCs), such as to deliver a cytotoxic drug directly to cancer cells.

In another aspect, the present invention provides a method of treating a cancer comprising administering a formulation comprising at least one specific antibody as disclosed herein to a patient in need thereof.

In one embodiment, the the antibody administered is conjugated to a cytotoxic moiety or loaded into a NK-cell, such as the patients own NK-cells, for being presented on the surface thereof.

Specific antibodies for use in treatment of cancer may be selected from a list of antibodies, wherein all antibodies comprise
(i) a VH-domain which binds a Tn- and/or STn-carbohydrate epitope of a glycoprotein of the tumor cell, and
(ii) a VL-domain which binds a peptide epitope of said glycoprotein of said tumor cell,

and wherein the antibody is specific for a combination of said carbohydrate epitope and said peptide epitope of said glycoprotein,

but wherein the list of antibodies does not 5E5, 5F7, 2D9.

In one embodiment, the antibody administered to the patient in treatment of cancer comprises
(i) a VH domain comprising an amino acid sequence having at least 90% sequence homology to SEQ ID NO. 1, and amino acid residues H32, A33, H35, Y50, and S99 and/or S52, N55, and D57, with respect to SEQ ID NO. 1, and (ii) a VL domain comprising an amino acid sequence selected from SEQ ID NO. 9-21.

In one embodiment, the antibody administered to the patient in treatment of cancer comprises
(I) a VH domain comprising an amino acid sequence having at least 90% sequence homology to SEQ ID NO. 28, and comprising amino acid residues (i), T28, T30, H32, A33, H35, Y50, S99, L101, A102 and L103, (ii), T28, T30, S52, N55, D57, L101, A102 and L103, or (iii) T28, T30, H32, A33, H35, Y50, S52, N55, D57, S99, L101, A102 and L103, with respect to SEQ ID NO. 28, and (II) a VL domain comprising an amino acid sequence selected from SEQ ID No. 22-23.

In one embodiment, the antibody administered to the patient comprises
(i) a first VH domain comprising an amino acid sequence having at least 90% sequence homology to SEQ ID NO. 1, and amino acid residues H32, A33, H35, Y50, S52, N55, D57, and S99, with respect to SEQ ID NO. 1, and (ii) a first VL domain comprising an amino acid sequence selected from SEQ ID NO. 9-21; or
(I) a second VH domain comprising an amino acid sequence having at least 90% sequence homology to SEQ ID NO. 28, and comprising amino acid residues T28, T30, H32, A33, H35, Y50, S52, N55, D57, S99, L101, A102 and L103, with respect to SEQ ID NO. 28, and (II) a second VL domain comprising an amino acid sequence selected from SEQ ID No. 22-23.

In another aspect, the present invention concerns diagnostics. Specifically, a Tn- or STn-binding monoclonal antibody as disclosed herein can be utilized as a diagnostic tool for cancer in a subject by targeting the Tn/STn antigen, a specific carbohydrate structure found on the surface of many cancer cells but rarely present in normal cells. The subject may be a human or an animal. The process begins with the administration of the Tn- or STn-binding mAb, which has been designed to specifically recognize and bind to the Tn- or STn-antigen. Once administered, the mAb circulates through the body and binds to the Tn- or STn-antigens expressed on the surface of cancer cells. This binding can be detected and visualized using various imaging techniques, such as PET, MRI, or fluorescence imaging, depending on the label attached to the mAb. The presence and distribution of the Tn/STn-antigen-mAb complexes in the body can then be analyzed to determine the presence, extent, and possibly the type of cancer. This method offers a targeted approach to cancer diagnosis, potentially allowing for earlier detection and a more precise understanding of the cancer's location and spread, which is crucial for effective treatment planning. Combotope antibodies of the present invention may be used in such diagnostics approach.

### VII. Humanization of Antibodies

As mentioned previously, the antibodies of the present invention are preferably humanized. This may be done is several different ways as acknowledged by a person skilled in the art.

A non-limiting example of such humanization of antibodies comprises the following steps:
1) Identification of the mouse monoclonal antibody: The first step in humanizing a mouse monoclonal antibody is to identify an antibody with the desired specificity and affinity. This is typically done by screening a large library of mouse monoclonal antibodies using techniques such as ELISA or flow cytometry.
2) Analysis of the antibody structure: Once a mouse monoclonal antibody with the desired specificity and affinity is identified, its structure is analyzed to identify the regions responsible for its antigen-binding properties. These regions are typically located in the variable regions of the antibody, which are highly diverse and are responsible for recognizing and binding to specific antigens.
3) Selection of a human antibody framework: A human antibody framework is selected based on its structural similarity to the mouse antibody framework. This is important because it ensures that the humanized antibody retains the overall structure and stability of the original antibody.
4) Replacement of the antigen-binding regions: The mouse-derived antigen-binding regions, also known as complementarity-determining regions (CDRs), are replaced with human-derived CDRs while retaining the overall structure of the antibody. This is done using genetic engineering techniques such as PCR, cloning, and site-directed mutagenesis.
5) Testing of the humanized antibody: Once the humanized antibody is produced, it is tested for its specificity, affinity, and functionality. This is typically done using techniques such as ELISA, flow cytometry, and Western blotting. The humanized antibody is also tested for its immunogenicity, which is its ability to trigger an immune response in humans. If the humanized antibody is found to be safe and effective, it can be further developed for use in human therapies.

### VIII. Method of Identifying Glycopeptide Targets

In a further aspect, the present invention provides a method for identifying a glycopeptide target, said target comprising a Tn and/or STn epitope, such as a glycopeptide target of a cancer cell. The library of the present invention is used for the identification of such glycopeptide targets by for example immunoprecipitation and mass spectrometry: This approach involves incubating the phage display antibody library with a cell lysate or tissue sample and allowing the antibody to bind to its target protein. The antibody-protein complex is then isolated by immunoprecipitation and subjected to mass spectrometry analysis to identify the protein. Another example is Protein microarray: Protein microarrays are arrays of immobilized antibodies that can be used to identify protein targets of antibodies. By incubating the phage display antibody library array with a cell lysate or tissue sample, and detecting binding, it is possible to identify the target protein.

Hence, in one embodiment, the present invention discloses a method for identifying a glycopeptide target, said target comprising a Tn and/or STn epitope and a peptide target, such as a glycopeptide target of a cancer cell, said method comprising the steps of
i) preparing an antibody library as disclosed herein, and
ii) incubating said antibody library with a sample comprising the glycopeptide target,
iii) analyzing one or more antibody-peptide complexes obtained from step ii) to identify the amino acid sequence of the peptide epitope of said glycopeptide target.

In a preferred embodiment, the sample is a cell or tissue sample, such as lysed cells or tissues. The antibody library is preferably prepared as a phage display library, and the glycopeptides targets may be identified by analysis of the antibody-glycopeptide complexes using mass spectrometry analysis, or other similar method as recognized by a person skilled in the art.

### IX. Determining VH- and VL-Domain Specificity

As disclosed herein, the VH-domain of the combotope antibody of the present invention binds a carbohydrate epitope of a glycoprotein of the cancer cell, while the VL-domain of the combotope antibody binds a peptide epitope of the same glycoprotein associated with the carbohydrate epitope.

By structural characterization, such as using X-ray crystallography (as described herein in example 1), a person skilled in the art is able to identify whether a VH-domain may be characterized as a carbohydrate epitope binding VH domain, and further whether a VL-domain may be characterized as a peptide epitope binding VL domain.

Another way of identifying a VH domain is by sequence analysis (as described herein in example 3.3). Identification of whether a sequence is a VH domain may be done by alignment of the sequence in question with (a) G2D11 VH domain SEQ ID NO.: 1, to check whether the amino acid sequence in question comprises amino acid residues positions corresponding to H32, A33, H35, Y50, S52, N55, D57, and S99, with respect to SEQ ID NO.:1—i.e. whether the sequence in question have the key residues needed for the VH domain functionality of Tn binding; or with (b) SEQ ID NO. 28 to check whether the amino acid sequence in question comprises amino acid residues position corresponding to T28, T30, H32, A33, H35, Y50, S52, N55, D57, S99, L101, A102 and L103, with respect to SEQ ID NO. 28—i.e. whether the sequence in question have the key residues needed for the VH domain functionality of STn binding. At least three amino acids corresponding to the above mentioned amino acid residues need to be present in the VH domain for it to function as a VH domain binding Tn and/or STn.

For the VH-domain to be versatile, it is important that the VH-domain does not contribute to or interfere with binding any peptide epitopes on the glycoprotein, which means that binding of the VH-domain to the carbohydrate epitope is not influenced, interfered or affected by any peptide epitopes on the glycoprotein. Only in this way, the VH-domain can be freely be combined with any VL-domain of choice for a “clean” binding without any disturbing binding between the VH-domain of the antibody and a peptide epitope, which unwanted binding may distort the result—in e.g. diagnosis or specific drug delivery, targeting a specific tumor glycoform (Tn or STn) on a given protein.

For the VL-domain, this is discovered as disclosed herein, and will be unique to each target. The identified VL domains have been (1) evaluated based on specificity and (2) correlated with other VL-domain sequences to identify common traits in the CDRs. X-ray may then confirm these traits.

### X. Preferred Numbered Embodiments of the Invention

Preferred embodiment 1. An antibody library for in-vitro identification of a specific antibody which binds a tumor cell, wherein each antibody in said library comprises
(i) a first antibody domain which binds a Tn- and/or STn-carbohydrate epitope of a glycoprotein of said tumor cell, and
(ii) a second antibody domain selected from a repertoire of second antibody domains, wherein the repertoire of second antibody domains comprises one or more second antibody domains which binds a peptide epitope of said glycoprotein of said tumor cell,

wherein said specific antibody is specific for a combination of said carbohydrate epitope and said peptide epitope of said glycoprotein.

Preferred embodiment 2. The antibody library according to preferred embodiment 1, wherein the carbohydrate epitope is covalently linked to the peptide epitope.

Preferred embodiment 3. The antibody library according to preferred embodiment 1 or 2, wherein the first antibody domain is a VH-domain, and wherein the second antibody domain is a VL-domain.

Preferred embodiment 4. The antibody library according to any one of preferred embodiments 1-3, wherein the first antibody domain is a VH-domain, wherein the second antibody domain is a VL-domain, and wherein the antibodies in the library are scFv wherein the VH-domain is linked to the VL-domain via a peptide linker.

Preferred embodiment 5. The antibody library according to any one of preferred embodiments 1-4, wherein the carbohydrate epitope is selected from mono-Tn, bis-Tn, mono-STn, bis-STn, and a combination of mono-Tn and mono-STn.

Preferred embodiment 6. The antibody library according to any one of preferred embodiments 1-5, wherein the first antibody domain is a mono- or bis-Tn-binding VH domain, and wherein said VH-domain comprises an amino acid sequence having at least 90% sequence homology to SEQ ID NO. 1 and comprising amino acid residues H32, A33, H35, Y50, and S99 and/or amino acid residues S52, N55, and D57, with respect to SEQ ID NO. 1.

Preferred embodiment 7. The antibody library according to any one of preferred embodiments 1-5, wherein the first antibody domain is a mono- or bis-STn-binding VH domain, and wherein said VH-domain comprises an amino acid sequence having at least 90% sequence homology to SEQ ID NO. 28 and comprising amino acid residues (i), T28, T30, H32, A33, H35, Y50, S99, L101, A102 and L103, (ii), T28, T30, S52, N55, D57, L101, A102 and L103, or (iii) T28, T30, H32, A33, H35, Y50, S52, N55, D57, S99, L101, A102 and L103, with respect to SEQ ID NO. 28.

Preferred embodiment 8. The antibody library according to any one of preferred embodiments 1-7, wherein the first antibody domain does not contribute to or interfere with binding any peptide epitope.

Preferred embodiment 9. The antibody library according to any one of preferred embodiments 1-8, wherein the repertoire of second antibody domains is generated from a naïve immune repertoire of VL-domains, an immunized immune repertoire of VL-domains, or a synthetically produced repertoire of VL-domains; preferably a naïve immune repertoire of VL-domains from an animal, such as a mouse or human.

Preferred embodiment 10. The antibody library according to any one of preferred embodiments 1-9, wherein the antibody library is a phage display library.

Preferred embodiment 11. A method for identifying an antibody for targeting a glycoprotein, comprising the steps of (i) preparing an antibody library according to any one of preferred embodiments 1-10, and (ii) screening said library to identify one or more tumor targeting antibodies.

Preferred embodiment 12. The method according to preferred embodiment 11, comprising biopanning of the antibody library using a glycopeptide or glycoprotein of said tumor cell, preferably an O-glycosylated peptide or protein, such as Tn-mucin or other O-glycosylated protein having mucin-like motif, wherein said glycopeptide or glycoprotein is used in purified form or expressed on a cell surface or tissue.

Preferred embodiment 13. The method according to preferred embodiment 11 or 12, wherein the antibody library is a phage display library prepared by a method comprising the steps 1) mRNA isolation from a spleen, 2) cDNA synthesis from said mRNA, 3a) amplification from said cDNA using a specific set of primers to obtain a first nucleic acid sequence encoding the VH domain, 3b) application from said cDNA using a mix of primers to obtain multiple nucleic acid sequences encoding the repertoire of VL domains, 4) assembly of the first nucleic acid sequence encoding the VH-domain and a second nucleic acid sequence from the multiple nucleic acid sequences encoding the VL-domain repertoire, to form a joint construct, 5) insertion of the construct into a phagemid vector, 6) insertion of the phagemid vector comprising the construct into E. coli to produce a bacterial library, 7) using the bacterial library for infection of phages to produce the phage display library.

Preferred embodiment 14. A method for identifying a glycopeptide target, said target comprising a Tn and/or STn epitope and a peptide epitope, such as a glycopeptide target of a cancer cell, said method comprising the steps of (i) preparing an antibody library according to any one of preferred embodiments 1-10, (ii) incubating the antibody library with a sample comprising the glycopeptide target, and (iii) analyzing one or more antibody-peptide complexes obtained from step (ii) to identify the amino acid sequence of the peptide epitope of said glycopeptide target.

Preferred embodiment 15. A specific tumor cell binding antibody, comprising
(i) a VH-domain which binds a Tn- and/or STn-carbohydrate epitope of a glycoprotein of the tumor cell, and
(ii) a VL-domain which binds a peptide epitope of said glycoprotein of said tumor cell,

wherein the antibody is specific for a combination of said carbohydrate epitope and said peptide epitope of said glycoprotein, with the provision that the antibody is not 5E5, 5F7 or 2D9.

Preferred embodiment 16. The antibody according to preferred embodiment 15, wherein the VH domain comprises
(i) a first amino acid sequence having at least 90% sequence homology to SEQ ID NO. 1, wherein the first amino acid sequence comprises amino acid residues H32, A33, H35, Y50, and S99 and/or amino acid residues S52, N55, and D57, with respect to SEQ ID NO. 1; or
(ii) a second amino acid sequence having at least 90% sequence homology to SEQ ID NO. 28, and wherein the second amino acid sequence comprises amino acid residues (i), T28, T30, H32, A33, H35, Y50, S99, L101, A102 and L103, (ii), T28, T30, S52, N55, D57, L101, A102 and L103, or (iii) T28, T30, H32, A33, H35, Y50, S52, N55, D57, S99, L101, A102 and L103, with respect to SEQ ID NO. 28.

Preferred embodiment 17. The antibody according to preferred embodiment 15 or 16, wherein the VL domain comprises an amino acid sequence selected from SEQ ID NO. 9-24.

Preferred embodiment 18. A method of treating a cancer in a subject comprising administering a formulation comprising at least one antibody according to any one of preferred embodiments 15-17 to a patient in need thereof.

Preferred embodiment 19. A method of diagnosing a cancerous condition in a subject comprising administering a formulation comprising at least one antibody according to any one of preferred embodiments 15-17 to the subject, and detecting the presence of an antigen-antibody complex comprising said at least one antibody according to any one of preferred embodiments 15-17 and a Tn- and/or STn-antigen.

### EXAMPLES

The following examples are set forth to illustrate more clearly the principle and practice of embodiments disclosed herein to those skilled in the art and are not to be construed as limiting the scope of any claimed embodiments.

All chemicals were supplied by Merck, Germany unless otherwise stated. All buffers, media, were dissolved in milli Q water (MQ) and autoclaved unless otherwise stated.

### Peptides

The peptides that were used in phage display selection, ELISA and Bio-layer interferometry (BLI) are shown in table 1. CD43 and IgA have been previously synthetized in house by solid phase peptide synthesis (SPPS) as described in Persson et al 2016. MUC1 peptides were either synthesized or purchased by Biosyntan, Germany.

TABLE 1
Peptide amino acid sequence used in phage display, ELISA and BLI
experiments. Amino acids in bold* is a GalNac residue. Amino acids in bold and
underlined is a sialylated GalNac residue.

| Peptide | | | SEQ |
|---|---|---|---|
| no | Protein | Sequence | ID NO. |
| 1 | MUC1 | Btn-Ahx-SAPDTRPAPGS*T*APPAHGVTSAPD-OH | 57 |
| 2 | MUC1 | Btn-Ahx-SAPDTRPAPGS*TAPPAHGVTSAPD-OH | 58 |
| 3 | MUC1 | Btn-Ahx-SAPDTRPAPGST*APPAHGVTSAPD-OH | 59 |
| 4 | MUC1 | Btn-Ahx-SAPDTRPAPGSTAPPAHGVTSAPD-OH | 60 |
| 5 | MUC1 | Biotin-Ahx-APPAHGVTSAPDT*RPAPGSTAPPAHGVTSA-OH | 61 |
| 6 | MUC1 | GS*T*AP | 62 |
| 7 | MUC1 | GST*AP | 63 |
| 8 | IgA1 | Btn-OEG-OEG-OEG- VPSTPPTPSPS*T*PPTPSPSA | 64 |
| | hinge | | |
| | region | | |
| 9 | CD43 | Btn-OEG-OEG-OEG- PLWTSIGAS*T*GSPLPPEPTTY | 65 |
| 10 | CD43 | Btn-OEG-OEG-OEG- PLWTSIGASTGSPLPPEPTTY | 66 |
| 11 | MUC1 | GSTAP | 67 |
| 12 | MUC1 | GSTAP | 68 |

Btn = biotin; Ahx = aminohexyl; OEG = Oligo-ethylenglycol

### Cell Lines

All cell lines were maintained at 37° C. in a 5% CO2 humidified incubator. MCF7, MDA-MD-231 WT and COMSC KO were maintained in DMEM+GlutaMax (Gibco, 32430-027) supplemented with 10% FBS (FisherScientific, 11550356), 1% penicillin-streptomycin (FischerScientific, 15140122) and 1 mM sodium pyruvate (Gibco, 11360). MDA-MB-231 WT and COSMC KO cells were kindly provided by Ulrich auf dem Keller. Jurkat cells were maintained in RPMI (Life Technologies, 32404014) supplemented with 10% FBS, 1% penicillin-streptomycin and 2 mM L-glutamine (Sigma, G7513). HEK293 cells were maintained in Freestyle media (Thermo Scientific, 15285885).

Broth Media, Plasmids and E. coli Strains

XL1-Blue electrocompetent cells were supplied by Agilent (Agilent, 200228). TG1 for phage display were kindly provided by Peter Kristensen from Aalborg University. Vectors pAK100 phagemid and pJB33 expression vector were kindly provided by Plunthum from University of Zurich, both with chloramphenicol antibiotic resistance. E. coli TG1 and XL1—blue electrocompetent cells were cultures in 2×YT broth media. Liquid media was supplemented with 25 μg/mL chloramphenicol and 2% glucose unless otherwise stated.

G2D11 VH chain and mutant in pTwist vector by Twist Biosciences.

### Softwares

GraphPad prism 9 was used for graph design and Biorender for image design. CLC Main workbench 8.0 software was used for sequence alignment.

### Example 1: Characterization of G2D11

G2D11 is a mouse derived anti-Tn-scFv mAb. ScFv consists of VH domain SEQ ID NO 1 and LV domain SEQ ID NO. 2 joined by pepide linker (GGGGS)₄.

### 1.1 Structural Characterization

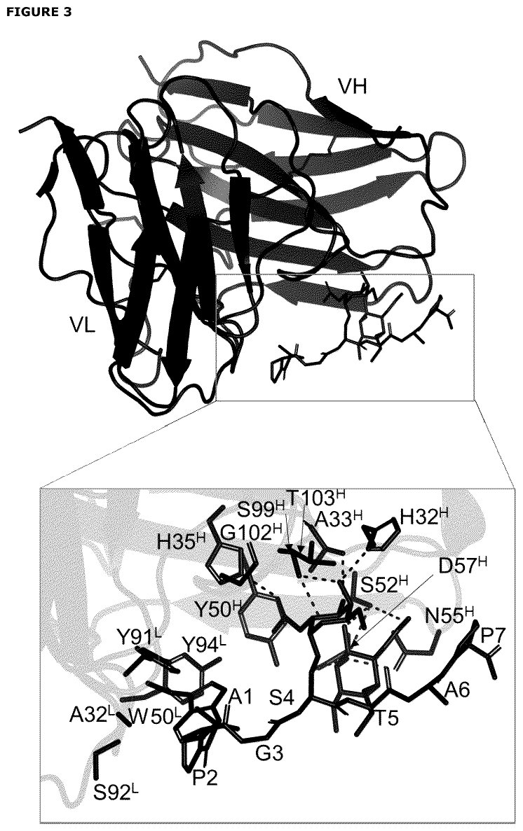

ScFv G2D11 crystals were prepared by the sitting drop technique and by using appropriate precipitant solutions. The resulting crystals were used to solve the structure at a resolution of 1.9 Å and interpret the density map (FIG. 3). Despite two molecules were present in the asymmetric unit and that contacted weakly between each other, analytical ultracentrifugation showed that this monomeric form behaved as a monomer either in the absence or presence of the bis-Tn-MUC1 peptide APGS*T*AP where * denotes a GalNAc moiety (SEQ ID NO.: 55). The glycopeptide laid within a surface groove formed by the light (L) and heavy (H) chains (hereafter VL and VH, respectively), and in particular the two GalNAc moieties were recognized by residues from the three hypervariable regions of the VH (FIG. 3).

With the exception of the OH6, all Ser-bound GalNAc hydroxyl groups were engaged in hydrogen bonds. In detail, hydroxyl group OH3 interacted with the NH group of Ala33H and OH4 with the side chains of His32H and Ser99H. The endocyclic oxygen of the sugar was engaged in hydrogen bonding with Ser99H. The carbonyl group of GalNAc was involved in a hydrogen bond with the side chain of His35H and the methyl group was engaged in a CH-n stacking interaction with His50H.

These interactions for G2D11 were found conserved when compared to a previous structure solved using the scFv-5E5 complexed to a mono-Tn-MUC1 peptide (APGST*AP) (Macias-León et al 2020). In addition for 5E5, a further CH-π interaction between Phe102^{H} and the Thr5 methyl group was visualized (Macías-León et al 2020). Phe102 helps direct VH into one of the two GalNAcs. The implications of this Phe102 is that 5E5 has a less flexible approach to Tn, it is restricted to Tn-Thr, and can only bind in a certain way. This further suggests that 5E5 prefers monoTn instead of bisTn.

Strikingly, the Thr-bound GalNAc was also intimately recognized by the scFv-G2D11. In this case, Ser52^{H} was engaged in hydrogen bond interactions with the carbonyl group, OH3 and OH4, while Asn55 and Asp57 side chains interacted with OH4. G2D11 is more open and can easier tolerate binding any combination TnThr/TnSer, TnSer/TnThr, TnSer/TnSer, TnThr/TnThr.

The recognition by G2D11 for GalNac binding sites was supported by the absence of binding of a triple mutant (H32A^{H}-H35A^{H}-S52A^{H}) or the double mutant (S99A^{H}-S52A^{H}) towards bis-Tn-peptides (data not shown).

Interestingly, scFv-G2D11 did not recognize the peptide sequence though the Ala1 and Pro2 were surrounded by aromatic residues of the VL (FIG. 3). On the contrary, the scFv-5E5 VL recognized the peptide by a hydrogen bond between Tyr98^{L} and Pro7 backbones and a CH-7t interaction between Tyr100^{L} and Pro7 (Macías-León et al 2020).

### 1.2 Sequence Alignment

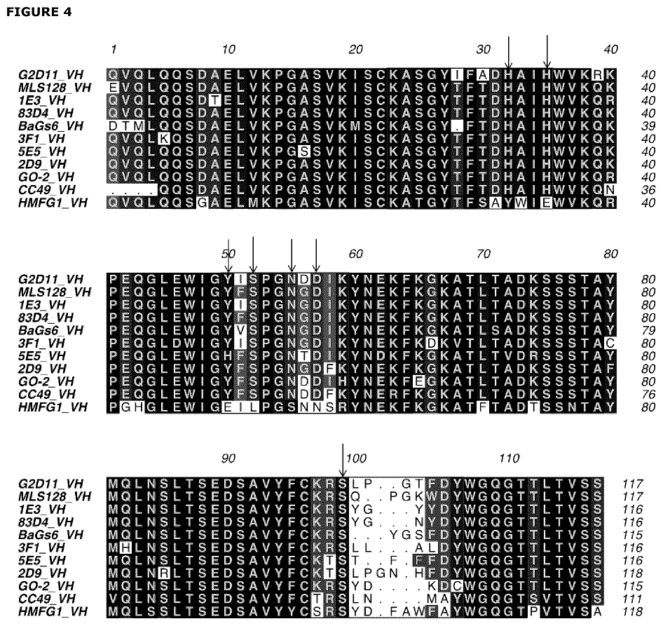

G2D11 VH-domain was aligned with other known anti Tn-antibody VH-domains using CLUSTALW (using standard settings for multiple alignment parameters—i.e. scoring matrix: blosum62, gap opening penalty: 10, and gap extension penalty 0.2). Based on this alignment, conserved amino acid residues in the CDR1, CDR2 and CDR3 regions relevant for the functionality of the VH-chain (i.e. binding the GalNac) were identified, as illustrated in FIG. 4 by the arrows.

Specifically, with reference to G2D11 (SEQ ID NO. 1), amino acid residues H32, A33, and H35 in CDR1, amino acid residues Y50, S52, N55, and D57 in the CDR2, and also amino acid residue S99 in the CDR3 should preferably be conserved for the VH domain.

### Example 2: Tn-Template Phage Display Libraries Conceptualization

Based on structural the VH-domain X-ray data and the observation that bisTn-binding was due to the VH-domain (as disclosed in Example 1), an antibody library was conceptualized, wherein each antibody comprised the VH chain of the previously identified scFv G2D11, providing recognition support for the glycoside part of the antigen, while the VL-domain was variable originating from naïve mice, creating a scFv phage display library, termed as Tn-template library, which can be screened for a specific scFv, of which the VL-domain would provide recognition support to the underlying peptide antigen within the combotope.

### 2.1 Phage Display Library Construction

Wild type BALB/c mice were euthanized, the spleens were removed and directly stored in −80° C. in RNAlater RNA stabilization reagent until use. The RNA was isolated from the spleen using a gentleMACS Dissociator and miRNeasy kit (Qiagen) according to the manufacture's instruction. 1 μg of RNA was used for cDNA synthesis with random hexamer primers (FisherScientific, 10609275) and SuperScript IV Reverse Transcriptase (Invitrogen, 18090010). The constant VH gene as well as the VL antibody specific genes were amplified by PCR using Q5 Hot Start High-Fidelity DNA Polymerase (NEB M0494S). The primers that were used are found in the sequence listing (SEQ ID NOs. 31-32). VL and VH genes were gel extracted and assembled with 5′ phosphorylated outer primers to allow the rolling circle amplification in the next step. RCA improves restriction enzyme (SfiI) cutting of the scFv genes. The assembled scFv fragments were sub-cloned in the Sfil-digested phagemid vector pAK100 using Electroligase (NEB M0369) for 16 h at 16° C./25° C. The phagemid pool with a variety of scFv fragments was electroporated in XL1-Blue electrocompetent cells (Agilent, 200228). Cells were recovered in SOC media, incubated for 1 h at 37° C. at 220 rpm, plated on selective media agar plates and incubated overnight at 30° C. Colonies were scraped off with cold 2×YT, supplemented with 25% v/v glycerol and stored at −80° C. For phage rescue, cells were infected with VCSM13 helper phage yielding 1013 phages/mL that were used in the biopanning. The primers that were used are found in the sequence listing (SEQ ID NOs. 33-49).

2.2 Solid Phase scFv Antibody Selection, Phage Rescue and Production

Three rounds of selection were performed using streptavidin M-280 Dynabeads (Invitrogen 11205D) and all incubations were performed at RT on rotation unless otherwise stated.

Beads were blocked with 5% BSA in PBST (PBS with 0.05% Tween 20) for 1 h. After 3 washes with PBST, 100 nM of biotinylated peptides (50 nM in the last round) diluted in 3% BSA in PBST were coupled with the beads for 2 h. Phage library (1012 phages/mL) were pre-selected against naked beads for 1 h and then transferred for positive selection against the target antigen for 2 h. Unbound phages were removed by washing 3 times with 3% BSA in PBST, 3 times with PBST and 3 times with PBS. Bound phages were eluted with 1 mg/mL of freshly prepared trypsin solution and were allowed to infect exponentially growing E. coli TG1 in for 30 min at 37° C. Infected bacteria were spread on agar plates and incubated overnight at 30° C. Colonies were scraped with medium, homogenized and 1:1000 of the homogenous mixture was inoculated in liquid media and were grown at 37° C. at 220 rpm until they reach OD600=0.4-0.5. Subsequently, they were infected with VCSM13 helper phage (109 phages/mL) for 30 min at 37° C. Bacteria were spun and pellet was resuspended in liquid media without glucose but supplemented with antibiotics and isopropyl β-D-1-thiogalactopyranoside (IPTG in 1:1000 dilution) to induce phage production. Overnight cultures were centrifuged to remove bacteria pellet and phages were precipitated form the supernatant by adding ice cold PEG/NaCl (20% w/v PEG6000, 2.5 M NaCl) in 1:4 ratio. After 1 h incubation on ice, precipitated phages were spun at 10,800×g for 30 min followed by a centrifugation at 5,000×g for 5 min. phage pellet was resuspended in 1 mL of cold PBS and was further centrifuged at 13,000×g for 10 min to remove any cell debris. Concentration was measured spectrophotometrically at 269/320 nm according to the following equation: Virions/ml=(A269−A320)×6×1016/number of bases per virion. The precipitated phages were used in the subsequent rounds of selections.

For the selections with the STn library, peptides were immobilized on NHS beads (Fisher Scientific, 88827). First beads were washed once with ice cold 1 M hydrochloric acid (HCl). 100 nM of peptides were diluted in print buffer and incubated with the beads for 2 h. two washed with 0.1 M glycine pH=2 followed and then blocking of 1 h with 3 M ethanolamine for 1 h. Phage library incubation, washes and trypsin elution followed as described previously.

2.3 Subcloning, Expression and scFv Screening

After three rounds of selection, polyclonal phagemids with the different scFv fragments were purified with the GeneJet Plamsid Miniprep Kit (Thermo Fischer, K0503) according to the protocol, digested with Sfil restriction enzyme for 20 min at 50° C. and ligated in the pJB33 expression vector using T4 electroligase for 1 h at 65° C. The pool of the different constructs was electroporated in XL1-Blue electrocompetent cells, cells were recovered in SOC media, incubated for 1 h at 220 rpm at 37° C. and then cells were spread on agar plates and incubated overnight at 37° C. 62 individual colonies were picked and incubated overnight at 37° C. in 96 U-bottom well plates. Overnight cultures were inoculated in fresh media without glucose and were incubated for 4 h at 37° C. at 220 rpm before IPTG induction (0.5 mM final concentration) and overnight cultivation at 30° C., at 800 rpm in a humidified incubator. Cells were pelleted by centrifugation at 3,000×g for 10 min and the supernatant was used for positive hint binding with ELISA. For clone sequencing analysis, plasmid DNA was purified from each individual clone and sent for Sanger sequencing in Macrogen using M13R custom designed primer. CLC Main workbench 8.0 software was used for sequence alignment.

2.4 Soluble scFv Production and Purification

All periplasmic protein extraction steps were performed on ice for 1 h and all the centrifugations at 4° C. Selected clones were grown from the glycerol stocks overnight at 37° C., at 220 rpm, in 2×YT supplemented with 2% glucose and 25 μg/ml chloramphenicol. Overnight cultures were diluted in fresh media and cultured until exponential phase before induction with 1 mM IPTG and overnight incubation at 20° C., at 220 rpm. Bacteria were harvested at 6,000×g for 10 min and pellet was resuspended in ice-cold 100 mM Tris, 20% w/v sucrose solution with EDTA-free protease inhibitor cocktail (ThemroFischer, A32965), pH 8. After centrifugation at 8,000×g for 10 min, pellet was resuspended in ice-cold 5 mM MgSO4 in MQ solution. Pellet was centrifuged at 8,000×g for 10 min and the 2 fractions were pooled together and centrifuged at 12,000×g for 60 min to remove any cell debris.

Pooled fractions with the soluble scFv were filtered with 0.45 μm filter membrane, the supernatant was mixed with 4× equilibration buffer (100 mM Tris, 1.2 M NaCl, pH 8) in 3:1 ratio (v/v) and were incubated overnight with nickel-nitrilotriacetic acid (Ni-NTA) agarose beads (Qiagen, 30210) at 4° C. on rotation. Beads with the captured scFv were pelleted by centrifugation at 1,000×g for 2 min and were loaded on a pre-equilibrated affinity resin column (Thermo Scientific, 29920) with 10 column volumes (CV) of 1× equilibration buffer. Unbound proteins were washed away with 10 CV wash buffer (1× equilibration buffer with 10 mM imidazole, pH 8) and bound scFv were eluted with 0.2 CV of elution buffer (1× equilibration buffer with 250 mM imidazole, pH 8). The elution step was repeated 2 more times. Eluted scFv antibodies were desalted followed by buffer exchange in PBS with Zeba spin desalting columns (Fisher Scientific, 89892) according to the manufacturer. Protein quantification was performed with BCA Protein assay kit (ThermoFischer, 23225) according to the protocol and purity was evaluated with SDS-PAGE.

### 2.5 Enzyme-Linked Immunosorbent Assay (ELISA)

All steps were performed at RT shaking unless otherwise stated. All washes between steps were done with PBST (PBS with 0.05% Tween20). 96-well Maxisorp plates (ThermoScientific, 10394751) were used for antigen immobilization in coating buffer (0.015M Na2CO3, 0.035 M NaHCO₃, pH 9.6). Streptavidin (NEB N7021S) was coated overnight at 4° C. at a concentration 4 times less of the antigen concentration. PLIP (0.5M NaCl, 0.003M KCl, 0.0015M KH2PO4, 0.0065 M Na2HPO4.2H2O, 1% w/v BSA, 1% Tween20) was used as blocking buffer for 1 h shaking. 3,3′, 5,5;-tetramethylbenzidine (TMB, Fisher Scientific 12617087) chromogen was used as a substrate for positive signal detection. The reaction stopped with 0.5 M H2SO4 and absorbance was measured at 450 nm on a VICTOR Nivo plate reader.

For the polyclonal phage ELISA, plates were incubated with polyclonal phages in serial dilutions in blocking buffer for 2 h followed by incubation with secondary antibody incubation for 1 h. Bound phages were detected with mouse monoclonal anti M13-HRP antibody (Nordic Biosite 58-11973-MM05T-H-100) at 1:10000 dilution.

For monoclonal scFv ELISA, antigen concentration was 50 nM. 50 uL of supernatant from the overnight culture of each clone was added per well. For antibody titration ELISA, antigen coating was at fixed concentration of 330 nM peptides and scFv were titrated 5-fold starting from 300 nM. Bound scFv were detected with a mouse monoclonal anti-His HRP (C-term) (Invitrogen 46-0707) at 1:2000 dilution.

### 2.6 Cell-Binding Assays

Cells were washed twice in FACS buffer (DPBS (Sigma-Aldrich, D8537) with 0.1% w/v BSA) before treatment with 100 mU/mL Clostridium perfringens neuraminidase (Sigma, N5631) for 30 min at 37° C. After 2 washes, cells were resuspended in 100 μL of FACS buffer and transferred in 96 U bottom well plate. Cells were stained for 2.5 μg/mL PNA (B-1075), 0.4 μg/mL VVA (B-1235), 1 μg/mL SNA (B-1305) and MAL I (B-1315) to evaluate neuraminidase efficiency. To detect positive binding against the target of interest, cells were stained with anti-MUC1 and anti-CD43 scFv antibodies at 5, 1.25, 0.3 and 0.08 μg/mL for 30 min on ice. Rabbit anti-MUC1 (HMFG2) (Abcam, ab245693) at 1 μg/mL and CD43-FITC (Miltenyi, 130-097-360) at 1:20 was used as a positive control. Cells were washed 2 times and stained with streptavidin Alexa Fluor 488 conjugated streptavidin (Invitrogen, S32354) at 1:1000 to detect lectin binding, anti His Alexa Fluor 647 conjugated (R&D IC050R) at 1:1000 to detect scFv binding, and goat ant-rabbit Alexa Fluor 647 (1:1000) for 20 min on ice in the dark. After 2 washes, cells were analysed on Miltenyi Biotech-MACS Quant 16. Data analysis was performed using FlowJo Version 10. All lectins were supplied by Vector Biolabs.

### 2.7 Biolayer Interferometry (BLI)

Steady state kinetics were determined using an Octet Red96 system. Samples and buffers were dispensed into polypropylene 96well black flat-bottom plates (Greiner Bio-One, 655209) at a final volume of 200 μL per well, and all measurements were performed at 30° C. with agitation at 1000 rpm. Prior to each assay, high streptavidin biosensor tips (SAX) (Sartorius, 18-5117) were prewetted in kinetics buffer (DPBS supplemented with 0.1% BSA and 0.02% Tween20) for at least 10 min followed by equilibration in kinetics buffer for 60 s. The streptavidin biosensor tips were loaded with the biotinylated target glycopeptide in kinetics buffer for 300 s, followed by an additional equilibration step of 100 s. Association of scFvs in a range of different concentrations was performed for 300 s. Finally, the dissociation was monitored with kinetics buffer for 300 s. The association and dissociation responses were processed with the Octet Software (Version 12). Interferometry data was globally fitted to a 2:1 model calculating the affinities and rate constants.

### 2.8 VL Diversity Sequencing

To follow phage enrichment during rounds of panning, the following LV diversity screening was performed. scFv VL-sequences that are related to the target peptide will appear with higher frequencies (enriched) confirming that phages with scFv are binding to the selected targets. These data is then be used for comparison with sequences from selected clones.

Sequencing was performed with Oxford Nanopore Technology (ONT). After each selection round, bacteria were scraped from the agar plate and an aliquot of the homogenous suspension was used for DNA purification using the GeneJet Miniprep Kit according to the manufacture's protocol. For the unselected libraries, homogenous suspension of scraped bacteria were used for DNA purification using Nucleobond Xtra EF Plasmid purification (MACHEREY-NAGEL GmbH & Co, 740422.50M) according to the manufacture's protocol. The set of primers that were used are found in the sequence listing (SEQ ID NOs. 50-53). Three pg of plasmid DNA were used as input material. Nanopore sequencing and data analysis was performed according to Karst et al 2021 with the following modifcations: a 0.8×volume of AMPure XP beads was used for DNA clean-up after early and late PCR, all the DNA washes for the purification were performed with 80% ethanol. DNA was quantified using the Qubit dsHS DNA assay (Thermo Fisher Scientific). After late PCR, a 1% agarose gel was performed to verify the correct product size. Samples prepared for the R9 flow cell the SQK-LSK110 ligation sequencing kit protocol was used while samples prepared for the R10 flow cell the SQK-LSK114 ligation sequencing kit protocol was used. Samples run on a flow cell and sequencing was performed on a MinION Mk1B device for 72 h.

### Example 3: MUC1 as a Proof of Concept

MUC1 was used as proof of concept for the constructed libraries to identify binders against MUC1. Comparing the newly identified scFvs and the known mAbs in terms of sequence and antibody activity, the effectiveness and the functionality of both libraries was determined. The identified scFv were sequenced followed by VL chain analysis and characterized for their specificity on ELISA, cell binding assays and kinetic studies with BLI.

### 3.1 Phage Selection

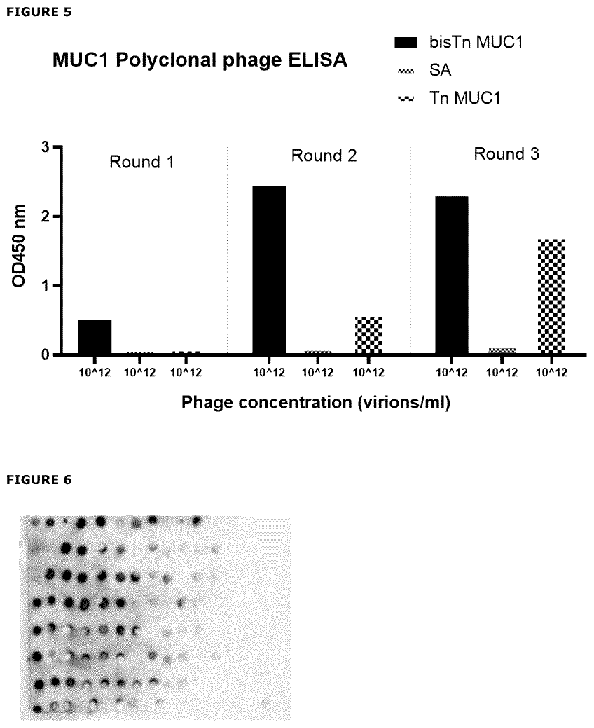

Tn template library, wherein each antibody in the library comprises G2D11 VH domain (SEQ ID NO. 1) was subjected to three rounds of selections by immobilizing target peptide 1 (see Table 1) on streptavidin-coated beads using target antigens. After each round of selection, phages were eluted, amplified and precipitated. Phage stocks after each round were titrated and analysed on polyclonal phage ELISA (FIG. 5). Polyclonal phage ELISA showed specific binder enrichment for MUC1 target peptide in every round with zero non-specific binders against streptavidin. Interestingly, in round two and three binders against peptide 3 (see Table 1) have been enriched and therefore the library can be potentially used to identify binders for peptides with one GalNac. In addition, nanopore sequencing after each biopanning round was performed to verify sequence enrichment specific for MUC1. From previous studies, based on the crystallography of 5E5 mAb (SEQ ID NO. 3+4) and the other known mAbs, CDR3 sequence and specifically the motif YXY in CDR3 is responsible for MUC1 peptide backbone binding. Based on that observation, the twenty most frequent CDR3 sequences were ranked and sequence enrichment was demonstrated (Table 2). In addition, the MUC1 specific motif YXY in CDR3 sequences was identified.

TABLE 2
Table lists the ten out of twenty CDR3 sequences in every round and in
bold are highlighted the MUC1 specific binding motif Tyr-X-Tyr. The values (%)
describe the number of reads of each CDR3 divided by the number of total reads
in each round.

| Rank | CDR3_VL* | Naive | 1^{st} round | 2^{nd} round | 3^{rd} round |
|---|---|---|---|---|---|
| 1 | QQYNSYPLT | 0.36 | 45.33 | 76.18 | 67.26 |
| 2 | QQYYSYPT | 0.01 | 1.89 | 1.61 | 10.67 |
| 3 | QQYSSYPLT | 0.16 | 10.83 | 9.01 | 5.86 |
| 4 | QQYYSYPLT | 0.00 | 0.44 | 3.93 | 5.76 |
| 5 | QQYNSYPLA | 0.06 | 1.72 | 3.75 | 3.10 |
| 6 | HQHYSTPPT | 0.00 | 0.91 | 1.52 | 3.07 |
| 7 | KQYNSYPLT | 0.00 | 1.22 | 2.14 | 2.44 |
| 8 | LQHWNYPLT | 0.00 | 1.22 | 0.27 | 0.51 |
| 9 | QQDYSSPLT | 0.02 | 0.10 | 0.18 | 0.51 |
| 10 | QQYNSFPLA | 0.00 | 0.24 | 0.27 | 0.19 |

*SEQ ID NOs 79-88 in sequence listing.

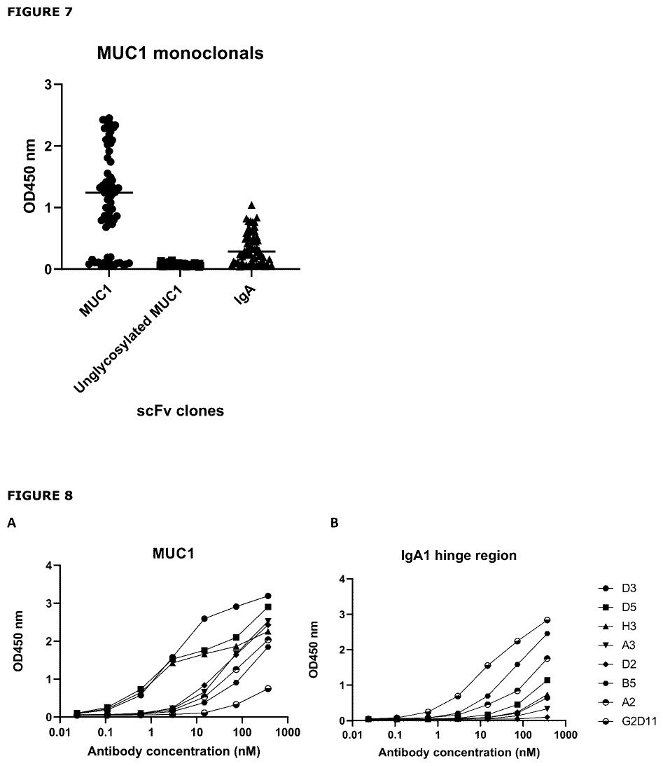

To specifically isolate binders for each target, DNA polyclonal phagemid pool after the third round was purified and the pool of different scFv genes were subcloned in the expression vector followed by individual scFv expression in a 96-well format. Expression of scFv in the supernatant was assessed with dot blot analysis (FIG. 6). Sixty-one clones were picked and screened with monoclonal ELISA against target peptide 1 (see Table 1), and control peptides 4 and 8 (FIG. 7). Peptide 4 was used to double confirm that with the Tn template library, mAbs against the backbone cannot be selected. The control peptide that was used in this study is IgA (see Table 1) that is produced in mucosal membranes and plays a significant role in their immunity. It has N- and O-linked glycosylation sites and is involved in a number of pathological conditions such as IgA deficiency and IgA nephropathy. Clones that showed no reactivity against the control peptides were selected for further characterization. In addition, sequences of the sixty one clones with sanger sequencing were obtained and alignment with VL sequences of 5E5 and 2D9 showed the differences in the CDR of the VL chains. Key binding features as presented in Table 2 were also present in VL-sequences from 5E5 and 2D9 further corborating their importance for interactions with the peptide backbone and determination of specificities.

3.2 Soluble scFv Expression and Evaluation

Based on monoclonal scFv ELISA specificity and VL sequence comparison, 10 clones were selected for purification. Of these ten clones, three clones (D3, A3 and D2) were fully evaluated and are represented by SEQ ID NOs. 9-11 in the sequences listing. Only their VL-domain is listed for the scFv. VH domain is same for the scFV of all clones—i.e. the VH of G2D11 (SEQ ID NO 1). The VL and VH are joined by the peptide linker (GGGS)₅.

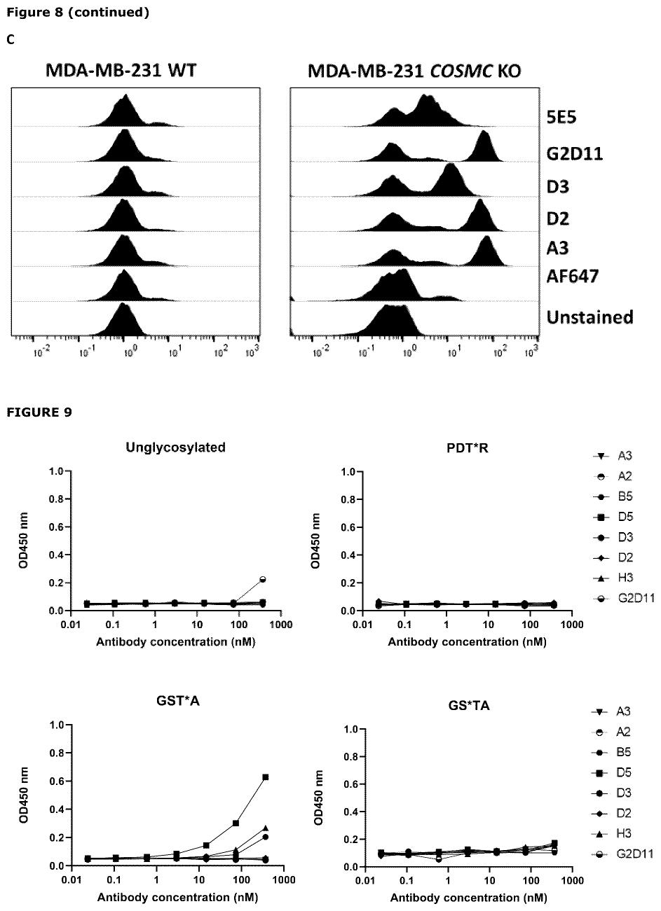

The selected clones were produced in larger scale for evaluation in ELISA, kinetic studies and cell binding assays. The scFvs were expressed and purified by His-tag affinity purification. Purity of the scFvs was checked on SDS-PAGE Coomassie analysis and western blot to confirm the presence of His-tag. For titration ELISA, soluble scFvs were screened for their binding at fixed concentration of MUC1 target peptide 1 and control peptide 8 (see table 1) Results are found in FIGS. 8A and 8B. In addition, scFvs were screened for cross-reactivity on other MUC1 peptides 2, 3, 4 and 5 (see Table 1). Results are found in FIG. 9. Negative binding on unglycosylated MUC1 confirmed the library hypothesis that scFvs against the peptide backbone only cannot be selected. scFv D5 showed binding to monoTn-MUC1 glycopeptide for concentration >10 nM. Interestingly, clone D5 bind to peptide 3, while clones H3 and D3 also bind to peptide 3, but only at high concentrantion.

The clones that showed higher specificity for the target peptide 1 and zero cross-reactivity to IgA1 hinge region were chosen for further assessment. A3 and D2 clone demonstrated no binding to IgA1 hinge region while D3 and H3 demonstrated binding at high antibody concentrations. The rest of the scFvs recognized also IgA1 in addition to MUC1. Based on scFv specificity on titration ELISA A3, D2 and D3 scFvs were chosen for biological evaluation and kinetic studies.

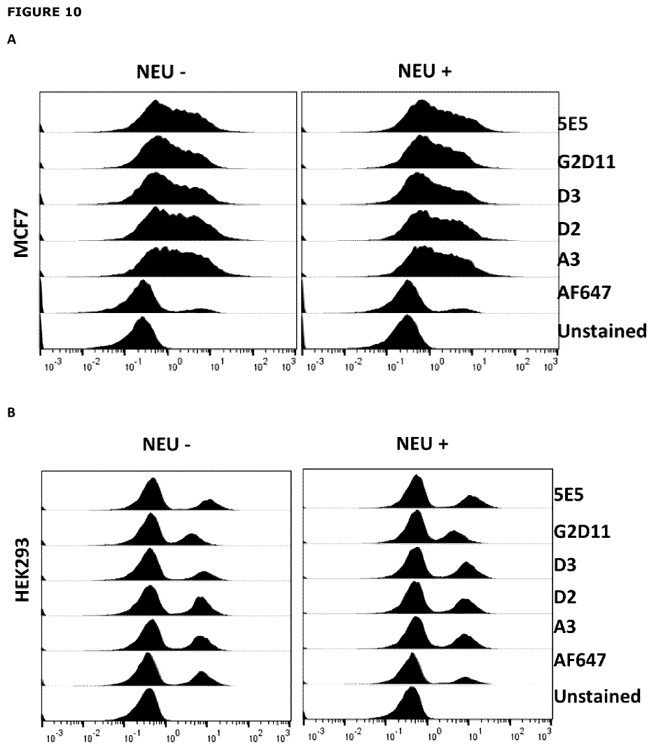

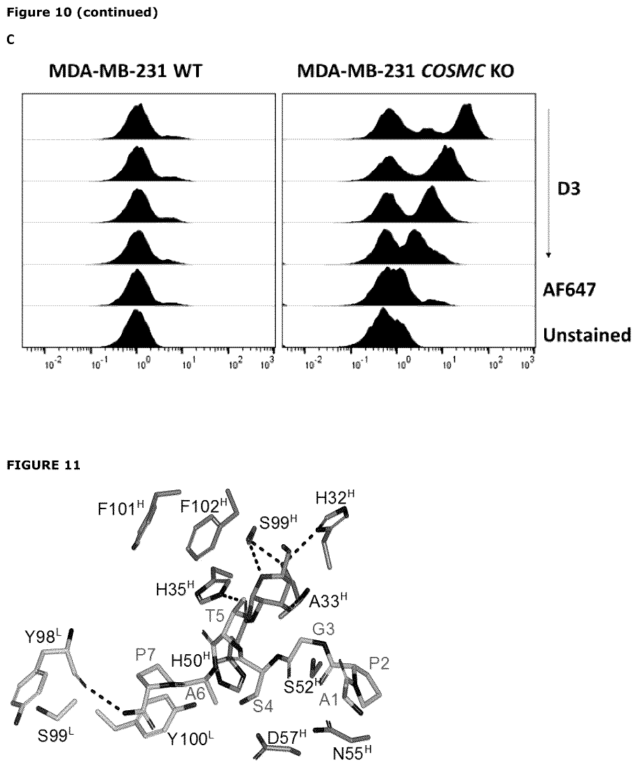

For cell binding assays, two breast adenocarcinoma cell lines were employed, MCF7 and MDA-MB-231 WT and COSMC KO cells. COSMC KO means a knock-out of the COSMC gene which is a chaperon required to help catalyze the transfer of Galb1-3 to the penultimale sugat Tn (GalNAc), generating the Tn-antigen and subsequent elongation. In cancer this COSMC is a frequent phenomenon and the results of exposure of Tn and STn-antigens on tumor proteins. mAb HMFG2 (anti-MUC1) was used as a positive control to confirm MUC1 expression on cells (data not shown) and 5E5 was used as control mAb to confirm the Tn glycoform presence on MUC1. In this assay, when binding occurs, the the main peak in the flow cytometry diagram shifts to the right compared to the controls. All three scFv A3, D2 and D3 showed positive binding on MDA-MD-231 COSMC KO cells and negative binding on WT cells (FIG. 8C). However, no positive binding was detected on MCF7 cells either for the selected MUC1 scFv clones or for 5E5 mAb control (FIG. 10A). Neuraminidase treatment did not enhance binding of 5E5 control and selected MUC1 scFv clonesD3, D2 and A4 on MCF7 cells (data not shown).

To exclude non-specific binding, scFv binding to HEK293 was tested since they do not express endogenously MUC1. No binding was detected neither before nor after neuraminidase treatment (FIG. 10B).

To further strengthen the concept of VL contribution in combotope specific recognition, the binding affinity (KD) of G2D11 and the newly identified MUC1 scFv clones were determined with BLI. To determine the on- and off-rates to calculate a dissociation constant, the biotinylated target peptide 1 was immobilized on streptavidin sensor tips. The only clones that showed higher binding affinity compared to G2D11 was clone D3 (Table 3).

TABLE 3
Affinity studies for A3, D2, D3 scFvs. Binding curves were fitted
in a 2:1 binding model (heterogeneous binding model). The obtained
KD1 and KD2 of the new clones in comparison to G2D11 validate
the contribution of the VL chain in peptide backbone binding.
Kinetic experiments were repeated two times.

| scFv | KD (M) | KD2 (M) |
|---|---|---|
| G2D11 | 5.6E−8 | 2.28E−7 |
| A3 | 3.5E−8 | 1.95E−5 |
| D2 | 2.3E−8 | 3.4E−8 |
| D3 | 2.6E−11 | 2.4E−7 |

Interestingly, the curves did not fit to a 1:1 binding model but instead in a 2:1 binding model (heterogeneous binding model). Therefore steady state kinetics cannot be calculated and in fact two KD values, KD and KD2, were obtained. One explanation for this is that VH-G2D11 binds bisTn in two orientations which makes it also more flexible in binding to Tn-structures in contrast to e.g. 5E5 that prefer a mono-Tn attached to a threonine (Thr) and less bis-Tn structures.

Based on the above, it is concluded that clones with peptide binding (combotopes) get an increase affinity (p-nM) compared to G2D11 (60 nM). More antigen-antibody bonds interact to improve specificity and increase affninity.

### 3.3 Sequence Analysis

Individual scFv sequence analysis revealed information about the CDR regions on the VL chains. scFvs D3, D5, H3 shared the MUC1 specific binding motif YSY in CDR3 like in 5E5 which is a requirement for peptide backbone binding interactions as it has been showed previously with crystallography (FIG. 11). More specifically, Y98^{L} and Y100^{L} are contributing to the peptide binding. scFvs A3 and D2 shared the motif WNY while scFvs B5 and A2 had the motif SSY (Table 4). In all scFvs as well as the 5E5 mAb the Y100^{L} is conserved. CDR1 and CDR2 has some variations that can be linked to the target peptide sequence and size (e.g W50^{L}) residue in cloe proximity to the peptide backbone.

TABLE 4
VL CDR3 sequences of the selected scFvs compared to 5E5. ScFvs
D5, D5 and H3 share the same YSY motif with 5E5, A3 and D2
have WNY as motif while B5 and A2 have the SSY motif.

| mAb | CDR3_VL* |
|---|---|
| 5E5 | _{QND}YSY_{PLTF} |
| D3 | _{QQY}YSY_{PLTF} |
| D5 | _{QQY}YSY_{PLTF} |
| H3 | _{QQY}YSY_{PLTF} |
| A3 | _{LQH}WNY_{PLTF} |
| D2 | _{LQH}WNY_{PLTF} |
| B5 | _{QQY}SSY_{PLTF} |
| A2 | _{QQY}SSY_{PLTF} |

*SEQ ID NOs 89-96 in sequence listing.

The obtained data correlate with current known information (x-ray and interacting residues, with specificities) from reference mAb 5E5. It demonstrates that the present library concept can enrich and select sequences with same features as obtained with immunized mice and hybridoma targeting the same antigen. This is a major step forward and with an animal free rapid system. The approach also provides a very large number of additional clones for evaluation with similar or different VL-sequence oprions that potentially could be better or different binders. The methods provides a relative (to hybridoma) controlled and systematic approach identifying large numbers of candidates for evaluation.

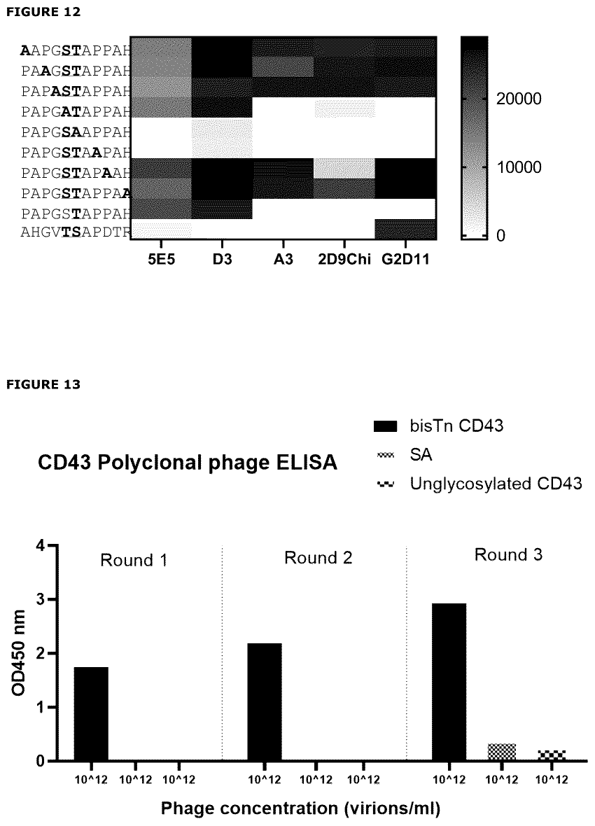

3.4 Binding Profiles of the Selected Novel scFvs Compared with Known Anti-Tn Antibodies Glycopeptides from Table 5 were printed on microarray chip, and the binding to these petides by scFv D3 and scFv A4 was compared with binding by scFv 5E5, scFv 2D9Chi, and scFv G2D11. 2D9Chi comprises the 2D9 VL domain and G2D11 VH domain. Results are presented in FIG. 12 (for simplicity, the heat map shows amino acids 9-19 of the peptides in Table 5).

TABLE 5
Glycopeptide sequences of 20-mer MUC1 peptides
on microarray chip. Alanine (Ala) substitutions
are shown in bold. GalNAc Glycosylation sites
are shown in bold and underlined.

| Peptide no. | Glycopeptide sequence | SEQ ID NO. |
|---|---|---|
| 20 | VTSAPDTRAAPGSTAPPAHG | 69 |
| 21 | VTSAPDTRPAAGSTAPPAHG | 70 |
| 22 | VTSAPDTRPAPASTAPPAHG | 71 |
| 23 | VTSAPDTRPAPGATAPPAHG | 72 |
| 24 | VTSAPDTRPAPGSAAPPAHG | 73 |
| 25 | VTSAPDTRPAPGSTAAPAHG | 74 |
| 26 | VTSAPDTRPAPGSTAPAAHG | 75 |
| 27 | VTSAPDTRPAPGSTAPPAAG | 76 |
| 28 | APGSTAPPAHGVTSAPDTRP | 77 |
| 29 | VTSAPDTRPAPGSTAPPAHG | 78 |

It was found that substitution of Thr with Ala as well as VTSA epitope abolished svFV 5E5 binding. Same epitope profile is demonstrated by scFv D3. It was shown that mono-Tn is not sufficient to get scFv G2D11 to bind, but scFv G2D11 binds to two adjacent Tn antigens. As previously disclosed herein, it is the VH domain of scFv G2D11 which is responsible for Tn binding. It was now further shown in this binding study, that if peptide binding is associated (such as in scFV D3—i.e. having VH domain of G2D11 and a VL domain found in the library screening), then the VH domain of G2D11 can support monoTn binding.

ScFv 2D9Chi binds to two adjacent Tn antigens only in Ser-Thr sequence, contrary to scFV G2D11 which also binds two adjacent Tn antigens in the Thr-Ser sequence. ScFv A3 showed the same epitope recognition as 2D9Chi. Exchange of Pro residue with Ala, abolishes the binding of all scFvs against the glycopeptide.

### Example 4: Concept Evaluation—CD43 as First Example

To assess the potentials of the Tn template library beyond MUC1, CD43 was chosen as the first target to identify binders following the same procedure.

Target peptide 9 (see table 1) was immobilized on streptavidin beads and three selection rounds were performed. To determine efficient phage selection, polyclonal phage ELISA and nanopore sequencing took place. Both polyclonal phage ELISA (FIG. 13) and sequencing confirmed phage and sequence enrichments between the rounds. However, in the case of CD43 the sequences were grouped based on the combinations of CDR1, CDR2 and CDR3 as it is not evident which CDR is responsible for peptide backbone binding as in the case of MUC1 (Table 6).

TABLE 6
Table lists the ten most enriched VL sequences as CDR1, CDR2, CDR3
combinations. Values (show the number of reads for every combination divided by
the total number of reads in every round

| | | | | | 1^{st} | 2^{nd} | 3^{rd} |
|---|---|---|---|---|---|---|---|
| Rank | CDR1 | CDR2 | CDR3 | Naive | round | round | round |
| 1 | KASQDVSTAVA | SASYRYT | QQYNSYPYT | 0.13 | 2.88 | 3.04 | 5.42 |
| 2 | KASQDVSTAVA | SASYRYS | QQYNSYPLT | 0.05 | 2.07 | 2.71 | 5.12 |
| 3 | KASQDVSTAVA | SASYRYT | QQHYSTPYT | 0.15 | 2.78 | 2.20 | 4.33 |
| 4 | KASQDVGTAVA | WASTRHT | QQYSSYPYT | 10.07 | 2.96 | 2.97 | 4.07 |
| 5 | KASQDVGTAVA | WASTRHT | QQYNSYPYT | 0.00 | 2.56 | 2.20 | 3.59 |
| 6 | KASQDVSTAVA | SASYRYS | QQYNSYPYT | 0.12 | 2.22 | 1.99 | 3.51 |
| 7 | KASQNVGTAVA | SASNRYT | QQYSSYPYT | 0.05 | 1.61 | 2.18 | 3.32 |
| 8 | KASQDVSTAVA | SASYRYT | QQYNSYPLT | 0.04 | 1.49 | 2.08 | 3.32 |
| 9 | KASQNVGTNVA | SASYRYS | QQYNSYPYT | 0.39 | 1.56 | 1.95 | 3.10 |
| 10 | KASQDVGTAVA | WASTRHT | QQYSSYPLT | 10.00 | 1.28 | 1.82 | 2.88 |

*SEQ ID NOs 97-126 in sequence listing.

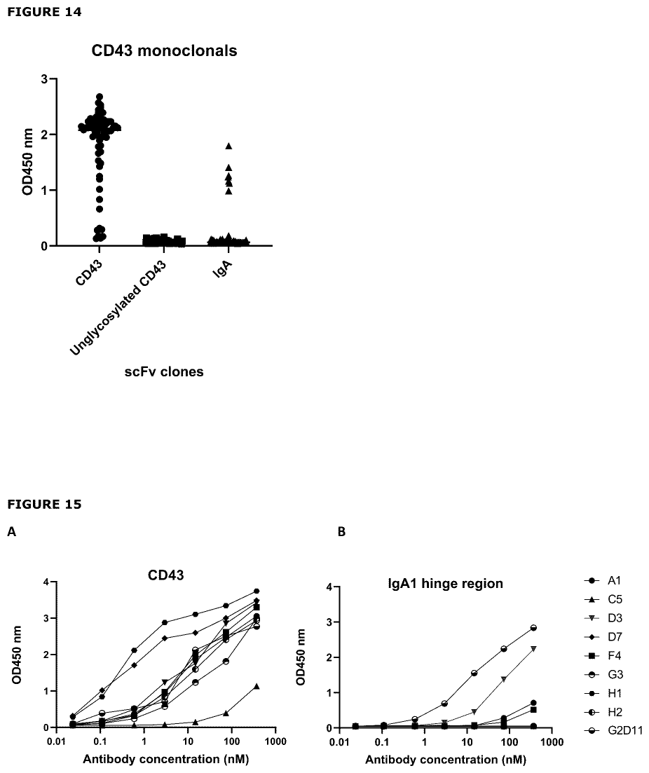

61 clones were picked, assessed for their binding on monoclonal scFv ELISA against target peptide 9 and control peptides 7 and 10 (see Table 1) (results in FIG. 14) and VL sequences were obtained. Based on the monoclonal ELISA results ten clones that showed high specificity for the target peptide and zero or low cross reactivity were selected for further evaluation.

The ten clones (named ori, H1, -A1, F4, C5, A7, D3, G3, D7, H2) were tested for their binding specificity on titration ELISA as a first step. These ten clones are represented by SEQ ID NOs. 12-21 in the sequences listing. Only their VL-domain is listed for the scFv. VH domain is same of the scFV of all clones—i.e. the VH of G2D11 (SEQ ID NO 1). The VL and VH are joined by the peptide linker (GGGS)₅.

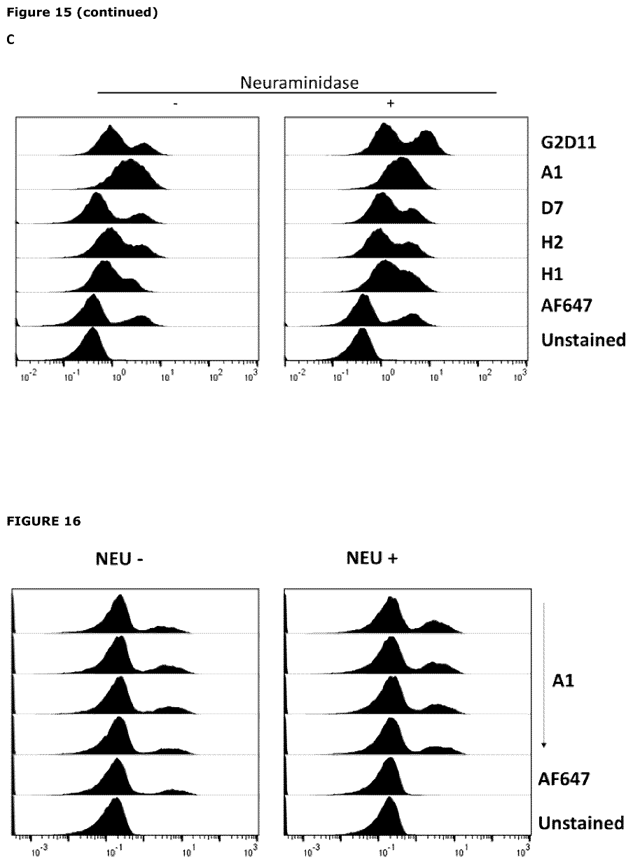

All scFvs showed high binding specificity for the CD43 target peptide (peptide no. 9), except C5 that showed the lowest binding specificity (FIG. 15A). A7 and D3 clone showed high binding affinity for IgA1 hinge region peptide, while A1 and F4 showed some cross-binding only at high concentration (FIG. 15B). The rest of the scFvs demonstrated no binding to IgA1 peptide. scFvs A1, D7, H1 and H2 were chosen for further assessment in cell binding assays and kinetic studies with BLI.

For biological evaluation, the leukemia Jurkat cells were chosen as they express high level of Tn antigen as a single nucleotide base deletion results in a frameshift and truncation of COSMC chaperone. Cells were treated with neuraminidase and stained in both cases. All of the four clones stained positively the Jurkat cells and the staining was enhanced upon neuraminidase treatment (FIG. 15C). scFvs were tested additionally on HEK293 cells as they do not express naturally CD43 (FIG. 16).

Finally, to confirm the VL contribution in the peptide binding, kinetic studies were performed with BLI. As in the case of MUC1 scFvs clones, the binding curves were fitted in a 2:1 binding model (heterogeneous ligand) and two KD were obtained (Table 7).

TABLE 7
Affinity studies with BLI to determine the VL contribution
in the peptide backbone binding. Curves were fitted
in 2:1 model (heterogeneous binding model).

| scFv | KD (M) | KD2 (M) |
|---|---|---|
| A1 | 8.9E−8 | 1.7E−7 |
| D7 | 1.9E−10 | 2.5E−7 |
| H2 | 2.4E−9 | 9.4E−8 |
| H1 | 2.4E−11 | 3.1E−8 |

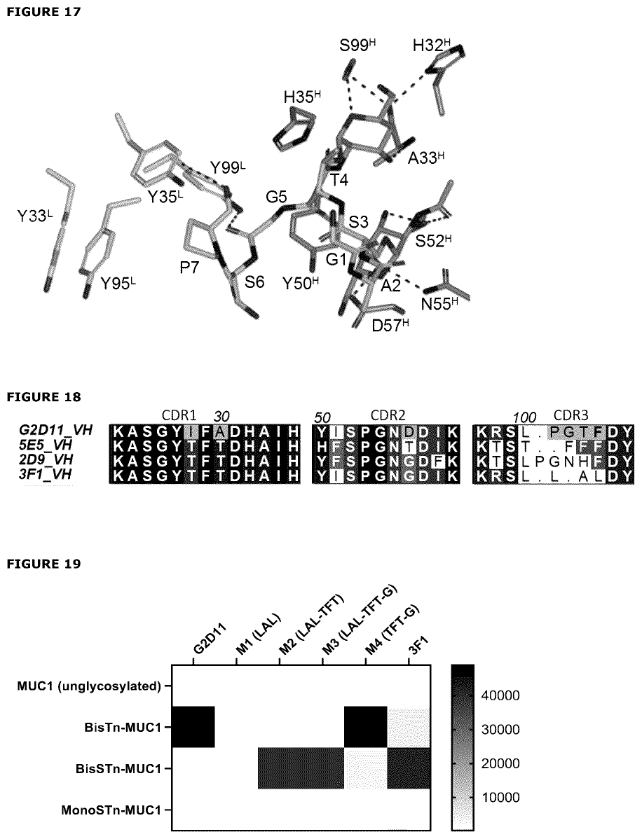

Sequence analysis of the selected nine scFv gave information about the CRD1 and CDR3 region of the VL chains, as demonstrated by X-ray. X-ray experiments of ori-CD43 scFv (SEQ ID NO. 12) with CD43-GAS*T*GSP peptide (where asterisk denotes a GalNac residue) (SEQ ID NO. 56), revealed Y99^{L} is required for specific peptide backbone binding interaction (FIG. 17). Interestingly, all scFvs had the Y99^{L} apart from D3, D7 and H1. D3 scFv possess a L99^{L} while D7 and H1 a W99^{L} (Table 8). D3 with the L99^{L} showed cross-binding to IgA hinge region peptide contrary to D7 and H1 that showed specific interaction with CD43 peptide and no cross-reactivity with TnIgA hinge or TnMUC1. However, scFv C5 that has a Y99^{L} also cross reacted with the control peptide.

TABLE 8
CDR3 sequences of the VL chains.
scFv A1, C5, F4 and G3 have an Tyr994,
D3 a Leu994, whereas D7 and H1 a Trp994.

| mAb | CDR3_VL* |
|---|---|
| A1 | _{QHHYGTP}Y_{TF} |
| C5 | _{QQHYSTP}Y_{TF} |
| D3 | _{QQYNSYP}L_{TF} |
| D7 | _{QQYYSYP}W_{TF} |
| F4 | _{QHFWSTP}Y_{TF} |
| G3 | _{QQYYSYP}Y_{TF} |
| H1 | _{QQYYSYP}W_{TF} |
| H2 | _{QQYYSYP}Y_{TF} |

*SEQ ID NOs 127-134 in sequence listing.

With this CD43 example, a second target example was provided with a different peptide sequence but still mucin-like (amino acid features for O-glycosylation, high content of S, T, P, etc.), and enrichment of VL-domain sequences with a unique CDR fingerprint especially for CDR1 and CDR3 was demonstrated, and also confirmed by x-ray. The additional CDR1 interactions further increase specificity as demonstrated with ELISA (no cross-reactivity to TnIgA or TnMUC1). This demonstrates that the antibody library concept of the present invention can target other Tn-peptide/proteins with different peptide sequence and obtain VL-domain sequences with corresponding signatures but different from above VL-domain sequences that targets Tn-MUC1.

### Example 5: STn-Template Library

5.1 Alignment of G2D11 with 3F1

Antibody 3F1 is a known anti STn antibody (Prendergast et al 2017). Sequence comparison between G2D11 VH domain (SEQ ID NO. 1) and 3F1 VH domain (SEQ ID NO. 25) (see FIG. 18) identified amino acid positions in CDR1 and CDR3 domains of G2D11 that are potentially responsible for STn glycan binding—specifically modifying the amino acid residues of G2D11 VH domain as follows: I28T, A30T, P101L, de/G102, T103A and F104L, seemed promising for changing the Tn glycan binding specificity of VH-G2D11 to STn glyan binding specificity.

5.2 G2D11 Mutant with STn Binding

G2D11 VH-domain mutants were prepared based on the above identified amino acid positions potentially relevant for STn specificity:
M1 (LAL): P101L, de/G102, T103A and F104L;
M2 (LAL-TFT): I28T, A30T, P101L, de/G102, T103A and F104L;
M3 (LAL-TFT-G): I28T, A30T, D56G, P101L, de/G102, T103A and F104L; and
M4 (TFT-G): I28T, A30T, and D56G.

Microaray data for binding to glycopeptides by scFV G2D11, 3F1, and mutants (M1-4). is illustrated in FIG. 19. The mutants M1-M4 are mutants of scFV G2D11 comprising the above mentioned selected mutations in the VH domain. It was found that for the LAL mutant M1, Tn binding was lost but no STn binding observed; while LAL+TFT mutant M2 generated STn binding and no Tn binding. These VH-domain mutations are sufficient to switch Tn to STn binding. Note that LAL is a requirement for the shift. No LAL mutation (TFT mutation only), Tn remains as a binder.

Hence, it was found that combination of mutation in VH chain, more precisely in CDR1 (IFA to TFT) and in CDR3 (PGTF to LAL), shifted the binding capacity from Tn to STn glycoform (FIG. 19).

This, combined with the previous VH sequence alignment (Example 1.2) shows that the following amino acid residues are required VH residues to have binding towards bisSTn O-glycans are T28, T30, H32, A33, H35, Y50, S52, N55, D57, S99, L101, A102 and L103, with reference to SEQ ID NO 28.

### 5.3 STn-Template Library

Chain shuffling the mutated VH chain (SEQ ID NO. 28) (i.e. the G2D11 VH domain comprising the IFA to TFT mutation in CDR1 and PGTF to LAL in CDR3) with a pool of naïve VL domains from naïve mice a second phage display library was generated, termed the STn-template library.

### Example 6: MUCe as Proof of Concept in STn Template Library

To investigate the potentials of the STn template library, MUC1 was used a proof of concept. Two peptides, target peptide 6 and 7 (see Table 1), were immobilized on NHS beads and three selection round were performed as described previously. Nanopore sequencing after each round took place and the VL diversity between the two targets peptides was compared.

Table 9 and Table 10 provides the top ten most enriched combinations and their percentages in every round for bisSTn-MUC1 and monoSTn-MUC1, respectively. In both selections, specific MUC1 binding motif Tyr-X-Tyr can be identified. The VL-domain sequences confirm that the STn-library can be used, and generates similar data/clones as obtained for TnMUC1.

TABLE 9
Sequence enrichment for bis STn MUC1 biopanning selection. The top ten
most enriched combinations and their percentages in every round.

| Rank | CDR3* | Naive | 1^{st} round | 2^{nd} round | 3^{rd} round |
|---|---|---|---|---|---|
| 1 | QQYNSYPYT | 1.45 | 1.27 | 21.68 | 48.10 |
| 2 | QQYYSYPRT | 0.07 | 0.39 | 6.76 | 15.62 |
| 3 | QQHYSTPRT | 0.76 | 1.41 | 3.57 | 7.62 |
| 4 | SQSTHVPRT | 2.77 | 5.18 | 22.94 | 7.59 |
| 5 | QQYYSYPWT | 0.12 | 0.49 | 1.76 | 5.10 |
| 6 | QQHYSTPWT | 2.41 | 2.07 | 1.12 | 4.44 |
| 7 | HQYHRSPPT | 4.22 | 3.00 | 6.71 | 4.38 |
| 8 | HQYHRSPRT | 0.19 | 0.50 | 4.01 | 2.58 |
| 9 | QQRSSYPYT | 0.54 | 0.61 | 3.59 | 0.85 |
| 10 | QQHYSTPYT | 1.04 | 0.23 | 0.69 | 0.66 |

*SEQ ID NOS 135-144 in sequence listing.

TABLE 10
Sequence enrichment for mono STn MUC1 biopanning selection. The top
ten most enriched combinations and their percentages in every round.

| Rank | CDR3* | Naive | 1^{st} round | 2^{nd} round | 3^{rd} round |
|---|---|---|---|---|---|
| 1 | QQHYSTPRT | 0.76 | 1.18 | 13.55 | 32.96 |
| 2 | QQYNSYPYT | 1.45 | 1.56 | 7.38 | 16.72 |
| 3 | QQYYSYPRT | 0.07 | 0.54 | 7.20 | 14.13 |
| 4 | KQYHRSPPT | 0.00 | 0.14 | 2.82 | 10.45 |
| 5 | HQYHRSPPT | 4.22 | 8.48 | 11.31 | 8.27 |
| 6 | QQYYSYPPT | 0.05 | 0.21 | 7.67 | 7.21 |
| 7 | QQWSSNPYT | 1.28 | 2.85 | 9.32 | 3.04 |
| 8 | QQHYSTPWT | 2.41 | 5.35 | 6.89 | 2.73 |
| 9 | SQSTHVPRT | 2.77 | 2.33 | 9.00 | 1.74 |
| 10 | HQYHRSPRT | 0.19 | 0.42 | 2.64 | 0.56 |

*SEQ ID NOs 145-154 in sequence listing.

The enriched VL-domain sequences contains the same features as seen for Tn-MUC1 and further consolidate the fingerprint related to MUC1 peptide target.

Based on monoclonal scFv ELISA specificity and VL sequence comparison, three clones were selected for purification. These three clones (named C4, D3, C7) are represented by SEQ ID NOs. 22-24 in the sequences listing. Only their VL-domain is listed for the scFv. VH domain is same for the scFV of all clones—i.e. the mutated VH chain of G2D11 (SEQ ID NO. 28) (i.e. the G2D11 VH domain comprising the IFA to TFT mutation in CDR1 and PGTF to LAL in CDR3, as disclosed in example 5). The VL and VH are joined by the peptide linker (GGGS)₅.

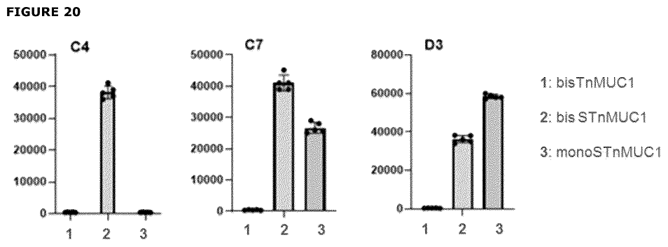

Microarray analysis (FIG. 20) showed that both bis-STn-MUC1 and mono-STn-MUC1 binders, but not Tn-binders were obtained.

### Example 7: Kinetic Affinities for MUC1 and CD43 Specific scFvs

Table 11 summarizes the kinetic affinities for the MUC1 and CD43 specific scFvs identified using the antibody library according to the present invention.

TABLE 11

| Target | scFv | KD (M) | KD2 (M) | ka (1/Ms) | ka2 | kdis (1/s) | kdis2 | KD (%) | KD2 (%) |
|---|---|---|---|---|---|---|---|---|---|
| MUC1 | G2D11 | 5.609E−08 | 2.287E−07 | 1.426E04 | 1.592E05 | 7.999E−04 | 3.641E−02 | 81 | 19 |
| | A3 | 3.542E−08 | 1.952E−05 | 1.878E04 | 5.062E02 | 6.651E−04 | 9.882E−03 | 31 | 69 |
| | D2 | 2.305E−08 | 3.435E−08 | 7.843E03 | 1.150E04 | 1.808E−04 | 3.950E−04 | 60 | 40 |
| | D3 | 1.974E−11 | 6.781E−07 | 2.461E04 | 6.391E04 | 4.858E−07 | 4.334E−02 | 66 | 34 |
| CD43 | A1 | 8.959E−08 | 1.713E−07 | 2.878E06 | 2.584E03 | 2.579E−01 | 4.426E−04 | 2 | 98 |
| | D7 | 1.893E−10 | 2.574E−07 | 1.377E04 | 7.220E03 | 2.607E−06 | 1.859E−03 | 97 | 3 |
| | H2 | 2.454E−09 | 9.415E−08 | 1.153E04 | 1.248E06 | 2.830E−05 | 1.175E−01 | 88 | 12 |
| | H1 | 2.465E−11 | 3.176E−06 | 1.819E04 | 1.622E03 | 4.484E−07 | 5.152E−03 | 34 | 66 |

### Example 8: Mono Tn/STn scFv Binders

Enrichment of mono Tn MUC1 binders in the bisTn MUC1 biopanning as it was shown in the polyclonal phage ELISA (FIG. 5, example 2) indicated that the library can also be panned with peptides with single GalNac attached to them.

Indeed, MUC1 target peptides 2 and 3 (see table 1) were used to perform three rounds of selection. Polyclonal phage ELISA did not show phage enrichment, however sequencing of picked clones shared specific MUC1 sequences.

This is an important observation and may—without wishing to be bound by theory—be explained as follows: First, we know that monoTn-peptides are not binding to G2D11 (at least not without VL-contribution, see our ref Persson et al 2017). Mutations of VH (as stated above in example 1) also confirms this. G2D11 VH has high affinity (60 nM) for bisTn contraty monoTn. This also leads to that panning with monoTn provides much less enrichment of clones as many of them are bisTn-binders. However, if quantity decreases, the quality increases and whatever binders that remain are most likely combotope binders with VL-peptide contribution to increase binding affinities. This is an advantage and demonstrates that the stringency can be influenced by manipulating the VH-domain binding strength either by removing one Tn or provide Tn-inhibitor or mutate the VH so it becomes less specific for bisTn, other panning conditions etc. Thus, we could see that selected scFv clones evaluated has high ratio of combotope binders and very little of bisTn-hapten binders. The nanopore sequence enrichment phage data also confirms this with monoSTn-MUC1. In contrast, enrichment with bisTn/STn-binders has much higher hapten binders and less combotope binders. All together, the antibody library concept of the present invention can be used to target monoTn/STn-peptide binders and not only bisTn/Stn-peptide binders, and this significantly increases utility as Tn/STn are situated as orfan, bis or in larger clusters.

### Example 9: Humanizing Mouse mAbs

A humanized scFv is generated by humanising the VL and VH immunoglobulin domains derived from the murine-originated antiCD43. Humanisation of VL and VH is performed in scFv format as follows:

### Protocol:

1. Identify the complementarity-determining regions (CDRs): The CDRs are the parts of the antibody that interact with the antigen. The CDRs in the mouse monoclonal antibody are identified. Amino acid sequences of murine antiCD43 scFv VH and VL originated via phage display are numbered according to IGMT, and the IMGT-defined CDR residues are identified. Amino acids involved in binding but not included in the IMGT-defined CDR residues are also included as CDRs.

2. Design a humanized version of the antibody: Computational modeling tools are used to design a humanized version of the mouse monoclonal antibody. The goal is to maintain the antigen-binding specificity of the mouse antibody while replacing the mouse-derived CDRs with human-derived CDRs. From the amino acid sequence encompassing the 3 complementarity determining regions (CDRs) within the VL and VH domains, VH and VL sequences are generated in which the CDRs are masked. These sequences are used as input for the Basic Local Alignment Search Tool (BLAST) algorithm (Altschul et al., 1997) to identify similar frameworks from human V gene (heavy, kappa, lambda) germline databases. In addition, framework 4 amino acid sequence from the murine antiCD43 scFv VH and VL are used to identify similar human J gene segments.

Human V and J gene segments are chosen as template frameworks based on their identity to antiCD43 sequence, in-house analysis of individual and pairing frequency of V genes and previous experience of the use of particular templates for legacy humanisation.

The chosen human V gene frameworks are compared to the respective murine VH and VL sequences to identify potential sites that could undergo back-mutation to the corresponding mouse amino acid at that position. In-house collated evidences rules for the importance of certain framework positions in the likely maintenance of CDR conformation (and antigen binding affinity) are used to identify back-mutations considered most significant (primary mutations) and those of lower significance (secondary mutations). The extent of spatial clustering of the identified back-mutations is examined by analysing the crystallized molecular structure of mouse antiCD3. Initial humanised VH and VL sequences are generated by constructing a straight graft of the mouse CDRs into the chosen human germline templates. The apparent spatial clustering of back-mutation sites is used to reduce the potential number of variants of back-mutation containing humanised chains by introducing spatially-clustered mutations simultaneously.

Immunogenicity of the final humanised sequences is evaluated using online tools in AbYsis.

3. Clone the humanized antibody: The genes for the heavy and light chains of the humanized antibody are cloned into expression vectors. These vectors are used to produce the humanized antibody in a suitable expression system, such as mammalian cells. Amino acid sequences of humanised VH and VL chains are combined in the VL-VH orientation. scFv sequences comprised a (G4S)4 linker between VL and VH chains, and a C-terminal exa-His tag. scFv protein sequences are reverse translated and codon optimised.

All codon optimised DNA sequences are modified to include 5′ and 3′ adaptors suitable for HiFi cloning in the pET22b (+) and synthesised. DNA sequences are synthesised by TwistBioscience as double-stranded fragments (gBlocks). pET22b (+) backbone is linearized by PCR and the product is treated with DnpI and cleaned with Monarch DNA&PCR cleanup. The gBlocks is inserted in pET22b (+) using the NEBuilder® HiFi DNA Assembly Cloning Kit. Ligation mixtures are transformed into DH5a competent E. coli and positive transformants selected on plates of LB agar supplemented with 100 ug/ml carbenicillin. Colonies for putative clones are cultured, plasmid DNA extracted, and DNA subjected to Sanger sequencing to identify correct clones.

Transient transfection of scFv-encoding construct into HEK 2936E suspension culture 250 μg of DNA for each plasmid construct is transfected using 293 Fectin transfection reagent into separate 250 ml cultures of HEK 293 6E cells (at a viable cell density of 1.85×10® cell/ml. The cultures are placed into a shaking 37° C. incubator at 124 rpm with 5% CO2. At 48 and 72 hours the cultures are supplemented with 6.2 ml tryptone (200 g/l) and 6.2 ml 3M fructose respectively.

From 48 hours post-transfection the viability (%) and viable cell density (cell/ml) of each culture are measured every 24 hours using a Vi-Cell cell counter and viability analyser (Beckman Coulter). Once the cultures have reached <70% viability, the cultures are harvested via centrifugation at 4415×g for thirty minutes at 4° C. and filtered via 0.22 μm Millipore filter. The filtered supernatants are stored at 4° C. until required for protein purification.

4. Purify the humanized antibody: The humanized antibody is purified using Single-Step Affinity Protein Purification. The scFv proteins are purified from the resulting supernatants via AKTA Express system (AKTA). The supernatant is loaded at 5 ml/minute onto a 5 ml HisTrap Excel column pre-equilibrated with Buffer A (50 mM HEPES pH 7.5, 400 mM NaCl, 20 mM Imidazole). Once loaded, the column is washed in two column volumes of Buffer A at 5 ml/minute back to baseline. The proteins are eluted in a step elution of 50% Buffer B (50 mM HEPES pH 7.5, 00 mM NaCl, 1M Imidazole). The column is held in three column volumes of 50% Buffer B. During this step elution 0.5 ml fractions are collected in the purification of 88A, then 1 ml fractions are collected in all subsequent purifications. The elution step is continued until returned to baseline followed by a washout step at three column volumes of 100% Buffer B.

A single peak is expected at 280 nM on the resulting chromatogram indicating the elution of the protein of interest. The fractions corresponding to this peak are pooled and transferred to a 5000 MW cut-off centrifugal concentrator. The sample is buffer exchanged from Buffer B into 60 ml PBS to separate the purified protein from the imidazole present in Buffer B. The samples are concentrated down to <1 ml. The concentration is determined via nanodrop and the purified protein is diluted in PBS to obtain a final concentration of 1 mg/ml. The final protein product is aliquoted and stored at −80° C. for future use.

5 pg aliquots of the purified samples in the batch are run under reducing conditions via SDS PAGE, displaying the purity and correct molecular weight of the purified proteins

5. Characterize the humanized antibody: The binding specificity and affinity of the humanized antibody to the target antigen is confirmed. Test for potential immunogenicity and other properties of the antibody such as stability and solubility.
Analytical Size Exclusion Chromatography (aSEC) for homogeneity of purified proteins
Mass Spectrometry or Peptide Mass Fingerprinting to confirm protein identity.
Biacore Analysis to confirm binding.

### REFERENCES

Blixt et al 2010. A High-throughput O-Glycopeptide Discovery Platform for Seromic Profiling. J Proteome Res. 2010 Oct. 1; 9(10): 5250-5261. doi:10.1021/pr1005229.
Blixt et al 2012. Analysis of Tn antigenicity with a panel of new IgM and IgG1 monoclonal antibodies raised against leukemic cells. Glycobiology. 2012 April; 22(4):529-42. doi: 10.1093/glycob/cwr178. Epub 2011 Dec. 5.
Clavero-Álvarez et al 2018. Humanization of Antibodies using a Statistical Inference Approach. Sci Rep. 2018 Oct. 4; 8(1):14820. doi: 10.1038/s41598-018-32986-y.
Karst et al. 2021. High-accuracy long-read amplicon sequences using unique molecular identifiers with Nanopore or PacBio sequencing. Nat Methods 18, 165-169 (2021).
Kjeldsen et al 1988. Preparation and characterization of monoclonal antibodies directed to the tumor-associated O-linked sialosyl-2-6 alpha-N-acetylgalactosaminyl (sialosyl-Tn) epitope. Cancer Res. 1988 Apr. 15; 48(8):2214-20.
Kudelka et al 2015. Simple Sugars to Complex Disease-Mucin-Type O-Glycans in Cancer. Adv Cancer Res. 2015; 126: 53-135. Doi: 10.1016/bs.acr.2014.11.002.
Li et al 2009. Resolving conflicting data on expression of the Tn antigen and implications for clinical trials with cancer vaccines. Mol Cancer Ther. 2009 April; 8(4):971-9. doi: 10.1158/1535-7163.MCT-08-0934. PMID: 19372570; PMCID: PMC2752371.
Macias-Leon et al 2020. Structural characterization of an unprecedented lectin-like antitumoral anti-MUC1 antibody. Chem Commun (Camb). 2020 Dec. 8; 56(96):15137-15140. doi: 10.1039/d0cc06349e.
Mazal et al 2013. Monoclonal antibodies toward different Tn-amino acid backbones display distinct recognition patterns on human cancer cells. Implications for effective immuno-targeting of cancer. Cancer Immunol Immunother. 2013 June; 62(6):1107-22. doi: 10.1007/s00262-013-1425-7. Epub 2013 Apr. 21. PMID: 23604173.
Oppezzo P et al 2000. Production and functional characterization of two mouse/human chimeric antibodies with specificity for the tumor-associated Tn-antigen. Hybridoma. 2000 June; 19(3):229-39. doi: 10.1089/02724570050109620. PMID: 10952411.
Persson et al. 2016. A combinatory antibody-antigen microarray assay for high-content screening of single-chain fragment variable clones from recombinant libraries. PLoS One 11, (2016).
Person et al. 2017. Epitope mapping of a new anti-Tn antibody detecting gastric cancer cells. Glycobiology, 2017, vol. 27, no. 7, 635-645. doi: 10.1093/glycob/cwxO33.
Prendergast et al 2017. Novel anti-Sialyl-Tn monoclonal antibodies and antibody-drug conjugates demonstrate tumor specificity and anti-tumor activity. MAbs. 2017 May/June; 9(4):615-627. doi: 10.1080/19420862.2017.1290752. Epub 2017 Feb. 22.
Sørensen et al 2006. Chemoenzymatically synthesized multimeric Tn/STn MUC1 glycopeptides elicit cancer-specific anti-MUC1 antibody responses and override tolerance. Glycobiology. 2006 February; 16(2):96-107. doi: 10.1093/glycob/cwjO44. Epub 2005 Oct. 5.
Tarp et al 2007. Identification of a novel cancer-specific immunodominant glycopeptide epitope in the MUC1 tandem repeat. Glycobiology vol. 17 no. 2 pp. 197-209, 2007. doi:10.1093/glycob/cwl061.
Yuasa et al 2012. Construction and expression of anti-Tn-antigen-specific single-chain antibody genes from hybridoma producing MLS128 monoclonal antibody. J Biochem. 2012 April; 151(4):371-81. doi: 10.1093/jb/mvs007. Epub 2012 Feb. 8. PMID: 22318767.

1. An antibody library for in-vitro identification of a specific antibody which binds a tumor cell, wherein each antibody in said library comprises
(i) a first antibody domain which binds a Tn- and/or STn-carbohydrate epitope of a glycoprotein of said tumor cell, and
(ii) a second antibody domain selected from a repertoire of second antibody domains, wherein the repertoire of second antibody domains comprises one or more second antibody domains which binds a peptide epitope of said glycoprotein of said tumor cell,
wherein said specific antibody is specific for a combination of said carbohydrate epitope and said peptide epitope of said glycoprotein.

2. The antibody library according to claim 1, wherein the carbohydrate epitope is covalently linked to the peptide epitope.

3. The antibody library according to claim 1, wherein the first antibody domain is a VH-domain, and wherein the second antibody domain is a VL-domain.

4. The antibody library according to claim 1, wherein the first antibody domain is a VH-domain, wherein the second antibody domain is a VL-domain, and wherein the antibodies in the library are scFv wherein the VH-domain is linked to the VL-domain via a peptide linker.

5. The antibody library according to claim 1, wherein the carbohydrate epitope is selected from mono-Tn, bis-Tn, mono-STn, bis-STn, and a combination of mono-Tn and mono-STn.

6. The antibody library according to claim 1, wherein the first antibody domain is a mono- or bis-Tn-binding VH domain, and wherein said VH-domain comprises an amino acid sequence having at least 90% sequence homology to SEQ ID NO. 1 and comprising amino acid residues H32, A33, H35, Y50, and S99 and/or amino acid residues S52, N55, and D57, with respect to SEQ ID NO. 1.

7. The antibody library according to claim 1, wherein the first antibody domain is a mono- or bis-STn-binding VH domain, and wherein said VH-domain comprises an amino acid sequence having at least 90% sequence homology to SEQ ID NO. 28 and comprising amino acid residues (i), T28, T30, H32, A33, H35, Y50, S99, L101, A102 and L103, (ii), T28, T30, S52, N55, D57, L101, A102 and L103, or (iii) T28, T30, H32, A33, H35, Y50, S52, N55, D57, S99, L101, A102 and L103, with respect to SEQ ID NO. 28.

8. The antibody library according to claim 1, wherein the first antibody domain does not contribute to or interfere with binding any peptide epitope.

9. The antibody library according to claim 1, wherein the repertoire of second antibody domains is generated from a naïve immune repertoire of VL-domains, an immunized immune repertoire of VL-domains, or a synthetically produced repertoire of VL-domains; preferably a naïve immune repertoire of VL-domains from an animal, such as a mouse or human.

10. The antibody library according to claim 1, wherein the antibody library is a phage display library.

11. A method for identifying an antibody for targeting a glycoprotein, comprising the steps of
i) preparing an antibody library according to claim 1, and
ii) screening said library to identify one or more tumor targeting antibodies.

12. The method according to claim 11, comprising biopanning of the antibody library using a glycopeptide or glycoprotein of said tumor cell, preferably an O-glycosylated peptide or protein, such as Tn-mucin or other O-glycosylated protein having mucin-like motif, wherein said glycopeptide or glycoprotein is used in purified form or expressed on a cell surface or tissue.

13. The method according to claim 11, wherein the antibody library is a phage display library prepared by a method comprising the steps 1) mRNA isolation from a spleen, 2) cDNA synthesis from said mRNA, 3a) amplification from said cDNA using a specific set of primers to obtain a first nucleic acid sequence encoding the VH domain, 3b) application from said cDNA using a mix of primers to obtain multiple nucleic acid sequences encoding the repertoire of VL domains, 4) assembly of the first nucleic acid sequence encoding the VH-domain and a second nucleic acid sequence from the multiple nucleic acid sequences encoding the VL-domain repertoire, to form a joint construct, 5) insertion of the construct into a phagemid vector, 6) insertion of the phagemid vector comprising the construct into E. coli to produce a bacterial library, 7) using the bacterial library for infection of phages to produce the phage display library.

14. A method for identifying a glycopeptide target, said target comprising a Tn and/or STn epitope and a peptide epitope, such as a glycopeptide target of a cancer cell, said method comprising the steps of
i) preparing an antibody library according to claim 1,
ii) incubating the antibody library with a sample comprising the glycopeptide target, and
iii) analyzing one or more antibody-peptide complexes obtained from step ii) to identify the amino acid sequence of the peptide epitope of said glycopeptide target.

15. A specific tumor cell binding antibody, comprising
(i) a VH-domain which binds a Tn- and/or STn-carbohydrate epitope of a glycoprotein of the tumor cell, and
(ii) a VL-domain which binds a peptide epitope of said glycoprotein of said tumor cell,
wherein the antibody is specific for a combination of said carbohydrate epitope and said peptide epitope of said glycoprotein,
with the provision that the antibody is not 5E5, 5F7 or 2D9.

16. The antibody according to claim 15, wherein the VH domain comprises
(i) a first amino acid sequence having at least 90% sequence homology to SEQ ID NO. 1, wherein the first amino acid sequence comprises amino acid residues H32, A33, H35, Y50, and S99 and/or amino acid residues S52, N55, and D57, with respect to SEQ ID NO. 1; or
(ii) a second amino acid sequence having at least 90% sequence homology to SEQ ID NO. 28, and wherein the second amino acid sequence comprises amino acid residues (i), T28, T30, H32, A33, H35, Y50, S99, L101, A102 and L103, (ii), T28, T30, S52, N55, D57, L101, A102 and L103, or (iii) T28, T30, H32, A33, H35, Y50, S52, N55, D57, S99, L101, A102 and L103, with respect to SEQ ID NO. 28.

17. The antibody according to claim 15, wherein the VL domain comprises an amino acid sequence selected from SEQ ID NO. 9-24.

18. A method of treating a cancer in a subject comprising administering a formulation comprising at least one antibody according to claim 15 to a patient in need thereof.

19. A method of diagnosing a cancerous condition in a subject comprising administering a formulation comprising at least one antibody according to claim 15 to the subject, and detecting the presence of an antigen-antibody complex comprising said at least one antibody according to claim 15 and a Tn- and/or STn-antigen.
